SEQUENCE LISTING (29 sequences)

SEQ ID NO: 1 - DNA, 3985 bp, Homo sapiens
ggctgagctgctgccgccgccgggcggacgggcggacgcgcggagctgggggcggcgcggcggggccggcggggcgcggcggggctgaccggccccgatgaggcggaaggagaagcggctcctgcaggcggtggcgctggtgctggcggccctggtcctcctgcccaacgtggggctttgggcgctgtaccgcgagcggcagcccgacggcacccctgggggatcgggggcggcggtggcgccggcggcgggacagggctcacacagtcgacaaaagaaaacgtttttcttgggagatgggcagaagctgaaggactggcatgacaaggaggccatccggagggacgctcagcgcgtaggaaatggagaacaaggaagaccttaccccatgaccgatgctgagagagtggatcaggcataccgagaaaatggatttaacatctacgtcagtgataaaatctccttgaatcgctctctcccagatatccggcacccaaactgcaacagcaagcgctacctggagacacttcccaacacaagcatcatcatccccttccacaacgagggctggtcctccctcctccgcaccgtccacagtgtgctcaatcgctcgcctccagagctggtcgccgagattgtactggtcgacgacttcagtgatcgagagcacctgaagaagcctcttgaagactacatggcccttttccccagtgtgaggattcttcgaaccaagaaacgggaagggctgataaggacccgaatgctgggggcctcagtggcaactggggatgtcatcacattcttggattcacactgtgaagccaatgtcaactggcttccccccttgcttgaccgcattgctcggaaccgcaagaccattgtgtgcccgatgattgatgtaattgaccatgacgactttcggtacgagacacaggcaggggatgccatgcggggagcctttgactgggagatgtactacaagcggatcccgatccctccagaactgcagaaagctgaccccagcgacccatttgagtctcccgtgatggccggtggactgttcgccgtggatcggaagtggttctgggaactcggcgggtatgacccaggcttggagatctggggaggggagcagtatgaaatctccttcaaggtgtggatgtgtgggggccgcatggaggacatcccctgctccagggtgggccatatctacaggaagtatgtgccctacaaggtcccggccggagtcagcctggcccggaaccttaagcgggtggccgaagtgtggatggatgagtacgcagagtacatttaccagcgccggcctgaataccgccacctctccgctggggatgtcgcagtccagaaaaagctccgcagctcccttaactgcaagagtttcaagtggtttatgacgaagatagcctgggacctgcccaaattctacccacccgtggagcccccggctgcagcttggggggagatccgaaatgtgggcacagggctgtgtgcagacacaaagcacggggccttgggctccccactaaggctagagggctgcgtccgaggccgtggggaggctgcctggaacaacatgcaggtattcaccttcacctggagagaggacatccggcctggagacccccagcacaccaagaagttctgctttgatgccatttcccacaccagccctgtcacgctgtacgactgccacagcatgaagggcaaccagctgtggaaataccgcaaagacaagaccctgtaccaccctgtcagtggcagctgcatggactgcagtgaaagtgaccataggatcttcatgaacacctgcaacccatcctctctcacccagcagtggctgtttgaacacaccaactcaacagtcttggaaaaattcaataggaactgagccctcatgtccccttggcaggccccccagggtctggcactcactgcagacttcctctttcaagggaggcagggcccctgtgggcactaggtgtaaaaggtgctggccaaatggttcagggtgaagagggctcttgattcaggggctggggtctgcctggtccttgagcccctgagttgtgggggtagggtgaagagcatatcccacaagaggccccacagggagcagagactgctttaatccctgctgacatcacggaaaagcaacagagccttttcaactttgtcactatgtccccttgaacattatgtgggagaacaccaaggtagcctaggccacccaaaagtgagtcctgcgaggttgcccagccctcagatggctctcctacatgatggtgctttagaaacaaaggtaaaatttgcctgtttggggcagcttttagtatcgatgccactcatctgcagcagaagagaaagaagtcctcttggggctttttagtttctgccgtcctggggggaacattgcagttactgcacagcttctgttctctgtcacaaccccaggtgatttggtccggtcaaaggccatacttggggccctaagagtgttcagtattgaatgctgatcagctgccaggtgaggagtcagaagagggagcccccctagacatttctttgcagctatggacatgcgggatatctccccctgctctctgggtatttgaaatgtcaattttagcactctccaggcacaaggacagcccagcaccagctttacagggcagtgtttcagatggccctgagcccacggaaaaggccaggtagacctccaaactagaaatgctggctgatttgccctgatccatgcttccatttccctgtctctcttccccaggcaattactggcctcaaaagaggaacagaggtgctgcgaggtgctcacctcacagagtctggaggcctccaggatcaactgtgggcaaagtgcctgcctctgacctcatcatggttctagttctcatacagaactccagaatttttaaagaactctataattggattgcaaactaggatgctacataggattctggtattccacatccaatatggatttctagaatgctgtgattaaaggagccagccaggtgtaatacagtcaaggcagcccccagcctagagacaatctgtgaaatccaaagttggtggtgttgggaaagcagggggacatgtgtccctcagctcagcagaggctgtggtacaacatggtccttggtgaagacctgcacccctggaacctcccaccatcatcacaactgtagtctcatttgcagtggagaaaagaacccgacgtcccacagccagatatacacccagctccatgccagcccttcatgtttaccttttgctttgttaattacatgtcagactcctagagggcctccagactaataggaagcatttctgtaaccaacctgccacccactgattcagaaatggaaatcacattccacaatctatggcttccaccagctagcccaggaaatacttgaaatcagcattccaattagtgttgagtctcttgattgtgtcatttaccaattaaataactgagacctaagtctgggaacagagccacgaatctgcctttgagatgctggcagatctcaaggccatcaattattgggggagggagggacaaacactcccaatcatccaccagtcagactgaatgtgtagctggcgaggaattacttccacttctggcccagcacaagccctgctttggccacctgtctgcaagagaggcggcccctgtgcttgcaacgcttacgtgttgatcccagtgtccttttccaaatgagtgctgtagctttagaagtggccctctatagaaagaagtcaaaagatgaggccccttctagaatctaggataacaagagtgttgacagtttgaggagtcgaattgagattcatcatcaaagagcaatgcagcgtcgttaaaataaaaactgtgccttttaaaaagaaaaatgcaaatatagagcaaatccctaaacttgaaaaaaaaaaaaaaaaa

SEQ ID NO: 2 - protein, 603 aa, Homo sapiens
MRRKEKRLLQAVALVLAALVLLPNVGLWALYRERQPDGTPGGSGAAVAPAAGQGSHSRQKKTFFLGDGQKLKDWHDKEAIRRDAQRVGNGEQGRPYPMTDAERVDQAYRENGFNIYVSDKISLNRSLPDIRHPNCNSKRYLETLPNTSIIIPFHNEGWSSLLRTVHSVLNRSPPELVAEIVLVDDFSDREHLKKPLEDYMALFPSVRILRTKKREGLIRTRMLGASVATGDVITFLDSHCEANVNWLPPLLDRIARNRKTIVCPMIDVIDHDDFRYETQAGDAMRGAFDWEMYYKRIPIPPELQKADPSDPFESPVMAGGLFAVDRKWFWELGGYDPGLEIWGGEQYEISFKVWMCGGRMEDIPCSRVGHIYRKYVPYKVPAGVSLARNLKRVAEVWMDEYAEYIYQRRPEYRHLSAGDVAVQKKLRSSLNCKSFKWFMTKIAWDLPKFYPPVEPPAAAWGEIRNVGTGLCADTKHGALGSPLRLEGCVRGRGEAAWNNMQVFTFTWREDIRPGDPQHTKKFCFDAISHTSPVTLYDCHSMKGNQLWKYRKDKTLYHPVSGSCMDCSESDHRIFMNTCNPSSLTQQWLFEHTNSTVLEKFNRN

SEQ ID NO: 3 - DNA, 1809 bp, Homo sapiens
atgaggcggaaggagaagcggctcctgcaggcggtggcgctggtgctggcggccctggtcctcctgcccaacgtggggctttgggcgctgtaccgcgagcggcagcccgacggcacccctgggggatcgggggcggcggtggcgccggcggcgggacagggctcacacagtcgacaaaagaaaacgtttttcttgggagatgggcagaagctgaaggactggcatgacaaggaggccatccggagggacgctcagcgcgtaggaaatggagaacaaggaagaccttaccccatgaccgatgctgagagagtggatcaggcataccgagaaaatggatttaacatctacgtcagtgataaaatctccttgaatcgctctctcccagatatccggcacccaaactgcaacagcaagcgctacctggagacacttcccaacacaagcatcatcatccccttccacaacgagggctggtcctccctcctccgcaccgtccacagtgtgctcaatcgctcgcctccagagctggtcgccgagattgtactggtcgacgacttcagtgatcgagagcacctgaagaagcctcttgaagactacatggcccttttccccagtgtgaggattcttcgaaccaagaaacgggaagggctgataaggacccgaatgctgggggcctcagtggcaactggggatgtcatcacattcttggattcacactgtgaagccaatgtcaactggcttccccccttgcttgaccgcattgctcggaaccgcaagaccattgtgtgcccgatgattgatgtaattgaccatgacgactttcggtacgagacacaggcaggggatgccatgcggggagcctttgactgggagatgtactacaagcggatcccgatccctccagaactgcagaaagctgaccccagcgacccatttgagtctcccgtgatggccggtggactgttcgccgtggatcggaagtggttctgggaactcggcgggtatgacccaggcttggagatctggggaggggagcagtatgaaatctccttcaaggtgtggatgtgtgggggccgcatggaggacatcccctgctccagggtgggccatatctacaggaagtatgtgccctacaaggtcccggccggagtcagcctggcccggaaccttaagcgggtggccgaagtgtggatggatgagtacgcagagtacatttaccagcgccggcctgaataccgccacctctccgctggggatgtcgcagtccagaaaaagctccgcagctcccttaactgcaagagtttcaagtggtttatgacgaagatagcctgggacctgcccaaattctacccacccgtggagcccccggctgcagcttggggggagatccgaaatgtgggcacagggctgtgtgcagacacaaagcacggggccttgggctccccactaaggctagagggctgcgtccgaggccgtggggaggctgcctggaacaacatgcaggtattcaccttcacctggagagaggacatccggcctggagacccccagcacaccaagaagttctgctttgatgccatttcccacaccagccctgtcacgctgtacgactgccacagcatgaagggcaaccagctgtggaaataccgcaaagacaagaccctgtaccaccctgtcagtggcagctgcatggactgcagtgaaagtgaccataggatcttcatgaacacctgcaacccatcctctctcacccagcagtggctgtttgaacacaccaactcaacagtcttggaaaaattcaataggaac

SEQ ID NO: 4-10 - skipped (7 entries)

SEQ ID NO: 11 - DNA, 4158 bp, Homo sapiens
tcggcggaagatggcggccggcgaaaggagctggtgcctgtgcaagttgttgaggtttttttattcattattcttccctgggctcattgtatgtggaactttatgtgtgtgtttggtcattgtcctttggggaatcagactgctgctacagagaaagaaaaaattagtgtcaactagcaaaaatgggaaaaatcaaatggtgattgcattttttcatccatactgcaatgctggtggaggaggagaaagagttttatggtgtgctttaagagccctgcagaaaaagtatcctgaagcagtttatgttgtttataccggcgatgttaatgtcaacggtcaacagatactagaaggtgctttcagaagatttaacatcagattaattcacccagtgcagtttgtttttttaaggaaacgctatcttgtggaagattcactgtatcctcacttcacactgctgggccaaagtctaggatccatttttcttggctgggaagctctaatgcagtgtgttcctgatgtttacattgattcaatgggatacgcttttacgcttcctctgtttaagtatatagggggttgccaagttggaagctatgttcattatcctactatcagcaccgacatgctctctgtagtgaagaatcaaaatattggatttaataatgcagccttcattaccaggaatccttttctcagcaaagtaaagctcatctactactatttatttgcttttatttatggacttgttggttcttgcagtgatgtagtcatggtcaattcttcttggacactaaaccatattctctcactatggaaagttgggaattgcactaacattgtttatccaccttgtgatgtgcagacatttctggacattcccttacatgagaaaaagatgaccccaggacatttgctggtttctgttggccagtttaggccggaaaagaatcatccattgcagatcagagcctttgctaaattgctgaataagaagatggttgagtcacctccttcgcttaaacttgtcctcattggaggttgtcgtaacaaagatgatgaacttagggtaaaccaactgagaaggctgtctgaggatttaggagttcaagaatatgtggaatttaaaataaacattccatttgatgaattaaagaattatttgtctgaagcaacaattggtctgcataccatgtggaacgagcattttgggattggagttgtggagtgtatggcagctggcacaattatccttgcacacaattcggggggcccaaagcttgacattgtggttcctcacgaaggagatataactggctttctggctgagagtgaagaagactatgctgaaactatcgctcacattctttccatgtctgcagaaaagagactccaaatcagaaaaagtgctcgtgcatctgtaagcagattctctgatcaggaatttgaagtgacattcctatcatctgtggaaaagttatttaagtaatgccatatctgtaaaattaaagatattttatataaactggttaaacaccttcatatgtaaatatttttctaaattcaattcggcggaagatggcggccggcgaaaggagctggtgcctgtgcaagttgttgaggtttttttattcattattcttccctgggctcattgtatgtggaactttatgtgtgtgtttggtcattgtcctttggggaatcagactgctgctacagagaaagaaaaaattagtgtcaactagcaaaaatgggaaaaatcaaatggtgattgcattttttcatccatactgcaatgctggtggaggaggagaaagagttttatggtgtgctttaagagccctgcagaaaaagtatcctgaagcagtttatgttgtttataccggcgatgttaatgtcaacggtcaacagatactagaaggtgctttcagaagatttaacatcagattaattcacccagtgcagtttgtttttttaaggaaacgctatcttgtggaagattcactgtatcctcacttcacactgctgggccaaagtctaggatccatttttcttggctgggaagctctaatgcagtgtgttcctgatgtttacattgattcaatgggatacgcttttacgcttcctctgtttaagtatatagggggttgccaagttggaagctatgttcattatcctactatcagcaccgacatgctctctgtagtgaagaatcaaaatattggatttaataatgcagccttcattaccaggaatccttttctcagcaaagtaaagctcatctactactatttatttgcttttatttatggacttgttggttcttgcagtgatgtagtcatggtcaattcttcttggacactaaaccatattctctcactatggaaagttgggaattgcactaacattgtttatccaccttgtgatgtgcagacatttctggacattcccttacatgagaaaaagatgaccccaggacatttgctggtttctgttggccagtttaggccggaaaagaatcatccattgcagatcagagcctttgctaaattgctgaataagaagatggttgagtcacctccttcgcttaaacttgtcctcattggaggttgtcgtaacaaagatgatgaacttagggtaaaccaactgagaaggctgtctgaggatttaggagttcaagaatatgtggaatttaaaataaacattccatttgatgaattaaagaattatttgtctgaagcaacaattggtctgcataccatgtggaacgagcattttgggattggagttgtggagtgtatggcagctggcacaattatccttgcacacaattcggggggcccaaagcttgacattgtggttcctcacgaaggagatataactggctttctggctgagagtgaagaagactatgctgaaactatcgctcacattctttccatgtctgcagaaaagagactccaaatcagaaaaagtgctcgtgcatctgtaagcagattctctgatcaggaatttgaagtgacattcctatcatctgtggaaaagttatttaagtaatgccatatctgtaaaattaaagatattttatataaactggttaaacaccttcatatgtaaatatttttctaaattcaatctcatttgtcaaatcattttactttagaaaacagacaaaatttccttttagaataaaaggaagtgttgaaaagaaaatggatgactagccttcggcttccattcttggtatacatgagagaggctggctgctgagatgaatgtgaaccaggttgcagagaatctggctttgagccaccaggaagaactagtggatttgccaaaaaactaccccttgagtgaaaatgaagatgagggggacagtgatggagagagaaagcatcaaaagcttctggaagcaatcatttcccttgatggaaagaataggcggaaattggctgagaggtctgaggctagtctgaaagtgtcagagttcagtgtcagttctgaaggatcaggagaaaagctgggccttgcagatctgcttgagcccgttaaaacttcatcttctttggccactgtaaaaaagcaactgaatagagtcaaatcaaagaaggtggtggagttacctcttaacaaagaaaaaattgaacagatccacagagaagtagcattcagtaaaacctcacaggtcctctccaaatgggaccctatcatcctgaagaaccagcaggcagagcagctggtttttcccctggggaaggagcagccagccattgctcccattgaacatgcgctcagtggctggaaggcaagaactcccctggagcaggaaatttttaacctcctccataagaacaagcagccagtgacagatcctttactgactcccatggaaaaggcctctctccaagccatgagcctggaagaggcaaagatgcaccgagcagagcttcagagggctcgggctctgcagtcctactatgaggccaaggctcgaaaagagaagaaaatcaaaagtaaaaagtatcacaaagtcgtgaagaaaggaaaggccaagaaagccttaaaagagtttgagcagctacagaaggttaatccaactgtggcactggaaaaaaaaaaaaaaaaaaaaaaaaaaaaaaaaaaaaaaaaaaaaaaaaa

SEQ ID NO: 12 - protein, 492 aa, Homo sapiens
MAAGERSWCLCKLLRFFYSLFFPGLIVCGTLCVCLVIVLWGIRLLLQRKKKLVSTSKNGKNQMVIAFFHPYCNAGGGGERVLWCALRALQKKYPEAVYVVYTGDVNVNGQQILEGAFRRFNIRLIHPVQFVFLRKRYLVEDSLYPHFTLLGQSLGSIFLGWEALMQCVPDVYIDSMGYAFTLPLFKYIGGCQVGSYVHYPTISTDMLSVVKNQNIGFNNAAFITRNPFLSKVKLIYYYLFAFIYGLVGSCSDVVMVNSSWTLNHILSLWKVGNCTNIVYPPCDVQTFLDIPLHEKKMTPGHLLVSVGQFRPEKNHPLQIRAFAKLLNKKMVESPPSLKLVLIGGCRNKDDELRVNQLRRLSEDLGVQEYVEFKINIPFDELKNYLSEATIGLHTMWNEHFGIGVVECMAAGTIILAHNSGGPKLDIVVPHEGDITGFLAESEEDYAETIAHILSMSAEKRLQIRKSARASVSRFSDQEFEVTFLSSVEKLFK

SEQ ID NO: 13 - DNA, 3044 bp, Homo sapiens
atggcggccggcgaaaggagctggtgcctgtgcaagttgttgaggtttttttattcattattcttccctgggctcattgtatgtggaactttatgtgtgtgtttggtcattgtcctttggggaatcagactgctgctacagagaaagaaaaaattagtgtcaactagcaaaaatgggaaaaatcaaatggtgattgcattttttcatccatactgcaatgctggtggaggaggagaaagagttttatggtgtgctttaagagccctgcagaaaaagtatcctgaagcagtttatgttgtttataccggcgatgttaatgtcaacggtcaacagatactagaaggtgctttcagaagatttaacatcagattaattcacccagtgcagtttgtttttttaaggaaacgctatcttgtggaagattcactgtatcctcacttcacactgctgggccaaagtctaggatccatttttcttggctgggaagctctaatgcagtgtgttcctgatgtttacattgattcaatgggatacgcttttacgcttcctctgtttaagtatatagggggttgccaagttggaagctatgttcattatcctactatcagcaccgacatgctctctgtagtgaagaatcaaaatattggatttaataatgcagccttcattaccaggaatccttttctcagcaaagtaaagctcatctactactatttatttgcttttatttatggacttgttggttcttgcagtgatgtagtcatggtcaattcttcttggacactaaaccatattctctcactatggaaagttgggaattgcactaacattgtttatccaccttgtgatgtgcagacatttctggacattcccttacatgagaaaaagatgaccccaggacatttgctggtttctgttggccagtttaggccggaaaagaatcatccattgcagatcagagcctttgctaaattgctgaataagaagatggttgagtcacctccttcgcttaaacttgtcctcattggaggttgtcgtaacaaagatgatgaacttagggtaaaccaactgagaaggctgtctgaggatttaggagttcaagaatatgtggaatttaaaataaacattccatttgatgaattaaagaattatttgtctgaagcaacaattggtctgcataccatgtggaacgagcattttgggattggagttgtggagtgtatggcagctggcacaattatccttgcacacaattcggggggcccaaagcttgacattgtggttcctcacgaaggagatataactggctttctggctgagagtgaagaagactatgctgaaactatcgctcacattctttccatgtctgcagaaaagagactccaaatcagaaaaagtgctcgtgcatctgtaagcagattctctgatcaggaatttgaagtgacattcctatcatctgtggaaaagttatttaagtaatgccatatctgtaaaattaaagatattttatataaactggttaaacaccttcatatgtaaatatttttctaaattcaattcggcggaagatggcggccggcgaaaggagctggtgcctgtgcaagttgttgaggtttttttattcattattcttccctgggctcattgtatgtggaactttatgtgtgtgtttggtcattgtcctttggggaatcagactgctgctacagagaaagaaaaaattagtgtcaactagcaaaaatgggaaaaatcaaatggtgattgcattttttcatccatactgcaatgctggtggaggaggagaaagagttttatggtgtgctttaagagccctgcagaaaaagtatcctgaagcagtttatgttgtttataccggcgatgttaatgtcaacggtcaacagatactagaaggtgctttcagaagatttaacatcagattaattcacccagtgcagtttgtttttttaaggaaacgctatcttgtggaagattcactgtatcctcacttcacactgctgggccaaagtctaggatccatttttcttggctgggaagctctaatgcagtgtgttcctgatgtttacattgattcaatgggatacgcttttacgcttcctctgtttaagtatatagggggttgccaagttggaagctatgttcattatcctactatcagcaccgacatgctctctgtagtgaagaatcaaaatattggatttaataatgcagccttcattaccaggaatccttttctcagcaaagtaaagctcatctactactatttatttgcttttatttatggacttgttggttcttgcagtgatgtagtcatggtcaattcttcttggacactaaaccatattctctcactatggaaagttgggaattgcactaacattgtttatccaccttgtgatgtgcagacatttctggacattcccttacatgagaaaaagatgaccccaggacatttgctggtttctgttggccagtttaggccggaaaagaatcatccattgcagatcagagcctttgctaaattgctgaataagaagatggttgagtcacctccttcgcttaaacttgtcctcattggaggttgtcgtaacaaagatgatgaacttagggtaaaccaactgagaaggctgtctgaggatttaggagttcaagaatatgtggaatttaaaataaacattccatttgatgaattaaagaattatttgtctgaagcaacaattggtctgcataccatgtggaacgagcattttgggattggagttgtggagtgtatggcagctggcacaattatccttgcacacaattcggggggcccaaagcttgacattgtggttcctcacgaaggagatataactggctttctggctgagagtgaagaagactatgctgaaactatcgctcacattctttccatgtctgcagaaaagagactccaaatcagaaaaagtgctcgtgcatctgtaagcagattctctgatcaggaatttgaagtgacattcctatcatctgtggaaaagttatttaag

SEQ ID NO: 14-19 - skipped (6 entries)

SEQ ID NO: 20 - protein, 559 aa, Homo sapiens [sequence omitted]

SEQ ID NO: 21 - protein, 559 aa, Rattus sp.
MRKFAYCKVVLATSLVWVLLDMFLLLYFSECNKCEEKKERGLPAGDVLELVQKPHEGPGEMGKPVVIPKEDQEKMKEMFKINQFNLMASEMIAFNRSLPDVRLEGCKTKVYPDSLPTTSVVIVFHNEAWSTLLRTVHSVINRSPRHMIEEIVLVDDASERDFLKRPLESYVKKLKVPVHVIRMEQRSGLIRARLKGAAVSKGQVITFLDAHCECTVGWLEPLLARIKHDRRTVVCPIIDVISDDTFEYMAGSDMTYGGFNWKLNFRWYPVPQREMDRRKGDRTLPVRTPTMAGGLFSIDRDYFQEIGTYDAGMDIWGGENLEISFRIWQCGGTLEIVTCSHVGHVFRKATPYTFPGGTGQIINKNNRRLAEVWMDEFKNFFYIISPGVTKVDYGDISSRVGLRHKLQCKPFSWYLENIYPDSQIPRHYFSLGEIRNVETNQCLDNMARKENEKVGIFNCHGMGGNQVFSYTANKEIRTDDLCLDVSKLNGPVTMLKCHHLKGNQLWEYDPVKLTLQHVNSNQCLDKATEEDSQVPSIRDCTGSRSQQWLLRNVTLPEIF

SEQ ID NO: 22 - protein, 559 aa, Mus sp.
MRKFAYCKVVLATSLVWVLLDMFLLLYFSECNKCEEKQERGLPAGDVLELVQKPHEGPGEMGKPVVIPKEDQEKMKEMFKINQFNLMASEMIALNRSLPDVRLEGCKTKVYPDNLPTTSVVIVFHNEAWSTLLRTVHSVINRSPRHMIEEIVLVDDASERDFLKRPLESYVKKLKVPVHVIRMEQRSGLIRARLKGAAVSRGQVITFLDAHCECTAGWLEPLLARIKHDRRTVVCPIIDVISDDTFEYMAGSDMTYGGFNWKLNFRWYPVPQREMDRRKGDRTLPVRTPTMAGGLFSIDRDYFQEIGTYDAGMDIWGGENLEISFRIWQCGGTLEIVTCSHVGHVFRKATPYTFPGGTGQIINKNNRRLAEVWMDEFKNFFYIISPGVTKVDYGDISSRLGLRRKLQCKPFSWYLENIYPDSQIPRHYFSLGEIRNVETNQCLDNMARKENEKVGIFNCHGMGGNQVFSYTANKEIRTDDLCLDVSKLNGPVTMLKCHHLKGNQLWEYDPVKLTLQHVNSNQCLDKATEEDSQVPSIRDCTGSRSQQWLLRNVTLPEIF

SEQ ID NO: 23 - protein, 559 aa, Bos sp.
MRKFAYCKVVLATSLIWVLLDMFLLLYFSECNKCDEKKERGLPAGDVLEPVQKPHEGPGEMGKPVVIPKEDQEKMKEMFKINQFNLMASEMIALNRSLPDVRLEGCKTKVYPDNLPTTSVVIVFHNEAWSTLLRTVHSVINRSPRHMLEEIVLVDDASERDFLKRPLESYVKKLKVPVHVIRMEQRSGLIRARLKGAAVSKGQVITFLDAHCECTVGWLEPLLARIKHDRKTVVCPIIDVISDDTFEYMAGSDMTYGGFNWKLNFRWYPVPQREMDRRKGDRTLPVRTPTMAGGLFSIDRDYFQEIGTYDAGMDIWGGENLEISFRIWQCGGTLEIVTCSHVGHVFRKATPYTFPGGTGQIINKNNRRLAEVWMDEFKNFFYIISPGVTKVDYGDISSRLGLRHKLQCRPFSWYLENIYPDSQIPRHYFSLGEIRNVETNQCLDNMARKENEKVGIFNCHGMGGNQVFSYTANKEIRTDDLCLDVSKLNGPVTMLKCHHLKGNQLWEYDPVKLTLQHVNSNQCLDKATDEDSQVPSIRDCSGSRSQQWLLRNVTLPEIF

SEQ ID NO: 24 - protein, 559 aa, Sus sp.
MRKFAYCKVVLATSLIWVLLDMFLLLYFSECNKCDEKKERGLPAGDVLEPVQKPHEGPGEMGKPVVIPKEDQDKMKEMFKINQFNLMASEMIALNRSLPDVRLEGCKTKVYPDNLPTTSVVIVFHNEAWSTLLRTVHSVINRSPRHMLEEIVLVDDASERDFLKRPLESYVKKLKVPVHVIRMEQRSGLIRARLKGAAVSKGQVITFLDAHCECTVGWLEPLLARIKHDRKTVVCPIIDVISDDTFEYMAGSDMTYGGFNWKLNFRWYPVPQREMDRRKGDRTLPVRTPTMAGGLFSIDRDYFQEIGTYDAGMDIWGGENLEISFRIWQCGGTLEIVTCSHVGHVFRKATPYTFPGGTGQIINKNNRRLAEVWMDEFKTFFYIISPGVTKVDYGDISSRLGLRHKLQCRPFSWYLENIYPDSQIPRHYSSLGEIRNVETNQCLDNMARKENEKVGIFNCHGMGGNQVFSYTANKEIRTDDLCLDVSKLNGPVTMLKCHHLKGNQLWEYDPVKLTLQHVNSNQCLDKATEEDSQVPSIRDCSGSRSQQWLLRNVTLPEIF

SEQ ID NO: 25 - protein, 612 aa, Caenorhabditis elegans
MLSVGGGRSAVCRAVIATSIVWLLIDVVILFYYLDPSTSQQQPFPEDNRILNRARRIEPLPPAAQHDSDPDAHPIQPEKQEKQVYPVDKETANQLRKLMETQAFGPGYHGQGGTGVTVPEDKKTIKEKRFLENQFNVVASEMISVNRTLPDYRSDACRTSGNNLKTAGMPKTSIIIVFHNEAWTTLLRTLHSVINRSPRHLLEEIILVDDKSDRDYLVKPLDSYIKMFPIPIHLVHLENRSGLIRARLTGSEMAKGKILLFLDAHVEVTDGWLEPLVSRVAEDRKRVVAPIIDVISDDTFEYVTASETTWGGFNWHLNFRWYAVPKRELNRRGSDRSMPIQTPTIAGGLFAIDKQFFYDIGSYDEGMQVWGGENLEISFRVWMCGGSLEIHPCSRVGHVFRKQTPYTFPGGTAKVIHHNAARTAEVWMDEYKAFFYKMVPAARNVEAGDVSERKKLRETLQCKSFKWYLENIYPEAPLPADFRSLGAIVNRFTEKCVDTNGKKDGQAPGIQACHGAGGNQAWSLTGKGEIRSDDLCLSSGHVYQIGSELKLERCSVSKINVKHVFVFDDQAGTLLHKKTGKCVTGADQRVTLDECGLGRKDQMWQLEGYQSP

SEQ ID NO: 26 - protein, 589 aa, Caenorhabditis elegans
MLPRMLKMKTVGTVLAVIWLFGLAFIYVQSTSSSLRPPGRHPPPLPQLDPLIPQNPPQNDEIRPKKSAPPIPTINLAEDTTIHERTEKDVTWKTFDVEKFLNKGKWHQGEDKYKANSFNQEASDALNPTRKIPDSREPQCRDVDYSKVGMQPTTVIITYHNEARSSLLRTVFSVFNQSPEELLLEIVLVDDNSQDVEIGKELAQIQRITVLRNNQREGLIRSRVKGAQVARAPVLTFLDSHIECNQKWLEPLLARIAENPKAVVAPIIDVINVDNFNYVGASADLRGGFDWTLVFRWEFMNEQLRKERHAHPTAPIRSPTMAGGLFAISKEWFNELGTYDLDMEVWGGENLEMSFRVWQCGGSLEIMPCSRVGHVFRKKHPYTFPGGSGNVFQKNTRRAAEVWMDEYKAIYLKNVPSARFVNFGDITDRLAIRDRLQCKSFKWYLENVYPQLEIPRKTPGKSFQMKIGNLCLDSMARKESEAPGLFGCHGTGGNQEWVFDQLTKTFKNAISQLCLDFSSNTENKTVTMVKCENLRPDTMVVEKNGWLTQGGKCLTVNQGSGGDWLIYGAHCELNNGAQRWIFEKLDTYE

SEQ ID NO: 27 - protein, 626 aa, Caenorhabditis elegans
MIIFKKKAILKVLLLVPVFWICSLIFFAATSNDSSQIGSNNDLANKIAEANFHPKAAKQDVIQGFGPPIEPEPVVENNKVEEEEQPGGNLAKPKFMVDPNDPIYKKGDAAQAGELGKAVVVDKTKLSTEEKAKYDKGMLNNAFNQYASDMISVHRTLPTNIDAECKTEKYNENLPRTSVIICFHNEAWSVLLRTVHSVLERTPDHLLEEVVLVDDFSDMDHTKRPLEEYMSQFGGKVKILRMEKREGLIRARLRGAAVATGEVLTYLDSHCECMEGWMEPLLDRIKRDPTTVVCPVIDVIDDNTFEYHHSKAYFTSVGGFDWGLQFNWHSIPERDRKNRTRPIDPVRSPTMAGGLFSIDKEYFEKLGTYDPGFDIWGGENLELSFKIWMCGGTLEIVPCSHVGHVFRKRSPYKWRTGVNVLKRNSIRLAEVWLDDYKTYYYERINNQLGDFGDISSRKKLREDLGCKSFKWYLDNIYPELFVPGESVAKGEVRNSAVQPARCLDCMVGRHEKNRPVGTYQCHGQGGNQYWMLSKDGEIRRDESCVDYAGSDVMVFPCHGMKGNQEWRYNHDTGRLQHAVSQKCLGMTKDGAKLEMVACQYDDPYQHWKFKEYNEAKAIEHGAKPPS

SEQ ID NO: 28 - protein, 618 aa, Caenorhabditis elegans
MIASLIRSRRRSRRCVVYSVFLFGFLALWGSFALALVFLSDMYIGEDQISTQKAIKPIARSNYHVVVGHYNGNLPEDKKRNLTSEELNANLYAPHDDWGEGGAGVSHLTPEQQKLADSTFAVNQFNLLVSDGISVRRSLPEIRKPSCRNMTYPDNLPTTSVIIVYHNEAYSTLLRTVWSVIDRSPKELLKEIILVDDFSDREFLRYPTLDTTLKPLPTDIKIIRSKERVGLIRARMMGAQEAQGDVLTFLDSHCECTKGWLEPLLTRIKLNRKAVPCPVIDIINDNTFQYQKGIEMFRGGFNWNLQFRWYGMPTAMAKQHLLDPTGPIESPTMAGGLFSINRNYFEELGEYDPGMDIWGGENLEMSFRIWQCGGRVEILPCSHVGHVFRKSSPHDFPGKSSGKVLNTNLLRVAEVWMDDWKHYFYKIAPQAHRMRSSIDVSERVELRKKLNCKSFKWYLQNVFQDHFLPTPLDRFGRMTSSSNSSVCLAWTLRSSGIKTASTADCLKIFHKTQLWLYTGDRRIRTDEHLCLSVVQLLHTTSDWKIQLKECAGFDTEYWDFKPKIGRFQNRKTGLCLASPDIFDPTKDEFNPPIVQKCRSSNDRQNWTITEMSWLPEHP

SEQ ID NO: 29 - protein, 579 aa, Caenorhabditis elegans
MLRYIIPRKKGTFVIAAFLTVAFFCIVAYHRNDRRRTKFQFPDIEKYAEELVRLPETWNGELHQIPNYTAPREGPGEKGKPVVLTGKDAELGQADMKKWFMNVHASDKISLDRDVPDPRIQACKDIKYDYAALPKTSVIIIFTDEAWTPLLRTVHSVINRSPPELLQEVILLDDNSKRQELQEPLDEHIKRFGGKVRLIRKHDRHGLIRAKLAGAREAVGDIIVFLDSHCEANHGWLEPIVQRISDERTAIVCPMIDSISDNTLAYHGDWSLSTGGFSWALHFTWEGLSEEEQKRRTKPTDYIRSPTMAGGLLAANREYFFEVGGYDEEMDIWGGENLEISFRAWMCGGSIEFIPCSHVGHIFRAGHPYNMTGRNNNKDVHGTNSKRLAEVWMDDYKRLYYMHREDLRTKDVGDLTARHELRKRLNCKPFKWFLDNIAKGKFIMDEDVVAYGALHTVVSGTRMCTDTLQRDEKMSQLLGVFHCQGKGSSPQLMSLSKEGNLRRENTCASEENGNIRMKTCSKKAQFNERWAYENKMIRNLKSGKCMSTANLKPGDNAIVVECDEKDEHQKWNFIDPAKA

(19) United States
(12) Patent Application Publication
(10) Pub. No.: US 2005/0203049 A1
(43) Pub. Date: Sep. 15, 2005

(54) 47169 and 33935, novel human glycosyl transferases and uses therefor

(75) Inventors: Rachel E. Meyers, Newton, MA (US); Mark Williamson, Saugus, MA (US)

Correspondence Address: MILLENNIUM PHARMACEUTICALS, INC., 40 Landsdowne Street, Cambridge, MA, 02139, US

(73) Assignee: Millennium Pharmaceuticals, Inc.

(21) Appl. No.: 11/120,749

(22) Filed: May 3, 2005

Related U.S. Application Data
(63) Continuation of application No. 10/001,851, filed on Nov. 20, 2001.
(60) Provisional application No. 60/249,939, filed on Nov. 20, 2000.

(51) Int. Cl. A61K 48/00; C12N 15/85

(52) U.S. Cl. 514/44; 435/455

(57) ABSTRACT

The invention provides isolated nucleic acids molecules, designated 47169 and 33935 nucleic acid molecules, which encode novel glycosyl transferases. The invention also provides antisense nucleic acid molecules, recombinant expression vectors containing 47169 and 33935 nucleic acid molecules, host cells into which the expression vectors have been introduced, and non-human transgenic animals in which a 47169 or 33935 gene has been introduced or disrupted. The invention still further provides isolated 47169 and 33935 proteins, fusion proteins, antigenic peptides and anti-47169 and anti-33935 antibodies. Diagnostic methods utilizing compositions of the invention are also provided.

### CROSS-REFERENCE TO RELATED APPLICATIONS

This application is entitled to priority pursuant to 35 U.S.C. § 119(e) to U.S. provisional patent application 60/249,939 which was filed on Nov. 20, 2000.

### STATEMENT REGARDING FEDERALLY SPONSORED RESEARCH OR DEVELOPMENT

Not Applicable.

### REFERENCE TO A MICROFICHE APPENDIX

Not Applicable.

### BACKGROUND OF THE INVENTION

Many proteins, both within a cell and particularly on cell surfaces, can be post-translationally modified by addition of one or more carbohydrate residues to an amino acid residue thereof. Lipid moieties can also be modified by addition of carbohydrate moieties thereto.

Cell surface carbohydrate moieties have a significant role in binding between the cell to which they are attached and cells, viruses, and proteins that bind with that cell. Furthermore, glycosylation of secreted proteins can affect the binding specificity and capacity of the secreted protein to interact with other entities. In addition, glycosylation of proteins involved in transcription of genes or translation of the transcripts produced by such transcription can affect the degree to which a gene is expressed (i.e., the amount of an encoded protein produced by a cell).

Protein glycosylation can add saccharide or oligosaccharide (i.e., carbohydrates comprising more than one sugar entity) moieties to hydroxyl moieties (e.g., serine, threonine, and hydroxylysine-residue side chains), to amino moieties (e.g., asparagine residue side chains), or to both. The degree and type of protein glycosylation is specific to the identity of the glycosylated protein, and generally occurs in two stages. In the endoplasmic reticulum, a core oligosaccharide (e.g., dolichol) is bound with residues of a protein; trimming of the core oligosaccharide, addition of other saccharide residues thereto, or both, occur in the endoplasmic reticulum, and especially in the Golgi apparatus. Following modification within the Golgi apparatus, glycosylated proteins are conducted to their ultimate cellular destinations or secreted. Aberrant glycosylation (or non-glycosylation) of proteins and lipids can result in aberrant interaction between the aberrantly glycosylated protein or lipid and a physiological binding partner thereof. Alteration of such binding can be exhibited as a disease or disorder, such as an autoimmune disorder, or a disorder characterized by abnormal (or no) activity of an ordinarily-glycosylated enzyme.

Glycosyl transferases can catalyze biosynthesis of disaccharides, oligosaccharides, and polysaccharides in cells. Thus, glycosyl transferases have a role in storage of carbohydrates (e.g., in production of glycogen) and in physiological production of carbohydrate-based compounds such as glycosaminoglycans, mucopolysaccharides, and extracellular matrix components. Thus, glycosyl transferases are involved in disorders involving aberrant production (or non-production) of storage carbohydrates and other carbohydrate-based compounds. Examples of such disorders include diabetes mellitus, hypoglycemia, arthritis, rheumatism, autoimmune disorders (e.g., systemic lupus erythematosus, Graves' disease, myasthenia gravis, insulin resistance, rheumatoid arthritis, scleroderma, and autoimmune infertility), tumorigenesis, and tumor metastasis.

Numerous glycosyl transferases have been described, and many more are believed to exist. In view of the widespread and critical nature of glycosyl transferase activities in normal and pathological physiological processes, a need exists for identification of further glycosyl transferases. The present invention satisfies this need by providing two novel human glycosyl transferases.

### BRIEF SUMMARY OF THE INVENTION

The present invention is based, in part, on the discovery of novel genes encoding glycosyl transferases, the genes being referred to herein as “47169” and “33935”. The nucleotide sequence of a cDNA encoding 47169 is shown in SEQ ID NO: 1, and the amino acid sequence of a 47169 polypeptide is shown in SEQ ID NO: 2. In addition, the nucleotide sequence of the coding region is depicted in SEQ ID NO: 3. The nucleotide sequence of a cDNA encoding 33935 is shown in SEQ ID NO: 11, and the amino acid sequence of a 33935 polypeptide is shown in SEQ ID NO: 12. In addition, the nucleotide sequence of the coding region is depicted in SEQ ID NO: 13.

Accordingly, in one aspect, the invention features a nucleic acid molecule that encodes a 47169 or 33935 protein or polypeptide, e.g., a biologically active portion of the 47169 protein or a biologically active portion of the 33935 protein. In a preferred embodiment the isolated nucleic acid molecule encodes a polypeptide having the amino acid sequence SEQ ID NO: 2 or SEQ ID NO: 12. In other embodiments, the invention provides isolated 47169 or 33935 nucleic acid molecules having the nucleotide sequence of one of SEQ ID NOs: 1, 3, 11, and 13.

In still other embodiments, the invention provides nucleic acid molecules that have sequences that are substantially identical (e.g., naturally occurring allelic variants) to the nucleotide sequence of one of SEQ ID NOs: 1, 3, 11, and 13. In other embodiments, the invention provides a nucleic acid molecule which hybridizes under stringent hybridization conditions with a nucleic acid molecule having a sequence comprising the nucleotide sequence of one of SEQ ID NOs: 1, 3, 11, and 13, wherein the nucleic acid encodes a full length 47169 or 33935 protein or an active fragment thereof.

In a related aspect, the invention further provides nucleic acid constructs that include a 47169 or 33935 nucleic acid molecule described herein. In certain embodiments, the nucleic acid molecules of the invention are operatively linked to native or heterologous regulatory sequences. Also included are vectors and host cells containing the 47169 or 33935 nucleic acid molecules of the invention, e.g., vectors and host cells suitable for producing 47169 or 33935 nucleic acid molecules and polypeptides.

In another related aspect, the invention provides nucleic acid fragments suitable as primers or hybridization probes for detection of 47169- or 33935-encoding nucleic acids.

In still another related aspect, isolated nucleic acid molecules that are antisense to a 47169- or 33935-encoding nucleic acid molecule are provided.

In another aspect, the invention features 47169 and 33935 polypeptides, and biologically active or antigenic fragments thereof that are useful, e.g., as reagents or targets in assays applicable to treatment and diagnosis of 47169- or 33935-mediated or related disorders (e.g., disorders related to aberrant protein or lipid glycosylation, such as those described herein). In another embodiment, the invention provides 47169 and 33935 polypeptides which are localized in the a cellular membrane (e.g., the cytoplasmic or nuclear membrane or the endoplasmic reticulum) or which are secreted when expressed. In yet another embodiment, the invention provides 47169 and 33935 polypeptides which exhibit the ability to catalyze glycosylation of protein or lipid hydroxyl or amine moieties. Preferred polypeptides are 47169 proteins including at least one glycosyl transferase domain or ricin lectin domain, and preferably having a 47169 activity, e.g., a 47169 activity as described herein. Preferred polypeptides are 47169 proteins including at least one transmembrane domain (which can act as a signal sequence) and at least one glycosyl transferase or ricin lectin domain. Other preferred polypeptides are 33935 proteins including at least one glycosyl transferase domain, and preferably having a 33935 activity, e.g., a 33935 activity as described herein. Preferred polypeptides are 33935 proteins including at least one transmembrane domain (including one which can act as a signal sequence, and preferably including at least four or five transmembrane domains) and at least one glycosyl transferase domain.

In other embodiments, the invention provides 47169 and 33935 polypeptides, e.g., a 47169 polypeptide having the amino acid sequence shown in SEQ ID NO: 2, a 33935 polypeptide having the amino acid sequence shown in SEQ ID NO: 12, an amino acid sequence that is substantially identical to the amino acid sequence shown in either of SEQ ID NOs: 2 and 12, or an amino acid sequence encoded by a nucleic acid molecule having a nucleotide sequence which hybridizes under stringent hybridization conditions to a nucleic acid molecule comprising the nucleotide sequence of any of SEQ ID NOs: 1, 3, 11, and 13, wherein the nucleic acid encodes a full length 47169 protein, a full length 33935 protein, or an active fragment of either of these.

In a related aspect, the invention further provides nucleic acid constructs that include a 47169 or 33935 nucleic acid molecule described herein.

In a related aspect, the invention provides 47169 polypeptides, 33935 polypeptides, or fragments operatively linked to non-47169 or non-33935 polypeptides to form fusion proteins.

In another aspect, the invention features antibodies and antigen-binding fragments thereof, that react with, or more preferably, specifically bind, 47169 or 33935 polypeptides.

In another aspect, the invention provides methods of screening for compounds that modulate the expression or activity of the 47169 or 33935 polypeptides or nucleic acids.

In still another aspect, the invention provides a process for modulating 47169 or 33935 polypeptide or nucleic acid expression or activity, e.g., using the screened compounds. In certain embodiments, the methods involve treatment of conditions related to aberrant activity or expression of the 47169 or 33935 polypeptides or nucleic acids, such as conditions involving aberrant or deficient protein or lipid glycosylation, such as those described herein.

The invention also provides assays for determining the activity of or the presence or absence of 47169 or 33935 polypeptides or nucleic acid molecules in a biological sample, including for disease diagnosis.

In further aspect the invention provides assays for determining the presence or absence of a genetic alteration in a 47169 or 33935 polypeptide or nucleic acid molecule, including for disease diagnosis.

The invention includes a method of modulating the ability of a cell (e.g., a human lung, colon, or breast cell, either normal or tumorous) to affect the glycosylation state of a target lipid or polypeptide. The method comprises modulating (i.e., inhibiting or enhancing) the activity of an enzyme selected from the group consisting of 47169 protein and 33935 protein in the cell. The ability of the cell to affect the glycosylation state of the target is thereby modulated. The activity of the enzyme can be inhibited, for example by inhibiting expression of the gene encoding the enzyme (e.g., using an antisense oligonucleotide), an agent that inhibits the enzyme activity (e.g., an antibody that specifically binds with the enzyme), or enhanced (e.g., by administering an expression vector encoding the enzyme to the cell).

The invention also includes a method for assessing whether a test compound is useful for modulating at least one phenomenon selected from the group consisting of non-covalent binding between a protein and one of a cell, a virus, and another protein; cell signaling; cell differentiation; tumorigenesis; cell adhesion; cell motility; cell-to-cell interaction; cell invasivity; cell proliferation; gene transcription; and an immune response. The method comprises:

a) adding the test compound to a first composition comprising (optionally within a cell) one of
i) a polypeptide that has an amino acid sequence at least 90% identical to SEQ ID NO: 2 and that exhibits a 47169 activity and
ii) a polypeptide that has an amino acid sequence at least 90% identical to SEQ ID NO: 12 and that exhibits a 33935 activity; and

b) comparing the activity in the first composition and in a second composition that is substantially identical to the first composition, except that it lacks the test compound.

A difference in the activity (e.g., glycosyl transferase activity attributable to 47169 or 33935, such as ability to transfer an N-acetylgalactosamine moiety from uridine diphosphate to a hydroxyl moiety of a serine or threonine residue of a protein) in the first and second compositions is an indication that the test compound is useful for modulating the phenomenon.

The invention further includes another method for assessing whether a test compound is useful for modulating at least one of those phenomena. This method comprises:

a) adding the test compound to a composition comprising a cell which comprises a nucleic acid that encodes one of
i) a polypeptide that has an amino acid sequence at least 90% identical to SEQ ID NO: 2 and exhibits a 47169 activity; and
ii) a polypeptide that has an amino acid sequence at least 90% identical to SEQ ID NO: 12 and exhibits a 33935 activity; and

b) comparing the activity in the first composition and in a second composition that is substantially identical to the first composition, except that it lacks the test compound.

A difference in the activity in the first and second compositions is an indication that the test compound is useful for modulating the phenomenon.

In another embodiment, the invention includes a method for identifying a compound useful for modulating at least one of these phenomena. The method comprises:

a) contacting the test compound and a polypeptide (preferably one which exhibits an epitope in common with a polypeptide having the amino acid sequence of one of SEQ ID NOs: 2 and 12) selected from the group consisting of
i) a polypeptide which is encoded by a nucleic acid molecule comprising a portion having a nucleotide sequence which is at least 90% identical to one of SEQ ID NOs: 1, 3, 11, and 13; and
ii) a fragment of a polypeptide having an amino acid sequence selected from the group consisting of SEQ ID NOs: 2 and 12, wherein the fragment comprises at least 25 contiguous amino acid residues of one of SEQ ID NOs: 2 and 12
   or a cell that expresses the polypeptide; and

b) determining whether the polypeptide binds with the test compound. Binding of the polypeptide and the test compound is an indication that the test compound is useful for modulating the phenomenon.

Compounds identified using the preceding methods can be used to make a pharmaceutical composition for modulating at least one of the phenomena. Those pharmaceutical compositions can be administered to a human in order to modulate one or more of the phenomena in the human.

Other features and advantages of the invention will be apparent from the following detailed description, and from the claims.

### BRIEF DESCRIPTION OF THE SEVERAL VIEWS OF THE DRAWINGS

FIG. 1 depicts a cDNA sequence (SEQ ID NO: 1) and predicted amino acid sequence (SEQ ID NO: 2) of human 47169. The methionine-initiated open reading frame of human 47169 (without the 5′- and 3′-non-translated regions) starts at nucleotide 98 of SEQ ID NO: 1, and the coding region (not including the terminator codon; shown in SEQ ID NO: 3) extends through nucleotide 1906 of SEQ ID NO: 1.

FIG. 2 depicts a hydropathy plot of human 47169. Relatively hydrophobic residues are shown above the dashed horizontal line, and relative hydrophilic residues are below the dashed horizontal line. The cysteine residues (cys) are indicated by short vertical lines below the hydropathy trace. The numbers corresponding to the amino acid sequence of human 47169 are indicated. Polypeptides of the invention include fragments which include: all or part of a hydrophobic sequence, i.e., a sequence above the dashed line, e.g., the sequence of about residues 220-235 of SEQ ID NO: 2; all or part of a hydrophilic sequence, i.e., a sequence below the dashed line, e.g., the sequence of about residues 65-110 or 400-415 of SEQ ID NO: 2; a sequence which includes a cysteine residue; or a glycosylation site.

FIG. 3 depicts a cDNA sequence (SEQ ID NO: 11) and predicted amino acid sequence (SEQ ID NO: 12) of human 33935. The methionine-initiated open reading frame of human 33935 (without the 5′- and 3′-non-translated regions) starts at nucleotide 11 of SEQ ID NO: 11, and the coding region (not including the terminator codon; shown in SEQ ID NO: 13) extends through nucleotide 1486 of SEQ ID NO: 11.

FIG. 4 depicts a hydropathy plot of human 33935. Relatively hydrophobic residues are shown above the dashed horizontal line, and relative hydrophilic residues are below the dashed horizontal line. The cysteine residues (cys) are indicated by short vertical lines below the hydropathy trace. The numbers corresponding to the amino acid sequence of human 33935 are indicated. Polypeptides of the invention include fragments which include: all or part of a hydrophobic sequence, i.e., a sequence above the dashed line, e.g., the sequence of about residues 16-40 of SEQ ID NO: 12; all or part of a hydrophilic sequence, i.e., a sequence below the dashed line, e.g., the sequence of about residues 340-360 of SEQ ID NO: 12; a sequence which includes a cysteine residue; or a glycosylation site.

FIG. 5, comprising FIGS. 5A through 5D, is an alignment of the amino acid sequence of 47169 protein (“47169;” SEQ ID NO: 2) with the amino acid sequences of mammalian UDP-N-acetylgalactosamine:polypeptide N-acetylgalactosaminyltransferase (GalNAcT) enzymes, including the human enzyme designated GalNAcT T1 (GENBANK® accession no. U41514; SEQ ID NO: 20), the rat enzyme designated GalNAcT T1 (GENBANK® accession no. U35890; SEQ ID NO: 21), the murine enzyme designated GalNAcT T1 (GENBANK® accession no. U73820; SEQ ID NO: 22), the bovine enzyme designated GalNAcT T1 (GENBANK® accession no. L07780; SEQ ID NO: 23), and the porcine enzyme designated GalNAcT T1 (GENBANK® accession no. D85389; SEQ ID NO: 24). A colon (“:”) indicates manually identified residues at which the amino acid sequence is highly conserved, and a period (“.”) indicates manually identified residues at which the amino acid sequence is less highly conserved.

FIG. 6, comprising FIG. 6A through 6C, is an alignment of the amino acid sequence of 47169 protein (“47169;” SEQ ID NO: 2) with the amino acid sequences of several Caenorhabditis elegans GalNAcT enzymes, including the enzymes designated gly-3 (“gly-3;” GENBANK® accession no. AF031833; SEQ ID NO: 25), gly4 (“gly-4;” GENBANK® accession no. AF031834; SEQ ID NO: 26), gly-5b (“gly-5b;” GENBANK® accession no. AF031836; SEQ ID NO: 27), gly-6a (“gly-6a;” GENBANK® accession no. AF031838; SEQ ID NO: 28), and gly-9 (“gly-9;” GENBANK® accession no. AF031843; SEQ ID NO: 29), in Hagen et al. (1998, J. Biol. Chem 273(14):8268-8277).

### DETAILED DESCRIPTION OF THE INVENTION

In the following two sections, 47169 nucleic acids and proteins are described separately from 33965 nucleic acids and proteins.

47169 Nucleic Acids and Proteins

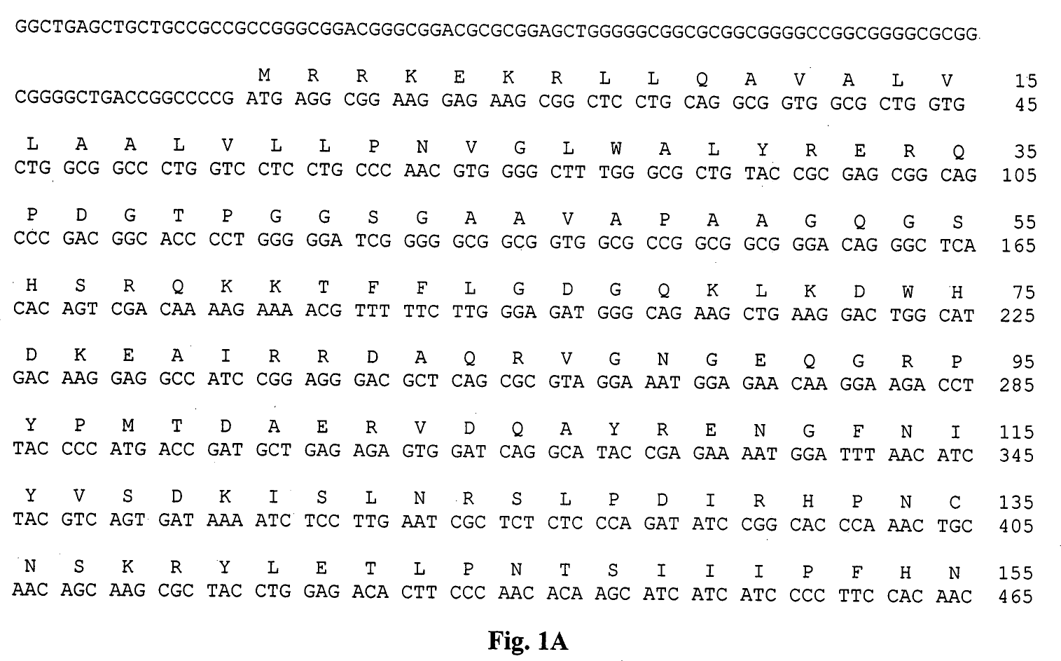

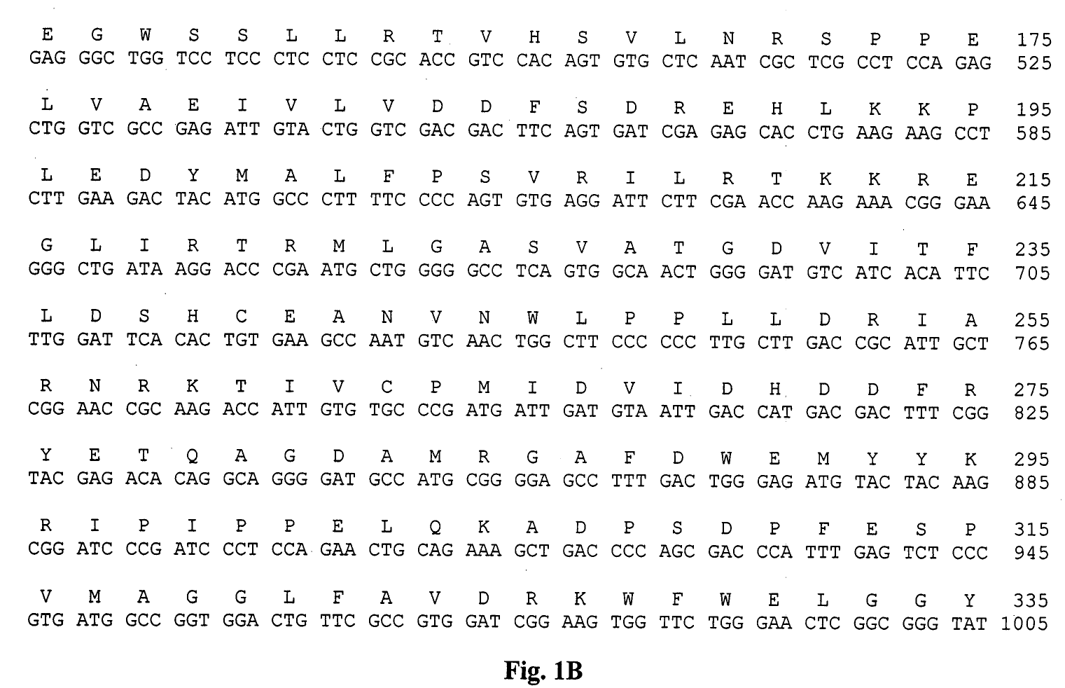

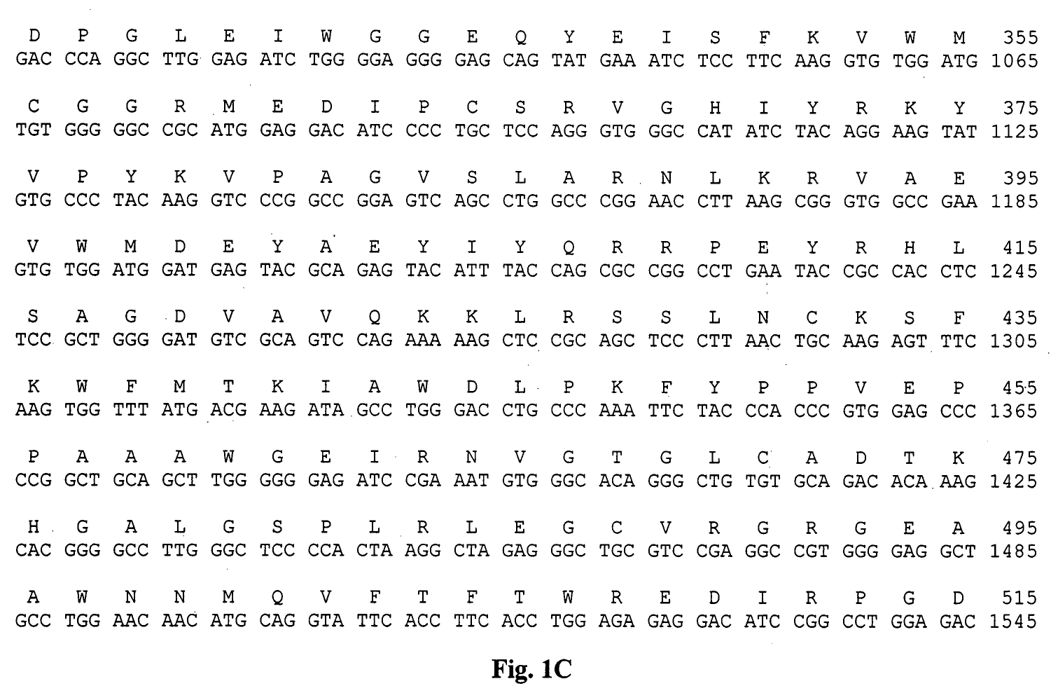

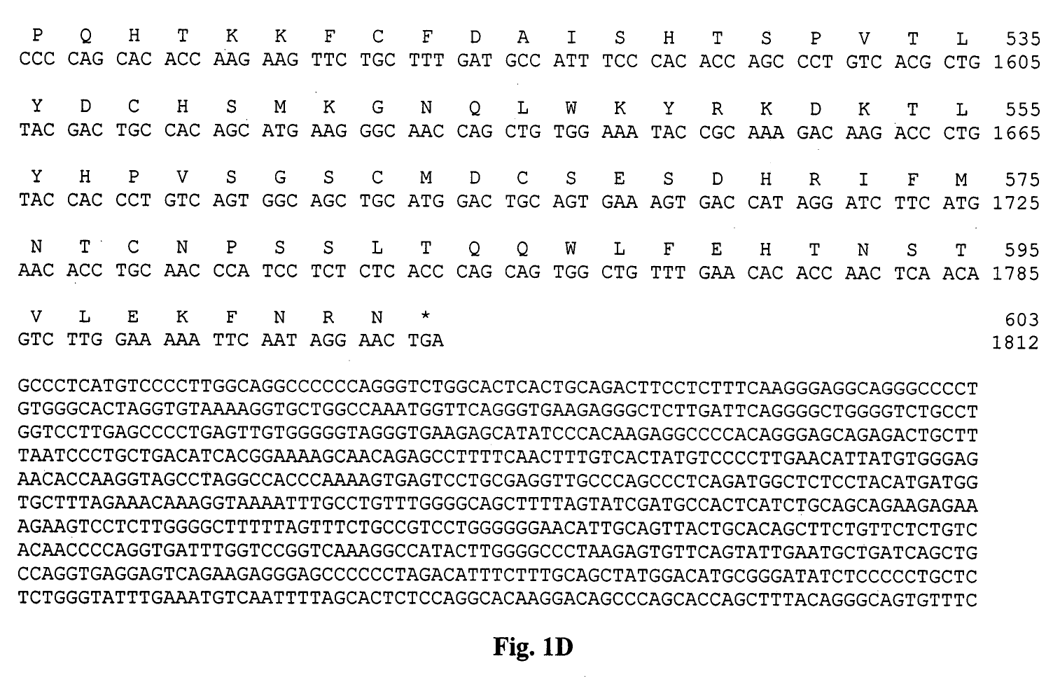

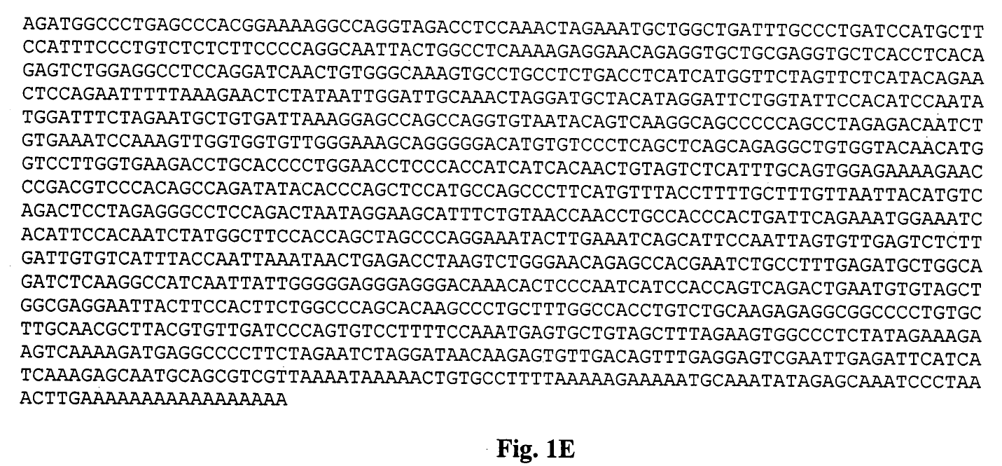

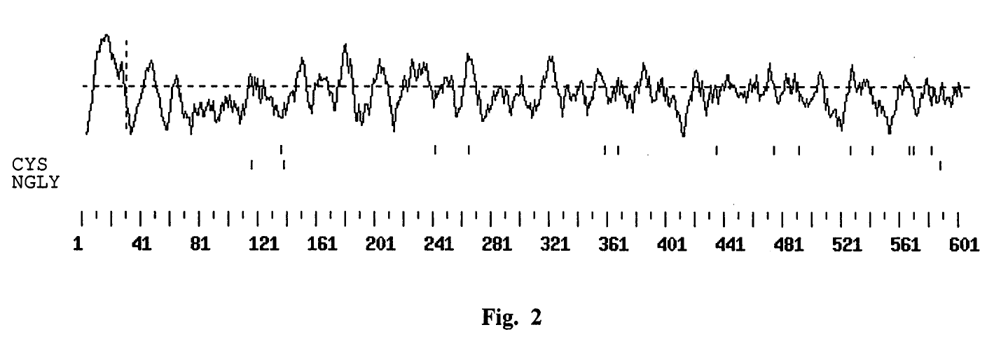

The human 47169 cDNA sequence (FIG. 1; SEQ ID NO: 1), which is approximately 3985 nucleotide residues long including non-translated regions, contains a predicted methionine-initiated coding sequence of about 1809 nucleotide residues, excluding termination codon (i.e., nucleotide residues 98-1906 of SEQ ID NO: 1; also shown in SEQ ID NO: 3). The coding sequence encodes a 603 amino acid residue protein having the amino acid sequence SEQ ID NO: 2.

Human 47169 contains a predicted glycosyl transferase 2 domain (PF00535) at about amino acid residues 148 to 333 and a predicted ricin lectin domain (“Ricin_B_Lectin” domain; PF00652) at about amino acid residues 455 to 590 of SEQ ID NO: 2. A transmembrane domain is predicted at about amino acid residues 8 to 31 of SEQ ID NO: 2. This transmembrane domain can form part of a signal sequence in an embodiment in which the 47169 protein of the invention is a secreted protein lacking amino acid residues 1 to about 29 (i.e., 1-27, 1-28, 1-29, 1-30, or 1-31) of SEQ ID NO: 2. In another embodiment, this transmembrane domain is not cleaved from the mature protein, but instead anchors the protein in a cell membrane (e.g., the cytoplasmic membrane or the membrane surrounding the endoplasmic reticulum or Golgi apparatus).

The human 47169 protein has predicted N-glycosylation sites (Pfam accession number PS00001) at about amino acid residues 124-127, 146-149, and 593-596 of SEQ ID NO: 2; predicted protein kinase C phosphorylation sites (Pfam accession number PS00005) at about amino acid residues 118-120, 137-139, 187-189, 205-207, 211-213, 350-352, 434-436, 506-508, 519-521, and 540-542 of SEQ ID NO: 2; predicted casein kinase II phosphorylation sites (Pfam accession number PS00006) located at about amino acid residues 99-102, 126-129, 172-175, 187-190, 234-237, 238-241, 416-419, 506-509, 534-537, 562-565, 567-570, and 595-598 of SEQ ID NO: 2; predicted tyrosine kinase phosphorylation sites (Pfam accession number PS00007) located at about amino acid residues 285-293, 327-335, and 548-556 of SEQ ID NO: 2; and predicted N-myristoylation sites (Pfam accession number PS00008) at about amino acid residues 38-43, 216-221, 224-229, 319-324, 383-388, 467-472, and 487-492 of SEQ ID NO: 2.

For general information regarding PFAM identifiers, PS prefix and PF prefix domain identification numbers, refer to Sonnhammer et al. (1997, Protein 28:405-420) and http://www.psc.edu/general/software/packages/pfam/pfam.html.

The 47169 protein contains a significant number of structural characteristics in common with members of the glycosyl transferase family. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologues of non-human origin, e.g., glycosyl transferase proteins for any species described in the art. Members of a family can also have common functional characteristics.

A 47169 polypeptide can include a glycosyl transferase domain, such as a glycosyl transferase 2 domain. As used herein, the term “glycosyl transferase 2 domain” refers to a protein domain having an amino acid sequence about 150-200 residues in length, preferably, at least about 170-190 residues, more preferably about 186 or 187 residues and has a bit score for the alignment of the sequence to the glycosyl transferase 2 domain (HMM) of at least 25 or greater, preferably 50 or greater, and more preferably 75 or greater. The glycosyl transferase 2 domain has been assigned the PFAM accession PF00535 (http://genome.wustl.edu/Pfam/html).

A 47169 polypeptide can also, or instead, include a ricin lectin domain. As used herein, the term “ricin lectin domain” refers to a protein domain having an amino acid sequence about 100-200 residues in length, preferably, at least about 125-175 residues, more preferably about 136 or 145 residues and has a bit score for the alignment of the sequence to the ricin lectin domain (HMM) of at least 10 or greater, preferably 20 or greater, and more preferably 25 or greater. The ricin lectin domain has been assigned the PFAM accession PF00652 (http://genome.wustl.edu/Pfam/html).

In a preferred embodiment, a 47169 polypeptide or protein has a glycosyl transferase 2 domain or a region which includes at least about 150-200 (i.e., 170-190, 186, or 187) residues and has at least about 60%, 70%, 80%, 90%, 95%, 99%, or 100% homology with a glycosyl transferase 2 domain, e.g., the glycosyl transferase 2 domain of human 47169 (e.g., residues 148-333 of SEQ ID NO: 2). The 47169 polypeptide or protein also preferably has a ricin lectin domain or a region which includes at least about 100-200 (i.e., 125-175, 136, or 145) residues and has at least about 60%, 70%, 80%, 90%, 95%, 99%, or 100% homology with a ricin lectin domain, e.g., the ricin lectin domain of human 47169 (e.g., residues 455-590 of SEQ ID NO: 2).

To identify the presence of a glycosyl transferase 2 domain profile in a 47169 receptor, the amino acid sequence of the protein is searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for PF00534 and score of 25 (preferably 50) is the default threshold score for determining a hit. For example, using ORFAnalyzer software, a glycosyl transferase 2 domain profile was identified in the amino acid sequence of SEQ ID NO: 2 (e.g., amino acids 148-333 of SEQ ID NO: 2). Accordingly, a 47169 protein having at least about 60-70%, more preferably about 70-80%, or about 80-90% homology with the glycosyl transferase 2 domain profile of human 47169 is within the scope of the invention.

Similarly, to identify the presence of a ricin lectin domain profile in a 47169 receptor, the amino acid sequence of the protein is searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for PF00652 and score of 25 (preferably 50) is the default threshold score for determining a hit. For example, using ORFAnalyzer software, a ricin lectin domain profile was identified in the amino acid sequence of SEQ ID NO: 2 (e.g., amino acids 455-590 of SEQ ID NO: 2). Accordingly, a 47169 protein having at least about 60-70%, more preferably about 70-80%, or about 80-90% homology with the ricin lectin domain profile of human 47169 is within the scope of the invention.

Occurrence in 47169 protein of a ricin lectin domain is an indication that the protein is able to bind specifically with one or more carbohydrate-containing epitopes of a glycosylated protein or lipid. It is recognized that amino acid sequence variation in the ricin lectin domain can alter or abolish the carbohydrate-binding specificity of 47169 protein, and that sequence variation in this region should be minimized unless alteration of the binding specificity is desired. Occurrence of the ricin lectin binding domain is also an indication that 47169 protein can associate or bind specifically with one or more target cells or tissues upon which it normally exerts its physiological effects (e.g., it can bind with the surface of a tissue upon which it exerts glycosyl transferase activity). This observation indicates that 47169 protein, or a ricin lectin domain-containing fragment thereof, can be used to target molecules to the same cells or tissues.

In one embodiment, a 47169 protein includes at least one transmembrane domain which can, in certain embodiments, act as a protein secretion signal sequence. As used herein, the term “transmembrane domain” includes an amino acid sequence of about 5 amino acid residues in length that spans the plasma membrane. More preferably, a transmembrane domain includes about at least 10, 15, 20 or 22 amino acid residues and spans a membrane. Transmembrane domains are rich in hydrophobic residues, and typically have an alpha-helical structure. In a preferred embodiment, at least 50%, 60%, 70%, 80%, 90%, or 95% or more of the amino acids of a transmembrane domain are hydrophobic, e.g., leucines, isoleucines, tyrosines, or tryptophans. Transmembrane domains are described in, for example, htto://pfam.wustl.edu/cgi-bin/getdesc?name=7tm-1, and Zagotta W. N. et al. (1996, Annu. Rev. Neurosci. 19: 235-263), the contents of which are incorporated herein by reference. Amino acid residues 8 to about 31 of SEQ ID NO: 2 comprise a transmembrane domain in a 47169 protein. In one embodiment, the portion of 47169 protein on the carboxyl terminal side of this transmembrane region (i.e., about residues 28-603, 29-603, 30-603, 31-603, or 32-603 of SEQ ID NO: 2) is secreted. In a second embodiment, the transmembrane region is embedded in a cell membrane (e.g., the nuclear membrane or the cytoplasmic membrane), and the carboxyl terminal portion of the protein is oriented on the non-cytoplasmic face of the membrane. In a third embodiment, the transmembrane region is embedded in the cytoplasmic membrane, and the carboxyl terminal portion of the protein is oriented on the cytoplasmic side of a cellular membrane. In another embodiment, the transmembrane region directs secretion of the protein into a cellular compartment (e.g., into the nucleus, into a vacuole, or into a mitochondrion).

In one embodiment of the invention, a 47169 polypeptide includes at least one glycosyl transferase 2 domain, at least one ricin lectin domain, or at least one of each of these domains. In another embodiment, the 47169 polypeptide includes at least a glycosyl transferase 2 domain, a ricin lectin domain, and at least one transmembrane domain, preferably located at or near the amino terminus of the protein. The 47169 molecules of the present invention can further include one or more of the N-glycosylation, cAMP-/cGMP-dependent protein kinase phosphorylation, protein kinase C phosphorylation, casein kinase II phosphorylation, tyrosine kinase phosphorylation, and N-myristoylation described herein, and preferably comprises most or all of them.

Occurrence of both the ricin lectin binding domain and the glycosyl transferase 2 domain in 47169 protein is an indication that 47169 protein is involved in modulating the glycosylation of polypeptides, lipids, or both. 47169 protein can catalyze the initial step of glycosylation of a polypeptide or lipid (e.g., transfer of an N-acetylglucosaminyl residue from UDP-N-acetylglucosamine to a serine or threonine residue of a protein). 47169 protein can also catalyze addition of carbohydrate residues to already-glycosylated polypeptides or lipids. 47169 can catalyze removal of one or more carbohydrate residues from a glycosylated polypeptide or lipid.

The amino acid sequence of the four residues at the amino terminus the four residues at the carboxyl terminus of 47169 protein indicate that the protein can be directed to the endoplasmic reticulum and Golgi apparatus of cells in which it is expressed. This observation indicates that 47169 can modulate glycosylation of protein that occurs in these two cellular compartments. Like other GalNAcT enzymes, 47169 can exist in at least two alternative forms. In one form, the protein is anchored in the membrane of the compartment by the transmembrane region that occurs at about residues 8 to 31 of SEQ ID NO: 2, and the majority of the protein (i.e., about residues 32-603) are situated within the lumen of the compartment. In another form, the protein is cleaved on the lumenal side of the compartment membrane, yielding a glycosyltransferase enzyme that is suspended within the compartment and that can be secreted from the cell upon merger of the compartment membrane with the cell membrane. In either form, 47169 protein can modulate glycosylation of polypeptides and lipids that occur in one or both of the endoplasmic reticulum and the Golgi apparatus. Upon merger of one of these compartments with the cellular membrane and release of compartmental contents into the extracellular milieu, the membrane-bound form of 47169 protein can continue to modulate glycosylation of polypeptides and lipids that are accessible to the cell membrane, and the secreted form of 47169 protein can modulate glycosylation of polypeptides and lipids that are in fluid communication with the extracellular milieu.

The 47169 gene appears to be expressed ubiquitously, or at least in a wide variety of tissues. 47169 expression could be detected in all tissues tested by in situ hybridization or by real time PCR-based experiments. Significantly higher levels of expression were observed in lung (2-15×), breast (2-30×), and colon (2-10×) tumor tissues, relative to the corresponding normal (i.e., non-tumorous) tissues. Significantly higher levels of expression were also observed in ovarian and liver tumor samples, relative to the corresponding normal tissues. These observations indicate that expression of 47169 is enhanced in many tumors of endothelial origin. However, because expression of 47169 did not appear to be significantly enhanced in prostate tumor samples that were tested, relative to normal prostate samples, 47169 does not appear to be over-expressed in all tumors of endothelial origin. 47169 was also highly expressed in metastasized tumor cells (e.g., metastasized colon cells found in liver tissue). These observations indicate that aberrant expression, activity, or both, of 47169 is associated with one or more of tumorigenesis, tumor growth, tumor cell proliferation, and metastasis. Thus, 47169 molecules can be used to inhibit, prevent, prognosticate, diagnose, alleviate, or reverse any of tumorigenesis, tumor growth, tumor invasion of surrounding tissues, tumor cell proliferation, and metastasis.

These observations are also supported by observations in the literature that other GalNAcT enzymes are differentially expressed in colon tumor tissues, relative to normal colon tissues (e.g., Kohsaki et al., 2000, J. Gastroenterol. 35(11):840-848; Ten Hagen et al., 1999, J. Biol. Chem. 274(39):27867-27874; Sutherline et al., 1997, Cancer Res. 57(21):4744-4748). As described by others, different GalNAcT enzymes exhibit different patterns of tissue expression and different substrate specificity. For example, the human GalNAcT designated GalNAc-T3 is preferentially expressed in differentiated adenocarcinoma cells (Sutherlin et al., 1997, Cancer Res. 57(21):4744-4748). Overexpression of 47169 was more apparent in highly invasive colon adenocarcinomas than in colonic adenocarcinoma metastases found in liver tissue. Similarly, overexpression of 47169 was more apparent in invasive breast ductal and lobular carcinomas than in benign breast tumor tissues. These observations indicate that 47169 can have a particularly pronounced role in tumor growth and infiltration (or invasion) of tumor tissue into surrounding tissues.

High levels of expression detected in vascular endothelial and smooth muscle tissues indicate that 47169 can have a role in normal vascular function. This observation conversely indicates that aberrant expression, activity, or both, of 47169 can be associated with vascular disorders such as atherosclerosis, arteriosclerosis, abnormal blood coagulation (including stroke), coronary artery disease, bacterial and viral infections, aberrant inflammation, pain disorders associated with aberrant vascular permeability, and disorders associated with aberrant ability of leukocytes to adhere to and translocate across vascular tissues.

Relatively high levels of expression of 47169 in breast tissue indicate that aberrant expression, activity, or both, of 47169 (i.e., the secreted form) can alter the composition and properties of breast milk, such as colostrum. The ability of 47169 protein secreted into breast milk to modulate glycosylation of polypeptides and lipids confers this ability to at least the digestive tract of an infant that ingests the milk. 47169 glycosylation modulating activity in the infant's digestive tract can alter the glycosylation state of cells lining the infant's digestive tract (e.g., altering the cell's interactions with one another or with bacteria or viruses in the tract), and can also modulate the glycosylation of bacteria that occur in the tract (e.g., altering their ability to interact with cells of the digestive tract). Thus, modulating expression, activity, or both, of 47169 in breast milk or breast tissue can inhibit, prevent, prognosticate, diagnose, alleviate, or reverse one or more disorders that affect the infant digestive system. Examples of these disorders include nutritional disorders, bacterial and viral infections (e.g., diarrhea), and colic.

Experiments performed with cells having cell cycles synchronized using nocodazole indicated that expression of 47169 is enhanced during the G2/M phase of the cell cycle, which is associated with division of a tetraploid cell into a pair of diploid cells. This observation indicates that 47169 has a role in disorders associated with aberrant division and proliferation of cells. Examples of these disorders include tumorigenesis, tumor cell proliferation, tumor invasion of surrounding tissues, aberrant angiogenesis, and vascular restenosis.

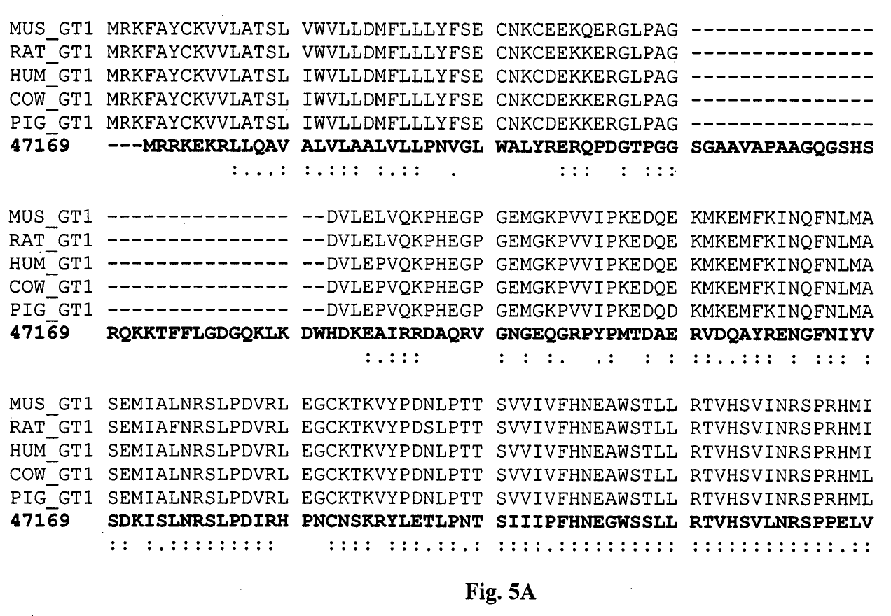

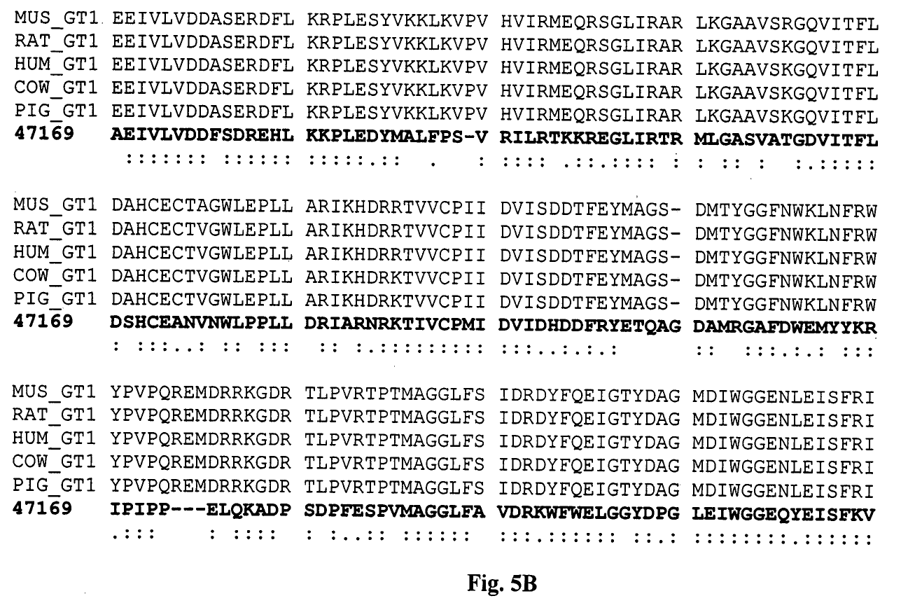

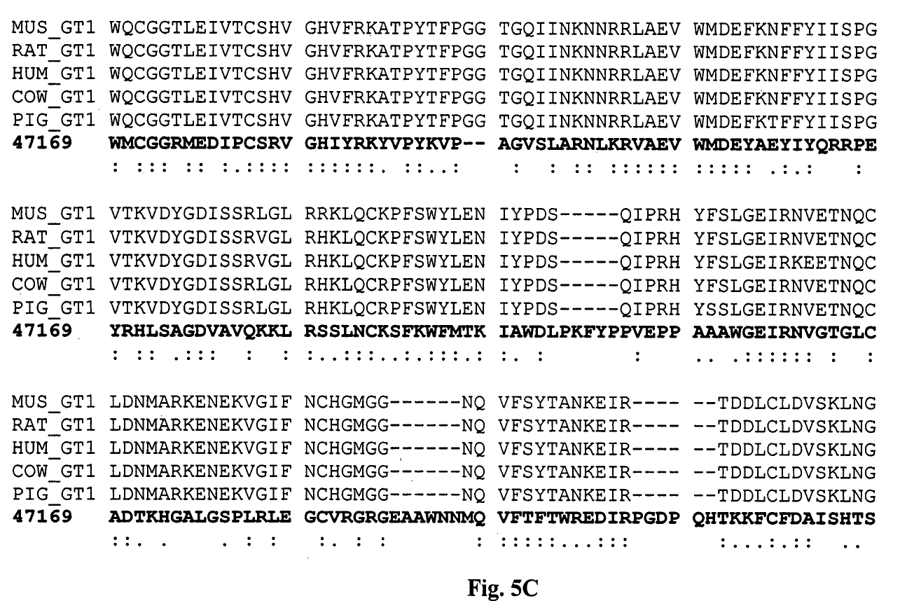

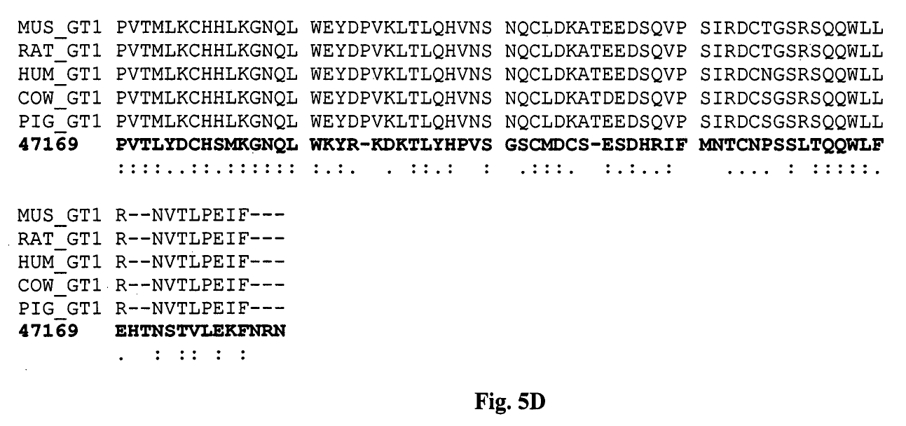

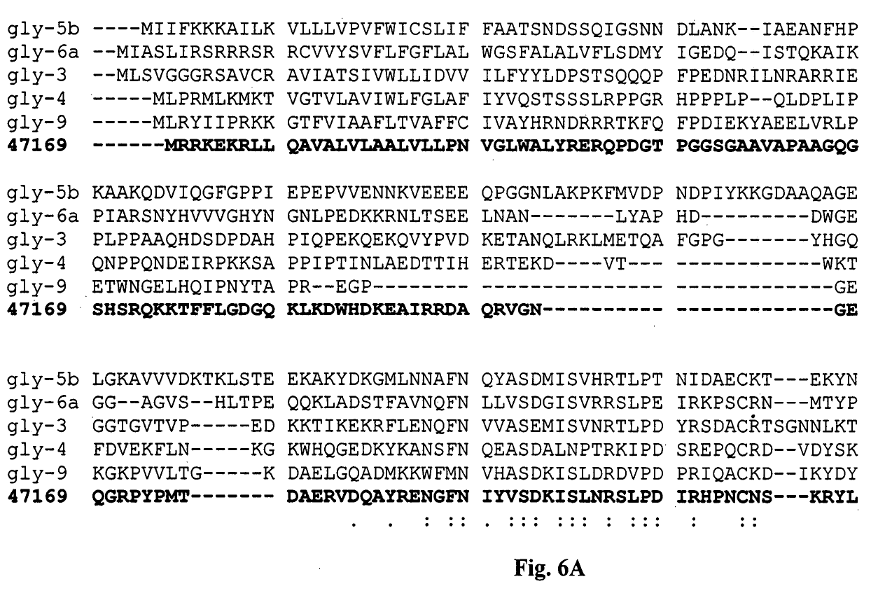

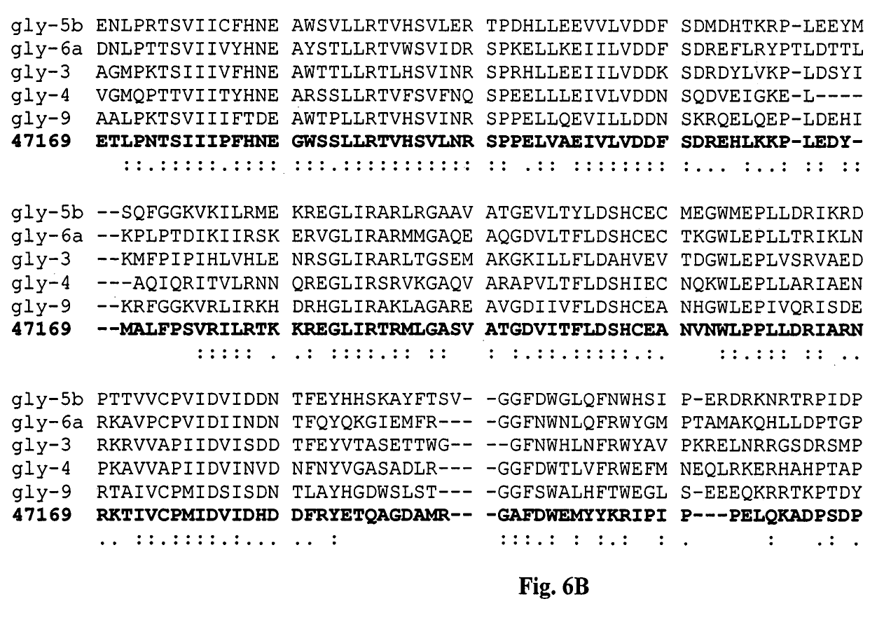

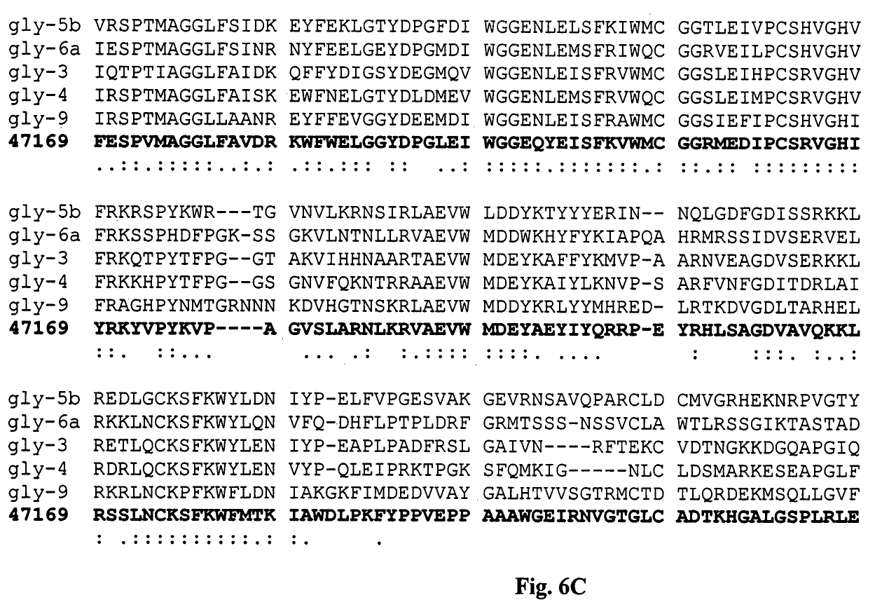

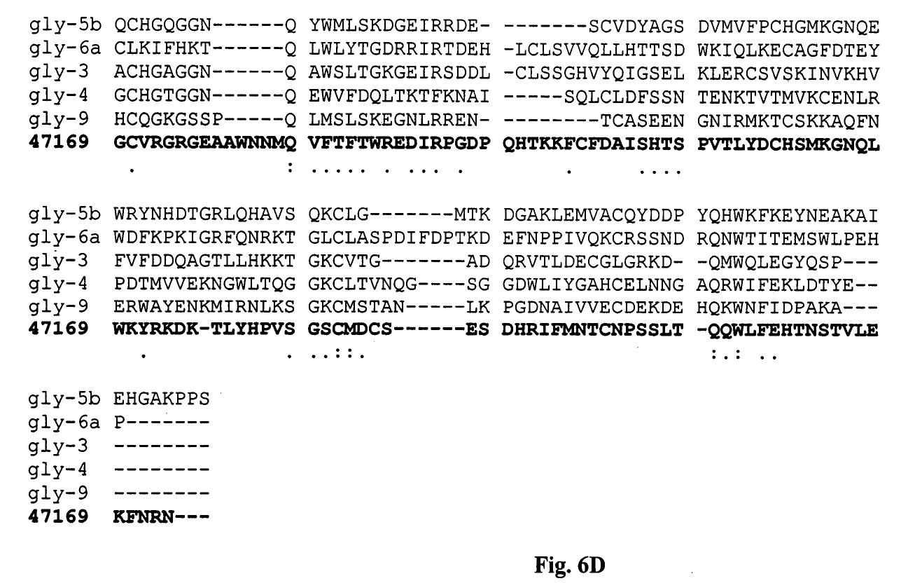

As indicated in FIGS. 5 and 6, 47169 protein exhibits significant amino acid sequence homology with mammalian and Caenorhabditis elegans GalNAcT enzymes. These observations indicate that 47169 protein can catalyze glycosylation of polypeptides (e.g., addition of an N-acetylgalactosamine or other carbohydrate moiety at the hydroxyl moiety of a protein serine or threonine residue).

33935 Nucleic Acids and Proteins

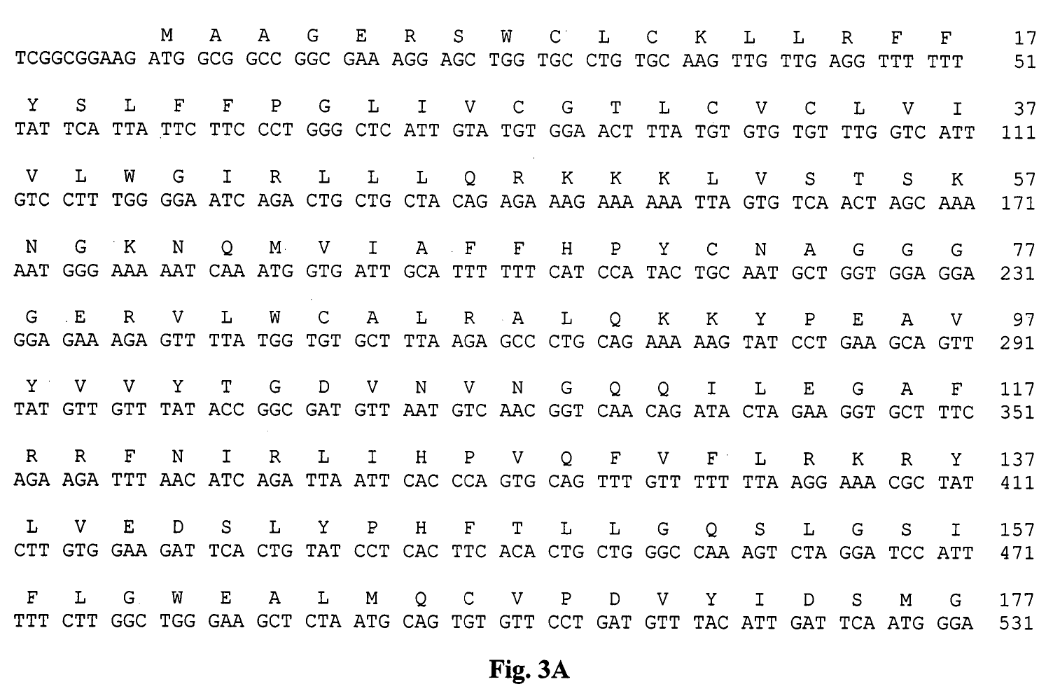

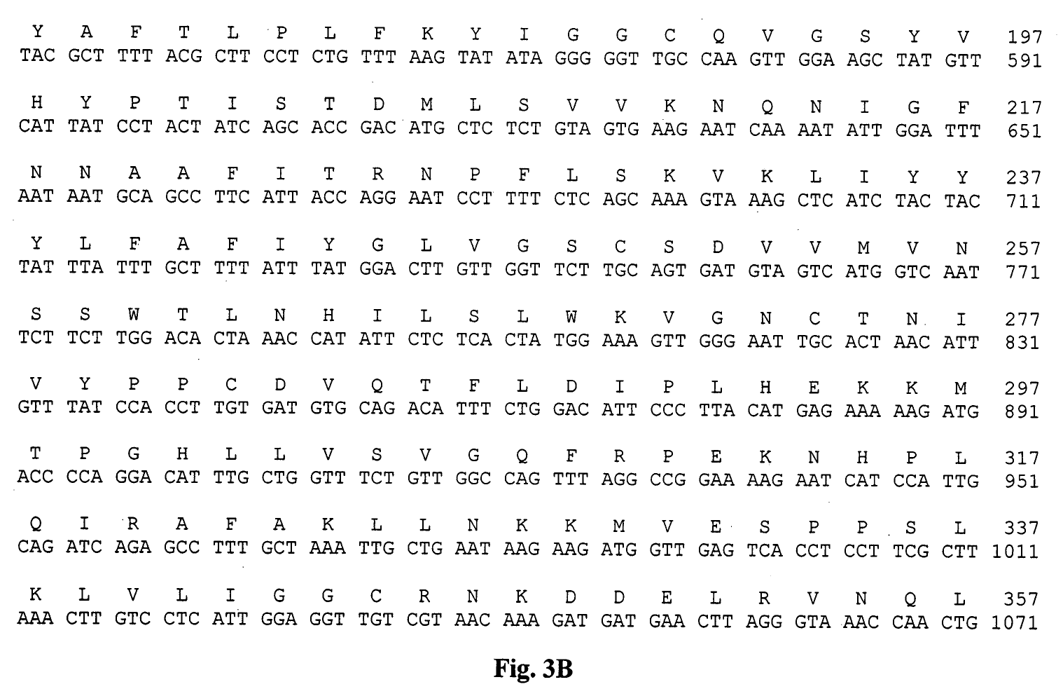

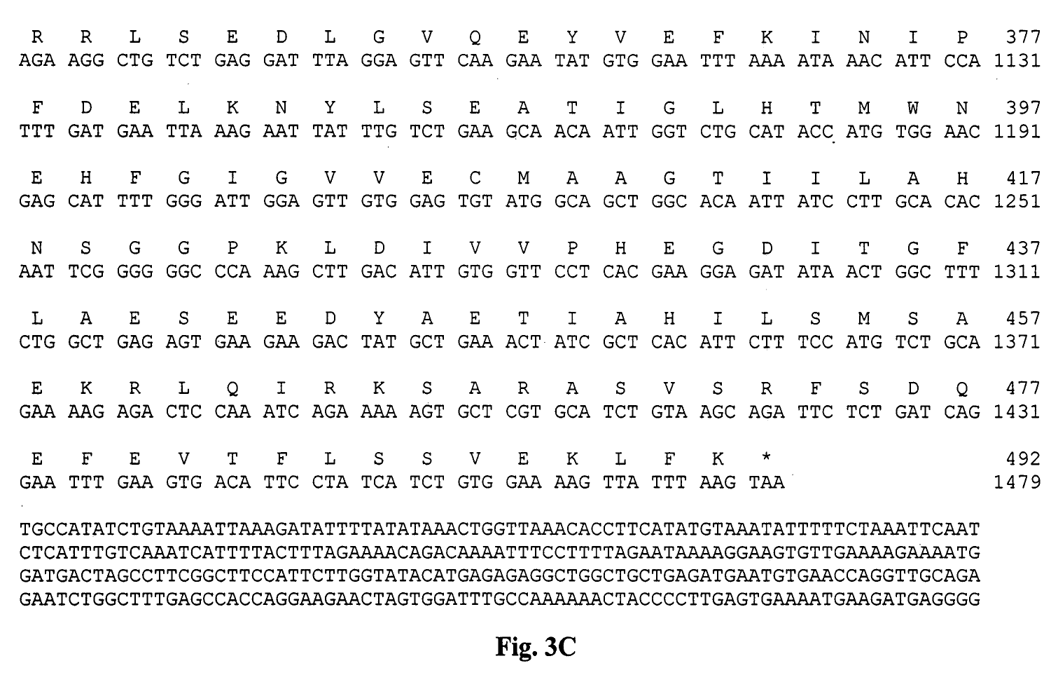

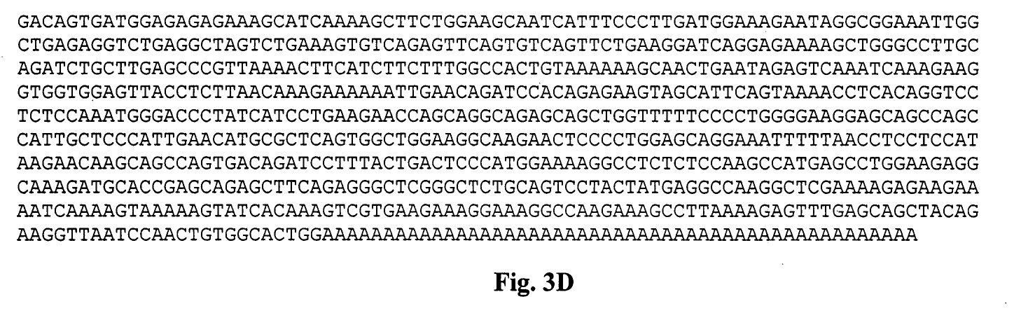

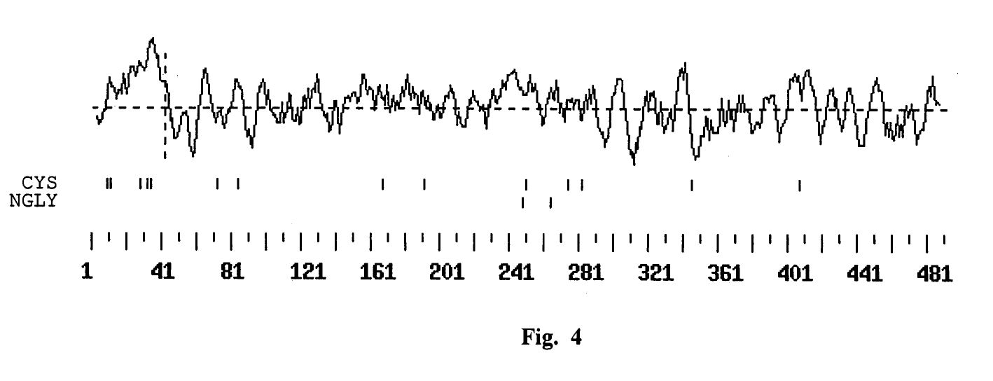

The human 33935 cDNA sequence (FIG. 3; SEQ ID NO: 11), which is approximately 2590 nucleotide residues long including non-translated regions, contains a predicted methionine-initiated coding sequence of about 1476 nucleotide residues, excluding termination codon (i.e., nucleotide residues 11-1486 of SEQ ID NO: 11; also shown in SEQ ID NO: 13). The coding sequence encodes a 492 amino acid residue protein having the amino acid sequence SEQ ID NO: 12.

Human 33935 contains a predicted glycosyl transferase 1 domain (PF00535) at about amino acid residues 286-470 of SEQ ID NO: 12. Transmembrane domains are predicted at about amino acid residues 16-40, 143-161, 176-197, 234-256, and 400-416 of SEQ ID NO: 12. The first of these (residues 16-40) can, in some embodiments, act as a signal sequence that is cleaved (e.g., between residues 39 and 40, between residues 40 and 41, between residues 41 and 42, between residues 42 and 43, or between residues 43 and 44 of SEQ ID NO: 12) following translocation of all or part of 33935 protein across a cell membrane (e.g., the cytoplasmic or nuclear membrane, or the endoplasmic reticulum/Golgi apparatus). In other embodiments, this sequence is not cleaved, and the transmembrane region predicted at residues 16-40 of SEQ ID NO: 12 remains embedded in a cell membrane.

The human 33935 protein has predicted N-glycosylation sites (Pfam accession number PS00001) at about amino acid residues 257-260 and 273-276 of SEQ ID NO: 12; predicted cAMP-/cGMP-dependent protein kinase phosphorylation sites (Pfam accession number PS00004) at about amino acid residues 295-298 and 358-361 of SEQ ID NO: 12; predicted protein kinase C phosphorylation sites (Pfam accession number PS00005) at about amino acid residues 55-57, 336-338, and 466-468 of SEQ ID NO: 12; predicted casein kinase II phosphorylation sites (Pfam accession number PS00006) located at about amino acid residues 249-252, 286-289, 441-444, 475-478, and 485-488 of SEQ ID NO: 12; predicted tyrosine kinase phosphorylation sites (Pfam accession number PS00007) located at about amino acid residues 91-98 and 136-144 of SEQ ID NO: 12; and predicted N-myrstoylation sites (Pfam accession number PS00008) at about amino acid residues 24-29, 151-156, 190-195, 245-250, 272-277, 343-348, and 403-408 of SEQ ID NO: 12.

For general information regarding PFAM identifiers, PS prefix and PF prefix domain identification numbers, refer to Sonnhammer et al. (1997, Protein 28:405-420) and http://www.psc.edu/general/software/packages/pfam/pfam.html.

The 33935 protein contains a significant number of structural characteristics in common with members of the glycosyl transferase family.

A 33935 polypeptide can include a glycosyl transferase 1 domain. As used herein, the term “glycosyl transferase 1 domain” refers to a protein domain having an amino acid sequence about 150-200 residues in length, preferably, at least about 170-190 residues, more preferably about 185, or 191 residues and has a bit score for the alignment of the sequence to the glycosyl transferase 1 domain (HMM) of at least 25 or greater, preferably 50 or greater, and more preferably 75 or greater. The glycosyl transferase 1 domain has been assigned the PFAM accession PF00535 (http://genome.wustl.edu/Pfam/html).

In a preferred embodiment, a 33935 polypeptide or protein has a glycosyl transferase 1 domain or a region which includes at least about 150-200, more preferably about 170-190, 185, or 191 amino acid residues and has at least about 60%, 70%, 80%, 90%, 95%, 99%, or 100% homology with a glycosyl transferase 1 domain, e.g., the glycosyl transferase 1 domain of human 33935 (e.g., residues 286-470 of SEQ ID NO: 12).

To identify the presence of a glycosyl transferase 1 domain profile in a 33935 receptor, the amino acid sequence of the protein is searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for PF00535 and score of 25 (preferably 50) is the default threshold score for determining a hit. For example, using ORFAnalyzer software, a glycosyl transferase 1 domain profile was identified in the amino acid sequence of SEQ ID NO: 12 (e.g., amino acids 286-470 of SEQ ID NO: 12). Accordingly, a 33935 protein having at least about 60-70%, more preferably about 70-80%, or about 80-90% homology with the glycosyl transferase 1 domain profile of human 33935 is within the scope of the invention.

In one embodiment, a 33935 protein includes at least one transmembrane domain, and preferably includes four or five transmembrane domains. As used herein, the term “transmembrane domain” includes an amino acid sequence of about 5 amino acid residues in length that spans the plasma membrane. More preferably, a transmembrane domain includes about at least 10, 15, 20 or 22 amino acid residues and spans a membrane. Transmembrane domains are rich in hydrophobic residues, and typically have an alpha-helical structure. In a preferred embodiment, at least 50%, 60%, 70%, 80%, 90%, or 95% or more of the amino acids of a transmembrane domain are hydrophobic, e.g., leucines, isoleucines, tyrosines, or tryptophans. Transmembrane domains are described in, for example, htto://pfam.wustl.edu/cgi-bin/getdesc?name=7tm-1, and Zagotta W. N. et al. (1996, Annu. Rev. Neurosci. 19: 235-263), the contents of which are incorporated herein by reference. Amino acid residues 16-40, 143-161, 176-197, 234-256, and 400-416 of SEQ ID NO: 12 are predicted to comprise transmembrane domains in 33935 protein. In one embodiment, the regions corresponding to about residues 41-142, 198-233, and 417-492 of SEQ ID NO: 12 are oriented on the cytoplasmic side of a cellular membrane (e.g., the nuclear or cytoplasmic membrane or the endoplasmic reticulum) and the regions corresponding to about residues 162-175 and 257-399 of SEQ ID NO: 12 are oriented on the non-cytoplasmic side of the same membrane. In another embodiment, the orientations of these groups of regions are reversed.

In one embodiment of the invention, a 33935 polypeptide includes at least one glycosyl transferase 1 domain. In another embodiment, the 33935 polypeptide includes at least one glycosyl transferase 1 domain and at least one (preferably four or five) transmembrane domain. The 33935 molecules of the present invention can further include one or more of the N-glycosylation, cAMP-/cGMP-dependent protein kinase phosphorylation, protein kinase C phosphorylation, casein kinase II phosphorylation, tyrosine kinase phosphorylation, and N-myristoylation sites described herein, and preferably comprises most or all of them.

Occurrence of a glycosyl transferase 1 domain and multiple transmembrane domains in 33935 protein indicates that 33935 protein can modulate glycosylation of membrane-associated proteins (e.g., membrane bound proteins and integral membrane proteins) and lipids. For example, 33935 can modulate glycosylation (e.g., existence of O-linked N-acetylgalactosamine residues at serine and threonine residues) of mucin proteins. 33935 can also modulate glycosylation of lipids such as sphingolipids and other glycosyl ceramides and gangliosides (e.g., mono-, di-, and tri-sialo-gangliosides). The carboxy-terminal amino acid residues of 33935 protein indicate that 33935 protein can occur in the membrane surrounding the endoplasmic reticulum and the Golgi apparatus (and in the cytoplasmic membrane following fusion of Golgi or endoplasmic reticulum vesicles therewith). Thus, 33935 protein can modulate the glycosylation state of both cell surface proteins and lipids and secreted proteins. Aberrant expression, activity, or both of 33936 can have physiological effects like those described herein for 47169.

In the remainder of this specification, the designation “47169/33935” indicates that the molecule, disorder, or property modified by this designation includes, collectively or alternatively, both molecules, disorders, or properties corresponding to 47169 and molecules, disorders, or properties corresponding to 33935.

Because the 47169/33935 polypeptides of the invention can modulate 47169/33935-mediated activities, they can be used to develop novel diagnostic and therapeutic agents for 47169/33935-mediated or related disorders, as described below.

As used herein, a “47169/33935 activity,” “biological activity of 47169/33935,” or “functional activity of 47169/33935,” refers to an activity exerted by a 47169/33935 protein, polypeptide or nucleic acid molecule on, for example, a 47169/33935-responsive cell or on a 47169/33935 substrate (e.g., a protein substrate) as determined in vivo or in vitro. In one embodiment, a 47169/33935 activity is a direct activity, such as association with a 47169/33935 target molecule. A “target molecule” or “binding partner” of a 47169/33935 protein is a molecule (e.g., a protein or nucleic acid) with which the 47169/33935 protein binds or interacts in nature. In an exemplary embodiment, such a target molecule is a 47169/33935 receptor. A 47169/33935 activity can also be an indirect activity, such as a cellular signaling activity mediated by interaction of the 47169/33935 protein with a 47169/33935 receptor.

The 47169/33935 molecules of the present invention are predicted to have similar biological activities as glycosyl transferase family members. For example, the 47169/33935 proteins of the present invention can have one or more of the following activities:
(1) catalyzing formation of a covalent bond between a carbohydrate (i.e., saccharide) moiety and a hydroxyl or amino moiety of a protein (e.g., a serine, threonine, or tyrosine side chain of an amino acid residue of a protein) or lipid;
(2) facilitating non-covalent binding between a protein and a cell, virus, or other protein;
(3) modulating cell signaling;
(4) modulating cell differentiation;
(5) modulating tumorigenesis;
(6) modulating cell adhesion;
(7) modulating cell motility;
(8) modulating a cell-to-cell interaction;
(9) modulating cell invasivity (e.g., metastatic or extravasative capacity of an individual cell or invasion of surrounding tissues by a tumor);
(10) modulating cell proliferation (e.g., proliferation of breast, lung, liver, or colon tumor cells or proliferation of vascular endothelial cells);
(11) modulating gene transcription; and
(12) modulating an immune response (e.g., an autoimmune response or a response to a pathogen).
   Thus, 47169/33935 molecules described herein can act as novel diagnostic targets and therapeutic agents for prognosticating, diagnosing, preventing, inhibiting, alleviating, or reversing (e.g., curing) glycosyl transferase-related disorders, such as those disclosed herein.

Other activities, as described below, include the ability to modulate function, survival, morphology, proliferation and/or differentiation of cells of tissues in which 47169/33935 molecules are expressed. Thus, the 47169/33935 molecules can act as novel diagnostic targets and therapeutic agents for controlling disorders involving aberrant activities of these cells.

The 47169/33935 molecules can also act as novel diagnostic targets and therapeutic agents for controlling cellular proliferative and/or differentiative disorders (e.g., hematopoietic neoplastic disorders, carcinoma, sarcoma, metastatic disorders or hematopoietic neoplastic disorders, such as leukemias). A metastatic tumor can arise from a multitude of primary tumor types, including but not limited to those of prostate, colon, lung, breast and liver origin.

As used herein, the terms “cancer,” “hyperproliferative” and “neoplastic” refer to cells having the capacity for autonomous growth, i.e., an abnormal state or condition characterized by rapidly proliferating cell growth. Hyperproliferative and neoplastic disease states can be categorized as pathologic, i.e., characterizing or constituting a disease state, or can be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. The disorders can arise from poorly differentiated acute leukemias, e.g., erythroblastic leukemia and acute megakaryoblastic leukemia. Exemplary myeloid disorders include, but are not limited to, acute promyeloid leukemia, acute myelogenous leukemia and chronic myelogenous leukemia (reviewed in Vaickus, 1991, Crit. Rev. Oncol./Hemotol. 11:267-297); lymphoid malignancies include, but are not limited to acute lymphoblastic leukemia which includes B-lineage ALL and T-lineage ALL, chronic lymphocytic leukemia, prolymphocytic leukemia, hairy cell leukemia, and Waldenstrom's macroglobulinemia. Additional forms of malignant lymphomas include, but are not limited to non-Hodgkin lymphoma and variants thereof, peripheral T cell lymphomas, adult T cell leukemia/lymphoma, cutaneous T-cell lymphoma, large granular lymphocytic leukemia, Hodgkin's disease and Reed-Sternberg disease.

The 47169/33935 protein, fragments thereof, and derivatives and other variants of the sequence in SEQ ID NOs: 2 and 12 thereof are collectively referred to as “polypeptides or proteins of the invention” or “47169/33935 polypeptides or proteins”. Nucleic acid molecules encoding such polypeptides or proteins are collectively referred to as “nucleic acids of the invention” or “47169/33935 nucleic acids.” 47169/33935 molecules refer to 47169/33935 nucleic acids, polypeptides, and antibodies.

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules that are separated from other nucleic acid molecules that are present in the natural source of the nucleic acid. For example, with regards to genomic DNA, the term “isolated” includes nucleic acid molecules that are separated from the chromosome with which the genomic DNA is naturally associated. Preferably, an “isolated” nucleic acid is free of sequences that naturally flank the nucleic acid (i.e., sequences located at the 5′- and/or 3′-ends of the nucleic acid) in the genomic DNA of the organism from which the nucleic acid is derived. For example, in various embodiments, the isolated nucleic acid molecule can contain less than about 5 kilobases, 4 kilobases, 3 kilobases, 2 kilobases, 1 kilobase, 0.5 kilobase or 0.1 kilobase of 5′- and/or 3′-nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under stringent conditions” describes conditions for hybridization and washing. Stringent conditions are known to those skilled in the art and can be found in available references (e.g., Current Protocols in Molecular Biology, John Wiley & Sons, N.Y., 1989, 6.3.1-6.3.6). Aqueous and non-aqueous methods are described in that reference and either can be used. A preferred example of stringent hybridization conditions are hybridization in 6× sodium chloride/sodium citrate (SSC) at about 45° C., followed by one or more washes in 0.2×SSC, 0.1% (w/v) SDS at 50° C. Another example of stringent hybridization conditions are hybridization in 6×SSC at about 45° C., followed by one or more washes in 0.2×SSC, 0.1% (w/v) SDS at 55° C. A further example of stringent hybridization conditions are hybridization in 6×SSC at about 45° C., followed by one or more washes in 0.2×SSC, 0.1% (w/v) SDS at 60° C. Preferably, stringent hybridization conditions are hybridization in 6×SSC at about 45° C., followed by one or more washes in 0.2×SSC, 0.1% (w/v) SDS at 65° C. Particularly preferred stringency conditions (and the conditions that should be used if the practitioner is uncertain about what conditions should be applied to determine if a molecule is within a hybridization limitation of the invention) are 0.5 molar sodium phosphate, 7% (w/v) SDS at 65° C., followed by one or more washes at 0.2×SSC, 1% (w/v) SDS at 65° C. Preferably, an isolated nucleic acid molecule of the invention that hybridizes under stringent conditions to the sequence of one of SEQ ID NOs: 1, 3, 11, and 13, corresponds to a naturally-occurring nucleic acid molecule.

As used herein, a “naturally-occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules which include an open reading frame encoding a 47169/33935 protein, preferably a mammalian 47169/33935 protein, and can further include non-coding regulatory sequences and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of 47169/33935 protein having less than about 30%, 20%, 10% and more preferably 5% (by dry weight), of non-47169/33935 protein (also referred to herein as a “contaminating protein”), or of chemical precursors or non-47169/33935 chemicals. When the 47169/33935 protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of 47169/33935 (e.g., the sequence of SEQ ID NOs: 1, 3, 11, and 13) without abolishing or, more preferably, without substantially altering a biological activity, whereas an “essential” amino acid residue results in such a change. For example, amino acid residues that are conserved among the polypeptides of the present invention, e.g., those present in the glycosyl transferase 1 and glycosyl transferase 2 domains (i.e., hereafter collectively referred to as a glycosyl transferase domain) and ricin lectin domains are predicted to be particularly non-amenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in a 47169/33935 protein is preferably replaced with another amino acid residue from the same side chain family. Alternatively, in another embodiment, mutations can be introduced randomly along all or part of a 47169/33935 coding sequence, such as by saturation mutagenesis, and the resultant mutants can be screened for 47169/33935 biological activity to identify mutants that retain activity. Following mutagenesis of one of SEQ ID NOs: 1, 3, 11, and 13, the encoded protein can be expressed recombinantly and the activity of the protein can be determined.

As used herein, a “biologically active portion” of a 47169/33935 protein includes a fragment of a 47169/33935 protein that participates in an interaction between a 47169/33935 molecule and a non-47169/33935 molecule. Biologically active portions of a 47169/33935 protein include peptides comprising amino acid sequences sufficiently homologous to or derived from the amino acid sequence of the 47169/33935 protein, e.g., the amino acid sequence shown in one of SEQ ID NOs: 2 and 12, which include less amino acids than the full length 47169/33935 proteins, and exhibit at least one activity of a 47169/33935 protein. Typically, biologically active portions comprise a domain or motif with at least one activity of the 47169/33935 protein, e.g., a domain or motif capable of catalyzing an activity described herein, such as formation of a covalent bond between a saccharide moiety and a protein amino acid residue side chain (e.g., a serine, hydroxylysine, or threonine hydroxyl moiety or an asparagine amino moiety) or between a saccharide moiety and a lipid hydroxyl or amine moiety.

A biologically active portion of a 47169/33935 protein can be a polypeptide that for example, 10, 25, 50, 100, 200, 300, 400, 500, or 600 or more amino acids in length. Biologically active portions of a 47169/33935 protein can be used as targets for developing agents that modulate a 47169/33935-mediated activity, e.g., a biological activity described herein.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence (e.g., when aligning a second sequence to the 47169/33935 amino acid sequence of SEQ ID NOs: 2 and 12, 100 amino acid residues, preferably at least 200, 300, 400, 500, or 600 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino, acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970, J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

The percent identity between two amino acid or nucleotide sequences can be determined using the algorithm of Meyers et al. (1989, CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990, J. Mol. Biol. 215:403-410). BLAST nucleotide searches can be performed with the NBLAST program, score=100, wordlength=12 to obtain nucleotide sequences homologous to 47169/33935 nucleic acid molecules of the invention. BLAST protein searches can be performed with the XBLAST program, score=50, wordlength=3 to obtain amino acid sequences homologous to 47169/33935 protein molecules of the invention. To obtain gapped alignments for comparison purposes, gapped BLAST can be utilized as described in Altschul et al. (1997, Nucl. Acids Res. 25:3389-3402). When using BLAST and gapped BLAST programs, the default parameters of the respective programs (e.g., XBLAST and NBLAST) can be used. See <http://www.ncbi.nlm.nih.gov>.

“Malexpression or aberrant expression,” as used herein, refers to a non-wild-type pattern of gene expression, at the RNA or protein level. It includes: expression at non-wild-type levels, i.e., over- or under-expression; a pattern of expression that differs from wild-type in terms of the time or stage at which the gene is expressed, e.g., increased or decreased expression (as compared with wild-type) at a predetermined developmental period or stage; a pattern of expression that differs from wild-type in terms of decreased expression (as compared with wild-type) in a predetermined cell type or tissue type; a pattern of expression that differs from wild-type in terms of the splicing size, amino acid sequence, post-transitional modification, or biological activity of the expressed polypeptide; a pattern of expression that differs from wild-type in terms of the effect of an environmental stimulus or extracellular stimulus on expression of the gene, e.g., a pattern of increased or decreased expression (as compared with wild-type) in the presence of an increase or decrease in the strength of the stimulus.

“Subject,” as used herein, can refer to a mammal, e.g., a human, or to an experimental or animal or disease model. The subject can also be a non-human animal, e.g., a horse, cow, goat, or other domestic animal.

A “purified preparation of cells,” as used herein, refers to, in the case of plant or animal cells, an in vitro preparation of cells and not an entire intact plant or animal. In the case of cultured cells or microbial cells, it consists of a preparation of at least 10%, and more preferably, 50% of the subject cells.

Various aspects of the invention are described in further detail below.

Isolated Nucleic Acid Molecules

In one aspect, the invention provides, an isolated or purified, nucleic acid molecule that encodes a 47169/33935 polypeptide described herein, e.g., a full-length 47169/33935 protein or a fragment thereof, e.g., a biologically active portion of 47169/33935 protein. Also included is a nucleic acid fragment suitable for use as a hybridization probe, which can be used, e.g., to a identify nucleic acid molecule encoding a polypeptide of the invention, 47169/33935 mRNA, and fragments suitable for use as primers, e.g., PCR primers for the amplification or mutation of nucleic acid molecules.

In one embodiment, an isolated nucleic acid molecule of the invention includes the nucleotide sequence shown in one of SEQ ID NOs: 1 and 11 or a portion of either of these nucleotide sequences. In one embodiment, the nucleic acid molecule includes sequences encoding the human 47169/33935 protein (i.e., “the coding region,” from nucleotide residues 98-1906 of SEQ ID NO: 1 or from nucleotide residues 11-1486 of SEQ ID NO: 11), as well as 5′-non-translated sequences (nucleotide residues 1-97 of SEQ ID NO: 1 or nucleotide residues 1-10 of SEQ ID NO: 11) or 3′-non-translated sequences (nucleotides 1907-3985 of SEQ ID NO: 1 or nucleotide residues 1487-2590 of SEQ ID NO: 11). Alternatively, the nucleic acid molecule can include only the coding region of one of SEQ ID NOs: 1 and 11 (e.g., nucleotide residues 98-1906, corresponding to SEQ ID NO: 3, or nucleotide residues 11-1486, corresponding to SEQ ID NO: 13) and, e.g., no flanking sequences which normally accompany the subject sequence. In another embodiment, the nucleic acid molecule encodes a sequence corresponding to the 603 amino acid residue protein of SEQ ID NO: 2 or to the 492 amino acid residue protein of SEQ ID NO: 12.

In another embodiment, an isolated nucleic acid molecule of the invention includes a nucleic acid molecule which is a complement of the nucleotide sequence shown in one of SEQ ID NOs: 1, 3, 11, and 13, and a portion of any of these sequences. In other embodiments, the nucleic acid molecule of the invention is sufficiently complementary to the nucleotide sequence shown in one of SEQ ID NOs: 1, 3, 11, and 13 that it can hybridize with a nucleic acid having that sequence, thereby forming a stable duplex.

In one embodiment, an isolated nucleic acid molecule of the invention includes a nucleotide sequence which is at least about 60%, 65%, 70%, 75%, 80%, 85%, 90%, 91%, 92%, 93%, 94%, 95%, 96%, 97%, 98%, or 99% or more homologous to the entire length of the nucleotide sequence shown in one of SEQ ID NOs: 1, 3, 11, and 13, and a portion, preferably of the same length, of any of these nucleotide sequences.

47169/33935 Nucleic Acid Fragments

A nucleic acid molecule of the invention can include only a portion of the nucleic acid sequence of either of SEQ ID NOs: 1 and 3. For example, such a nucleic acid molecule can include a fragment that can be used as a probe or primer or a fragment encoding a portion of a 47169/33935 protein, e.g., an immunogenic or biologically active portion of a 47169/33935 protein. A fragment can comprise nucleotides corresponding to one of residues 148-333 of SEQ ID NO: 2 and residues 286-470 of SEQ ID NO: 12, which encodes a glycosyl transferase domain of human 47169/33935 or to residues 455-590 of SEQ ID NO: 2, which encodes a ricin lectin domain of human 47169. The nucleotide sequence determined from the cloning of the 47169/33935 gene facilitates generation of probes and primers for use in identifying and/or cloning other 47169/33935 family members, or fragments thereof, as well as 47169/33935 homologues, or fragments thereof, from other species.

In another embodiment, a nucleic acid includes a nucleotide sequence that includes part, or all, of the coding region and extends into either (or both) the 5′- or 3′-non-coding region. Other embodiments include a fragment that includes a nucleotide sequence encoding an amino acid fragment described herein. Nucleic acid fragments can encode a specific domain or site described herein or fragments thereof, particularly fragments thereof that are at least about 250 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein.

47169/33935 probes and primers are provided. Typically a probe/primer is an isolated or purified oligonucleotide. The oligonucleotide typically includes a region of nucleotide sequence that hybridizes under stringent conditions to at least about 7, 12 or 15, preferably about 20 or 25, more preferably about 30, 35, 40, 45, 50, 55, 60, 65, or 75 consecutive nucleotides of a sense or antisense sequence of one of SEQ ID NOs: 1, 3, 11, and 13, and a naturally occurring allelic variant or mutant of one of SEQ ID NOs: 1, 3, 11, and 13.

In a preferred embodiment the nucleic acid is a probe which is at least 5 or 10, and less than 200, more preferably less than 100, or less than 50, base pairs in length. It should be identical, or differ by 1, or fewer than 5 or 10 bases, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid that encodes a glycosyl transferase domain at about amino acid residues 148 to 333 of SEQ ID NO: 2 or at about amino acid residues 286 to 470 of SEQ ID NO: 12, a ricin lectin domain at about amino acid residues 455 to 590 of SEQ ID NO: 2, or a predicted transmembrane domain of one of SEQ ID NOs: 2 and 12.

In another embodiment a set of primers is provided, e.g., primers suitable for use in a PCR, which can be used to amplify a selected region of a 47169/33935 sequence. The primers should be at least 5, 10, or 50 base pairs in length and less than 100, or less than 200, base pairs in length. The primers should be identical, or differs by one base from a sequence disclosed herein or from a naturally occurring variant. Primers suitable for amplifying all or a portion of any of the following regions are provided: e.g., one or more a glycosyl transferase domain, a ricin lectin domain, and the predicted transmembrane domain, as defined above relative to SEQ ID NOs: 2 and 12.

A nucleic acid fragment can encode an epitope bearing region of a polypeptide described herein.

A nucleic acid fragment encoding a “biologically active portion of a 47169/33935 polypeptide” can be prepared by isolating a portion of the nucleotide sequence of one of SEQ ID NOs: 1, 3, 11, and 13, which encodes a polypeptide having a 47169/33935 biological activity (e.g., the biological activities of the 47169/33935 proteins are described herein), expressing the encoded portion of the 47169/33935 protein (e.g., by recombinant expression in vitro) and assessing the activity of the encoded portion of the 47169/33935 protein. For example, a nucleic acid fragment encoding a biologically active portion of 47169/33935 includes a glycosyl transferase domain, e.g., amino acid residues 148 to 333 of SEQ ID NO: 2 or amino acid residues 286 to 470 of SEQ ID NO: 12, and a ricin lectin domain, e.g., amino acid residues 455 to 590 of SEQ ID NO: 2. A nucleic acid fragment encoding a biologically active portion of a 47169/33935 polypeptide can comprise a nucleotide sequence that is greater than 25 or more nucleotides in length.

In one embodiment, a nucleic acid includes one that has a nucleotide sequence which is greater than 260, 300, 400, 500, 600, 700, 800, 900, 1000, 1100, 1200, 1300, 1400, 1500, 2000, 2500, 3000, or 3500 or more nucleotides in length and that hybridizes under stringent hybridization conditions with a nucleic acid molecule having the sequence of one of SEQ ID NOs: 1, 3, 11, and 13.

47169/33935 Nucleic Acid Variants

The invention further encompasses nucleic acid molecules having a sequence that differs from the nucleotide sequence shown in one of SEQ ID NOs: 1, 3, 11, and 13. Such differences can be attributable to degeneracy of the genetic code (i.e., differences which result in a nucleic acid that encodes the same 47169/33935 proteins as those encoded by the nucleotide sequence disclosed herein). In another embodiment, an isolated nucleic acid molecule of the invention encodes a protein having an amino acid sequence which differs by at least 1, but by fewer than 5, 10, 20, 50, or 100, amino acid residues from one of SEQ ID NOs: 2 and 12. If alignment is needed for this comparison the sequences should be aligned for maximum homology. “Looped” out sequences from deletions or insertions, or mismatches, are considered differences.

Nucleic acids of the inventor can be chosen for having codons, which are preferred, or non-preferred, for a particular expression system. For example, the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid has a sequence that differs from that of one of SEQ ID NOs: 1, 3, 11, and 13, e.g., as follows: by at least one, but by fewer than 10, 20, 30, or 40, nucleotide residues; or by at least one but by fewer than 1%, 5%, 10% or 20% of the nucleotide residues in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the nucleotide sequence shown in one of SEQ ID NOs: 1, 3, 11, and 13, or a fragment of one of these sequences. Such nucleic acid molecules can readily be identified as being able to hybridize under stringent conditions, to the nucleotide sequence shown in one of SEQ ID NOs: 1, 3, 11, and 13, or a fragment of one of these sequences. Nucleic acid molecules corresponding to orthologs, homologs, and allelic variants of the 47169/33935 cDNAs of the invention can further be isolated by mapping to the same chromosome or locus as the 47169/33935 gene.

Preferred variants include those that are correlated with any of the 47169/33935 biological activities described herein, e.g., catalyzing formation of a covalent bond between an amino acid residue of a protein (e.g., a serine or threonine residue) and a phosphate moiety.

Allelic variants of 47169/33935 (e.g., human 47169/33935) include both functional and non-functional proteins. Functional allelic variants are naturally occurring amino acid sequence variants of the 47169/33935 protein within a population that maintain the ability to mediate any of the 47169/33935 biological activities described herein.

Functional allelic variants will typically contain only conservative substitution of one or more amino acids of one of SEQ ID NOs: 2 and 12, or substitution, deletion or insertion of non-critical residues in non-critical regions of the protein. Non-functional allelic variants are naturally-occurring amino acid sequence variants of the 47169/33935 (e.g., human 47169/33935) protein within a population that do not have the ability to mediate any of the 47169/33935 biological activities described herein. Non-functional allelic variants will typically contain a non-conservative substitution, a deletion, or insertion, or premature truncation of the amino acid sequence of one of SEQ ID NOs: 2 and 12, or a substitution, insertion, or deletion in critical residues or critical regions of the protein.

Moreover, nucleic acid molecules encoding other 47169/33935 family members and, thus, which have a nucleotide sequence which differs from the 47169/33935 sequences of one of SEQ ID NOs: 1, 3, 11, and 13 are within the scope of the invention.

Antisense Nucleic Acid Molecules, Ribozymes and Modified 47169/33935 Nucleic Acid Molecules

In another aspect, the invention features, an isolated nucleic acid molecule that is antisense to 47169/33935. An “antisense” nucleic acid can include a nucleotide sequence that is complementary to a “sense” nucleic acid encoding a protein, e.g., complementary to the coding strand of a double-stranded cDNA molecule or complementary to an mRNA sequence. The antisense nucleic acid can be complementary to an entire 47169/33935 coding strand, or to only a portion thereof (e.g., the coding region of human 47169/33935 corresponding to one of SEQ ID NOs: 3 and 13). In another embodiment, the antisense nucleic acid molecule is antisense to a “non-coding region” of the coding strand of a nucleotide sequence encoding 47169/33935 (e.g., the 5′- and 3′-non-translated regions).

An antisense nucleic acid can be designed such that it is complementary to the entire coding region of 47169/33935 mRNA, but more preferably is an oligonucleotide that is antisense to only a portion of the coding or non-coding region of 47169/33935 mRNA. For example, the antisense oligonucleotide can be complementary to the region surrounding the translation start site of 47169/33935 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, or 80 or more nucleotide residues in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been sub-cloned in an antisense orientation (i.e., RNA transcribed from the inserted nucleic acid will be of an antisense orientation to a target nucleic acid of interest, described fuirther in the following subsection).

The antisense nucleic acid molecules of the invention are typically administered to a subject (e.g., by direct injection at a tissue site), or generated in situ such that they hybridize with or bind to cellular mRNA and/or genomic DNA encoding a 47169/33935 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an alpha-anomeric nucleic acid molecule. An alpha-anomeric nucleic acid molecule forms specific double-stranded hybrids with complementary RNA in which, contrary to the usual beta-units, the strands run parallel to each other (Gaultier et al., 1987, Nucl. Acids. Res. 15:6625-6641). The antisense nucleic acid molecule can also comprise a 2′-o-methylribonucleotide (Inoue et al., 1987, Nucl. Acids Res. 15:6131-6148) or a chimeric RNA-DNA analogue (Inoue et al., 1987, FEBS Lett. 215:327-330).

In still another embodiment, an antisense nucleic acid of the invention is a ribozyme. A ribozyme having specificity for a 47169/33935-encoding nucleic acid can include one or more sequences complementary to the nucleotide sequence of a 47169/33935 cDNA disclosed herein (i.e., one of SEQ ID NOs: 1, 3, 11, and 13), and a sequence having known catalytic sequence responsible for mRNA cleavage (see, for example, U.S. Pat. No. 5,093,246 or Haselhoff et al. (1988, Nature 334:585-591). For example, a derivative of a Tetrahymena L-19 IVS RNA can be constructed in which the nucleotide sequence of the active site is complementary to the nucleotide sequence to be cleaved in a 47169/33935-encoding mRNA (e.g., U.S. Pat. No. 4,987,071; and U.S. Pat. No. 5,116,742). Alternatively, 47169/33935 mRNA can be used to select a catalytic RNA having a specific ribonuclease activity from a pool of RNA molecules (e.g., Bartel et al., 1993, Science 261:1411-1418).

47169/33935 gene expression can be inhibited by targeting nucleotide sequences complementary to the regulatory region of the 47169/33935 (e.g., the 47169/33935 promoter and/or enhancers) to form triple helical structures that prevent transcription of the 47169/33935 gene in target cells (Helene, 1991, Anticancer Drug Des. 6:569-584; Helene, et al., 1992, Ann. N.Y. Acad. Sci. 660:27-36; Maher, 1992, Bioassays 14:807-815). The potential sequences that can be targeted for triple helix formation can be increased by creating a so-called “switchback” nucleic acid molecule. Switchback molecules are synthesized in an alternating 5′ to 3′, 3′ to 5′ manner, such that they hybridize with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

A 47169/33935 nucleic acid molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (Hyrup et al., 1996, Bioorg. Med. Chem. 4:5-23). As used herein, the terms “peptide nucleic acid” (PNA) refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup et al. (1996, supra; Perry-O'Keefe et al., Proc. Natl. Acad. Sci. USA 93:14670-14675).

PNAs of 47169/33935 nucleic acid molecules can be used in therapeutic and diagnostic applications. For example, PNAs can be used as antisense or anti-gene agents for sequence-specific modulation of gene expression by, for example, inducing transcription or translation arrest or inhibiting replication. PNAs of 47169/33935 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases, as described in Hyrup et al., 1996, supra); or as probes or primers for DNA sequencing or hybridization (Hyrup et al., 1996, supra; Perry-O'Keefe, supra).

In other embodiments, the oligonucleotide can include other appended groups such as peptides (e.g., for targeting host cell receptors in vivo), or agents facilitating transport across the cell membrane (e.g., Letsinger et al., 1989, Proc. Natl. Acad. Sci. USA 86:6553-6556; Lemaitre et al., 1987, Proc. Natl. Acad. Sci. USA 84:648-652; PCT publication number WO 88/09810) or the blood-brain barrier (see, e.g., PCT publication number WO 89/10134). In addition, oligonucleotides can be modified with hybridization-triggered cleavage agents (e.g., Krol et al., 1988, Bio-Techniques 6:958-976) or intercalating agents (e.g., Zon, 1988, Pharm. Res. 5:539-549). To this end, the oligonucleotide can be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a 47169/33935 nucleic acid of the invention, two complementary regions, one having a fluorophore and the other having a quencher, such that the molecular beacon is useful for quantitating the presence of the 47169/33935 nucleic acid of the invention in a sample. Molecular beacon nucleic acids are described, for example, in U.S. Pat. No. 5,854,033, U.S. Pat. No. 5,866,336, and U.S. Pat. No. 5,876,930.

Isolated 47169/33935 Polypeptides

In another aspect, the invention features, an isolated 47169/33935 protein, or fragment, e.g., a biologically active portion, for use as immunogens or antigens to raise or test (or more generally to bind) anti-47169/33935 antibodies. 47169/33935 protein can be isolated from cells or tissue sources using standard protein purification techniques. 47169/33935 protein or fragments thereof can be produced by recombinant DNA techniques or synthesized chemically.

Polypeptides of the invention include those that arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when the polypeptide is expressed in a native cell, or in systems which result in the alteration or omission of post-translational modifications, e.g., glycosylation or cleavage, present when expressed in a native cell.

In a preferred embodiment, a 47169/33935 polypeptide has one or more of the following characteristics:
(1) it catalyzes formation of a covalent bond between a saccharide moiety and a hydroxyl or amino moiety of a protein or lipid;
(2) it facilitates non-covalent binding between a protein and a cell, virus, or other protein;
(3) it modulates cell signaling;
(4) it modulates cell differentiation;
(5) it modulates tumorigenesis;
(6) it modulates cell adhesion;
(7) it modulates cell motility;
(8) it modulates a cell-to-cell interaction;
(9) it modulates cell invasivity (e.g., metastatic or extravasative capacity);
(10) it modulates gene transcription; and
(11) it modulates an immune response (e.g., an autoimmune response or a response to a pathogen);
(12) it has a molecular weight, amino acid composition or other physical characteristic of a 47169 protein of SEQ ID NO: 2;
(13) it has an overall sequence similarity (identity) of at least 60-65%, preferably at least 70%, more preferably at least 75, 80, 85, 86, 87, 88, 89, 90, 91, 92, 93, 94, 95, 96, 97, 98, or 99% or more, with a portion of SEQ ID NO: 2;
(14) it has a transmembrane domain which is preferably about 70%, 80%, 90%, 95%, 96%, 97%, 98%, or 99% or more, identical with amino acid residues 8 to 31 of SEQ ID NO: 2;
(15) it has at least one non-transmembrane domain which is preferably about 70%, 80%, 90%, 95%, 96%, 97%, 98%, or 99% or more, identical with amino acid residues 32 to 603 of SEQ ID NO: 2;
(16) it has at least one glycosyl transferase domain which is preferably about 70%, 80%, 90%, 95%, 96%, 97%, 98%, or 99% or more, identical with amino acid residues 148 to 333 of SEQ ID NO: 2;
(17) it has a ricin lectin domain which is preferably about 70%, 80%, 90%, 95%, 96%, 97%, 98%, 99% or higher, identical with amino acid residues 455 to 590 of SEQ ID NO: 2;
(18) it has a molecular weight, amino acid composition or other physical characteristic of a 33935 protein of SEQ ID NO: 12;
(19) it has an overall sequence similarity (identity) of at least 60-65%, preferably at least 70%, more preferably at least 75, 80, 85, 86, 87, 88, 89, 90, 91, 92, 93, 94, 95, 96, 97, 98, or 99% or more, with a portion of SEQ ID NO: 12;
(20) it has a transmembrane domain which is preferably about 70%, 80%, 90%, 95%, 96%, 97%, 98%, or 99% or more, identical with a region selected from the group consisting of the regions corresponding to amino acid residues 16-40, 143-161, 179-197, 234-256, and 400-416 of SEQ ID NO: 12; or
(21) it has at least one glycosyl transferase domain which is preferably about 70%, 80%, 90%, 95%, 96%, 97%, 98%, or 99% or more, identical with amino acid residues 286 to 470 of SEQ ID NO: 12.

In a preferred embodiment, the 47169/33935 protein or fragment thereof differs only insubstantially, if at all, from the corresponding sequence in one of SEQ ID NOs: 2 and 12. In one embodiment, it differs by at least one, but by fewer than 15, 10 or 5 amino acid residues. In another, it differs from the corresponding sequence in one of SEQ ID NOs: 2 and 12 by at least one residue but fewer than 20%, 15%, 10% or 5% of the residues differ from the corresponding sequence in one of SEQ ID NOs: 2 and 12 (if this comparison requires alignment the sequences should be aligned for maximum homology. “Looped” out sequences from deletions or insertions, or mismatches, are considered differences). The differences are, preferably, differences or changes at a non-essential amino acid residues or involve a conservative substitution of one residue for another. In a preferred embodiment the differences are not in residues 148-333 or 455-590 of SEQ ID NO: 2 or in residues 286 to 470 of SEQ ID NO: 12.

Other embodiments include a protein that has one or more changes in amino acid sequence, relative to one of SEQ ID NOs: 2 and 12 (e.g., a change in an amino acid residue which is not essential for activity). Such 47169/33935 proteins differ in amino acid sequence from one of SEQ ID NOs: 2 and 12, yet retain biological activity.

In one embodiment, the protein includes an amino acid sequence at least about 60%, 65%, 70%, 75%, 80%, 85%, 90%, 95%, 98% or more homologous to one of SEQ ID NOs: 2 and 12.

A 47169/33935 protein or fragment is provided which has an amino acid sequence which varies from SEQ ID NO: 2 in one or more of the regions corresponding to residues 1-147, 334-454, or 590-603 of SEQ ID NO: 2 or 1-485 or 471-492 of SEQ ID NO: 12 by at least one, but by fewer than 15, 10 or 5 amino acid residues, but which does not differ from SEQ ID NO: 2 in the region corresponding to residues 148-333 or in the region corresponding to residues 455-590 of SEQ ID NO: 2 or from SEQ ID NO: 12 in the region corresponding to residues 286-470 of SEQ ID NO: 12 (if this comparison requires alignment the sequences should be aligned for maximum homology. “Looped” out sequences from deletions or insertions, or mismatches, are considered differences). In some embodiments the difference is at a non-essential residue or is a conservative substitution, while in others the difference is at an essential residue or is a non-conservative substitution.

A biologically active portion of a 47169/33935 protein should include at least the 47169/33935 glycosyl transferase domain, and it should also include the 47169/33935 ricin lectin domain. Moreover, other biologically active portions, in which other regions of the protein are deleted, can be prepared by recombinant techniques and evaluated for one or more of the functional activities of a native 47169/33935 protein.

In a preferred embodiment, the 47169/33935 protein has the amino acid sequence one of SEQ ID NOs: 2 and 12. In other embodiments, the 47169/33935 protein is substantially identical to one of SEQ ID NOs: 2 and 12. In yet another embodiment, the 47169/33935 protein is substantially identical to one of SEQ ID NOs: 2 and 12 and retains the functional activity of the protein of one of SEQ ID NOs: 2 and 12.

47169/33935 Chimeric or Fusion Proteins

In another aspect, the invention provides 47169/33935 chimeric or fusion proteins. As used herein, a 47169/33935 “chimeric protein” or “fusion protein” includes a 47169/33935 polypeptide linked to a non-47169/33935 polypeptide. A “non-47169/33935 polypeptide” refers to a polypeptide having an amino acid sequence corresponding to a protein which is not substantially homologous to the 47169/33935 protein, e.g., a protein which is different from the 47169/33935 protein and which is derived from the same or a different organism. The 47169/33935 polypeptide of the fusion protein can correspond to all or a portion e.g., a fragment described herein of a 47169/33935 amino acid sequence. In a preferred embodiment, a 47169/33935 fusion protein includes at least one or more biologically active portions of a 47169/33935 protein. The non-47169/33935 polypeptide can be fused to the amino or carboxyl terminus of the 47169/33935 polypeptide.

The fusion protein can include a moiety that has a high affinity for a ligand. For example, the fusion protein can be a GST-47169/33935 fusion protein in which the 47169/33935 sequences are fused to the carboxyl terminus of the GST sequences. Such fusion proteins can facilitate purification of recombinant 47169/33935. Alternatively, the fusion protein can be a 47169/33935 protein containing a heterologous signal sequence at its amino terminus. In certain host cells (e.g., mammalian host cells), expression and/or secretion of 47169/33935 can be increased through use of a heterologous signal sequence.

Fusion proteins can include all or a part of a serum protein, e.g., an IgG constant region, or human serum albumin.

The 47169/33935 fusion proteins of the invention can be incorporated into pharmaceutical compositions and administered to a subject in vivo. The 47169/33935 fusion proteins can be used to affect the bioavailability of a 47169/33935 substrate. 47169/33935 fusion proteins can be useful therapeutically for the treatment of disorders caused by, for example, (i) aberrant modification or mutation of a gene encoding a 47169/33935 protein; (ii) mis-regulation of the 47169/33935 gene; and (iii) aberrant post-translational modification of a 47169/33935 protein.

Moreover, the 47169/33935-fusion proteins of the invention can be used as immunogens to produce anti-47169/33935 antibodies in a subject, to purify 47169/33935 ligands and in screening assays to identify molecules that inhibit the interaction of 47169/33935 with a 47169/33935 substrate.

Expression vectors are commercially available that already encode a fusion moiety (e.g., a GST polypeptide). A 47169/33935-encoding nucleic acid can be cloned into such an expression vector such that the fusion moiety is linked in-frame to the 47169/33935 protein.

Variants of 47169/33935 Proteins

In another aspect, the invention also features a variant of a 47169/33935 polypeptide, e.g., which functions as an agonist (mimetics) or as an antagonist. Variants of the 47169/33935 proteins can be generated by mutagenesis, e.g., discrete point mutation, the insertion or deletion of sequences or the truncation of a 47169/33935 protein. An agonist of the 47169/33935 proteins can retain substantially the same, or a subset, of the biological activities of the naturally occurring form of a 47169/33935 protein. An antagonist of a 47169/33935 protein can inhibit one or more of the activities of the naturally occurring form of the 47169/33935 protein by, for example, competitively modulating a 47169/33935-mediated activity of a 47169/33935 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the 47169/33935 protein.

Variants of a 47169/33935 protein can be identified by screening combinatorial libraries of mutants, e.g., truncation mutants, of a 47169/33935 protein for agonist or antagonist activity.

Libraries of fragments e.g., amino-terminal, carboxyl-terminal, or internal fragments, of a 47169/33935 protein coding sequence can be used to generate a variegated population of fragments for screening and subsequent selection of variants of a 47169/33935 protein.

Variants in which a cysteine residue is added or deleted or in which a residue that is glycosylated is added or deleted are particularly preferred.

Methods for screening gene products of combinatorial libraries made by point mutations or truncation, and for-screening cDNA libraries for gene products having a selected property. Recursive ensemble mutagenesis (REM), a technique which enhances the frequency of functional mutants in the libraries, can be used in combination with the screening assays to identify 47169/33935 variants (Arkin et al., 1992, Proc. Natl. Acad. Sci. USA 89:7811-7815; Delgrave et al., 1993, Protein Engr. 6:327-331).

Cell based assays can be exploited to analyze a variegated 47169/33935 library. For example, a library of expression vectors can be transfected into a cell line, e.g., a cell line, which ordinarily responds to 47169/33935 in a substrate-dependent manner. The transfected cells are then contacted with 47169/33935 and the effect of the expression of the mutant on signaling by the 47169/33935 substrate can be detected, e.g., by measuring changes in cell growth and/or enzymatic activity. Plasmid DNA can then be recovered from the cells that score for inhibition, or alternatively, potentiation of signaling by the 47169/33935 substrate, and the individual clones further characterized.

In another aspect, the invention features a method of making a 47169/33935 polypeptide, e.g., a peptide having a non-wild-type activity, e.g., an antagonist, agonist, or super agonist of a naturally-occurring 47169/33935 polypeptide, e.g., a naturally-occurring 47169/33935 polypeptide. The method includes: altering the sequence of a 47169/33935 polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a 47169/33935 polypeptide a biological activity of a naturally occurring 47169/33935 polypeptide. The method includes: altering the sequence, e.g., by substitution or deletion of one or more residues, of a 47169/33935 polypeptide, e.g., altering the sequence of a non-conserved region, or a domain or residue described herein, and testing the altered polypeptide for the desired activity.

Anti-47169/33935 Antibodies

In another aspect, the invention provides an anti-47169/33935 antibody. The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as pepsin.

The antibody can be a polyclonal, monoclonal, recombinant, e.g., a chimeric or humanized, fully-human, non-human, e.g., murine, or single chain antibody. In a preferred embodiment, it has effector function and can fix complement. The antibody can be coupled to a toxin or imaging agent.

A full-length 47169/33935 protein or, antigenic peptide fragment of 47169/33935 can be used as an immunogen or can be used to identify anti-47169/33935 antibodies made with other immunogens, e.g., cells, membrane preparations, and the like. The antigenic peptide of 47169/33935 should include at least 8 amino acid residues of the amino acid sequence shown in one of SEQ ID NOs: 2 and 12 and encompasses an epitope of 47169/33935. Preferably, the antigenic peptide includes at least 10 amino acid residues, more preferably at least 15 amino acid residues, even more preferably at least 20 amino acid residues, and most preferably at least 30 amino acid residues.

Fragments of 47169 which include about residues 220-235 of SEQ ID NO: 2 can be used to make antibodies, e.g., for use as immunogens or to characterize the specificity of an antibody, against hydrophobic regions of the 47169 protein. Similarly, a fragment of 47169 which include about residues 65-110 or 400-415 of SEQ ID NO: 2 can be used to make an antibody against a hydrophilic region of the 47169 protein.

Fragments of 33935 which include about residues 16-40 of SEQ ID NO: 12 can be used to make antibodies, e.g., for use as immunogens or to characterize the specificity of an antibody, against hydrophobic regions of the 33935 protein. Similarly, a fragment of 33935 which include about residues 340-360 of SEQ ID NO: 12 can be used to make an antibody against a hydrophilic region of the 33935 protein.

Antibodies reactive with, or specific for, any of these regions, or other regions or domains described herein are provided.

Preferred epitopes encompassed by the antigenic peptide are regions of 47169/33935 are located on the surface of the protein, e.g., hydrophilic regions, as well as regions with high antigenicity. For example, an Emini surface probability analysis of the human 47169/33935 protein sequence can be used to indicate the regions that have a particularly high probability of being localized to the surface of the 47169/33935 protein and are thus likely to constitute surface residues useful for targeting antibody production.

In a preferred embodiment the antibody binds an epitope on any domain or region on 47169/33935 proteins described herein.

Chimeric, humanized, but most preferably, completely human antibodies are desirable for applications which include repeated administration, e.g., therapeutic treatment (and some diagnostic applications) of human patients.

The anti-47169/33935 antibody can be a single chain antibody. A single-chain antibody (scFV) can be engineered (e.g., Colcher et al., 1999, Ann. N.Y. Acad. Sci. 880:263-280; Reiter, 1996, Clin. Cancer Res. 2:245-252). The single chain antibody can be dimerized or multimerized to generate multivalent antibodies having specificities for different epitopes of the same target 47169/33935 protein.

In a preferred embodiment, the antibody has reduced or no ability to bind an Fc receptor. For example, it can be an isotype, subtype, fragment or other mutant, which does not support binding to an Fc receptor, e.g., it can have a mutated or deleted Fc receptor binding region.

An anti-47169/33935 antibody (e.g., monoclonal antibody) can be used to isolate 47169/33935 by standard techniques, such as affinity chromatography or immunoprecipitation. Moreover, an anti-47169/33935 antibody can be used to detect 47169/33935 protein (e.g., in a cellular lysate or cell supernatant) in order to evaluate the abundance and pattern of expression of the protein. Anti-47169/33935 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a 47169/33935 nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., 47169/33935 proteins, mutant forms of 47169/33935 proteins, fusion proteins, and the like).

The recombinant expression vectors of the invention can be designed for expression of 47169/33935 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel (1990, Gene Expression Technology: Methods in Enzymology 185, Academic Press, San Diego). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith et al., 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in 47169/33935 activity assays, (e.g., direct assays or competitive assays described in detail below), or to generate antibodies specific for 47169/33935 proteins. In a preferred embodiment, a fusion protein expressed in a retroviral expression vector of the present invention can be used to infect bone marrow cells that are subsequently transplanted into irradiated recipients. The pathology of the subject recipient is then examined after sufficient time has passed (e.g., six weeks).

To maximize recombinant protein expression in E. coli, the protein is expressed in a host bacterial strain with an impaired capacity to proteolytically cleave the recombinant protein (Gottesman, 1990, Gene Expression Technology: Methods in Enzymology 185, Academic Press, San Diego, 119-128). Another strategy is to alter the nucleic acid sequence of the nucleic acid to be inserted into an expression vector so that the individual codons for each amino acid are those preferentially utilized in E. coli (Wada et al., 1992, Nucl. Acids Res. 20:2111-2118). Such alteration of nucleic acid sequences of the invention can be carried out by standard DNA synthesis techniques.

The 47169/33935 expression vector can be a yeast expression vector, a vector for expression in insect cells, e.g., a baculovirus expression vector, or a vector suitable for expression in mammalian cells.

When used in mammalian cells, the expression vector's control functions are often provided by viral regulatory elements. For example, commonly used viral promoters are derived from polyoma, adenovirus 2, cytomegalovirus and simian virus 40 (SV40).

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al., 1987, Genes Dev. 1:268-277), lymphoid-specific promoters (Calame et al., 1988, Adv. Immunol. 43:235-275), in particular promoters of T cell receptors (Winoto et al., 1989, EMBO J. 8:729-733) and immunoglobulins (Banerji et al., 1983, Cell 33:729-740; Queen et al., 1983, Cell 33:741-748), neuron-specific promoters (e.g., the neurofilament promoter; Byrne et al., 1989, Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Patent Application publication number 264,166). Developmentally-regulated promoters are also encompassed, for example, the murine hox promoters (Kessel et al., 1990, Science 249:374-379) and the alpha-fetoprotein promoter (Campes et al., 1989, Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes, see Weintraub, H. et al. (1986, Trends Genet. 1:Review).

Another aspect the invention provides a host cell which includes a nucleic acid molecule described herein, e.g., a 47169/33935 nucleic acid molecule within a recombinant expression vector or a 47169/33935 nucleic acid molecule containing sequences which allow it to homologously recombine into a specific site of the host cell's genome. The terms “host cell” and “recombinant host cell” are used interchangeably herein. Such terms refer not only to the particular subject cell, but also to the progeny or potential progeny of such a cell. Because certain modifications can occur in succeeding generations due to either mutation or environmental influences, such progeny may not, in fact, be identical to the parent cell, but are included within the scope of the term as used herein.

A host cell can be any prokaryotic or eukaryotic cell. For example, a 47169/33935 protein can be expressed in bacterial cells such as E. coli, insect cells, yeast or mammalian cells (such as Chinese hamster ovary (CHO) cells) or COS cells. Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a 47169/33935 protein. Accordingly, the invention further provides methods for producing a 47169/33935 protein using the host cells of the invention. In one embodiment, the method includes culturing the host cell of the invention (into which a recombinant expression vector encoding a 47169/33935 protein has been introduced) in a suitable medium such that a 47169/33935 protein is produced. In another embodiment, the method further includes isolating a 47169/33935 protein from the medium or the host cell.

In another aspect, the invention features, a cell or purified preparation of cells which include a 47169/33935 transgene, or which otherwise mal-express 47169/33935. The cell preparation can consist of human or non-human cells, e.g., rodent cells, e.g., mouse or rat cells, rabbit cells, or pig cells. In preferred embodiments, the cell or cells include a 47169/33935 transgene, e.g., a heterologous form of a 47169/33935, e.g., a gene derived from humans (in the case of a non-human cell). The 47169/33935 transgene can be mal-expressed, e.g., over-expressed or under-expressed. In other preferred embodiments, the cell or cells include a gene that mal-expresses an endogenous 47169/33935, e.g., a gene the expression of which is disrupted, e.g., a knockout. Such cells can serve as a model for studying disorders that are related to mutated or mal-expressed 47169/33935 alleles or for use in drug screening.

In another aspect, the invention includes, a human cell, e.g., a hematopoietic stem cell, transformed with nucleic acid that encodes a subject 47169/33935 polypeptide.

Also provided are cells, preferably human cells, e.g., human hematopoietic or fibroblast cells, in which an endogenous 47169/33935 is under the control of a regulatory sequence that does not normally control expression of the endogenous 47169/33935 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 47169/33935 gene. For example, an endogenous 47169/33935 gene that is “transcriptionally silent,” e.g., not normally expressed, or expressed only at very low levels, can be activated by inserting a regulatory element that is capable of promoting the expression of a normally expressed gene product in that cell. Techniques such as targeted homologous recombination, can be used to insert the heterologous DNA as described (e.g., U.S. Pat. No. 5,272,071; PCT publication number WO 91/06667).

Transgenic Animals

The invention provides non-human transgenic animals. Such animals are useful for studying the function and/or activity of a 47169/33935 protein and for identifying and/or evaluating modulators of 47169/33935 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous 47169/33935 gene has been altered, e.g., by homologous recombination between the endogenous gene and an exogenous DNA molecule introduced into a cell of the animal (e.g., an embryonic cell of the animal, prior to development of the animal).

Intronic sequences and polyadenylation signals can also be included in the transgene to increase the efficiency of expression of the transgene. A tissue-specific regulatory sequence(s) can be operably linked to a transgene of the invention to direct expression of a 47169/33935 protein to particular cells. A transgenic founder animal can be identified based upon the presence of a 47169/33935 transgene in its genome and/or expression of 47169/33935 mRNA in tissues or cells of the animals. A transgenic founder animal can then be used to breed additional animals carrying the transgene. Moreover, transgenic animals carrying a transgene encoding a 47169/33935 protein can further be bred to other transgenic animals carrying other transgenes.

47169/33935 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk- or egg-specific promoter, and recovered from the milk or eggs produced by the animal. Suitable animals are mice, pigs, cows, goats, and sheep.

The invention also includes a population of cells from a transgenic animal, as discussed, e.g., below.

Uses

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). The isolated nucleic acid molecules of the invention can be used, for example, to express a 47169/33935 protein (e.g., via a recombinant expression vector in a host cell in gene therapy applications), to detect a 47169/33935 mRNA (e.g., in a biological sample), to detect a genetic alteration in a 47169/33935 gene and to modulate 47169/33935 activity, as described further below. The 47169/33935 proteins can be used to treat disorders characterized by insufficient or excessive production of a 47169/33935 substrate or production of 47169/33935 inhibitors. In addition, the 47169/33935 proteins can be used to screen for naturally occurring 47169/33935 substrates, to screen for drugs or compounds which modulate 47169/33935 activity, as well as to treat disorders characterized by insufficient or excessive production of 47169/33935 protein or production of 47169/33935 protein forms which have decreased, aberrant or unwanted activity compared to 47169/33935 wild-type protein. Examples of such disorders include those in which protein or lipid glycosylation is aberrant. Examples of such disorders include diabetes mellitus, hypoglycemia, arthritis, rheumatism, autoimmune disorders (e.g., systemic lupus erythematosus, Graves' disease, myasthenia gravis, insulin resistance, rheumatoid arthritis, scleroderma, and autoimmune infertility), tumorigenesis, and tumor metastasis. Moreover, the anti-47169/33935 antibodies of the invention can be used to detect and isolate 47169/33935 proteins, regulate the bioavailability of 47169/33935 proteins, and modulate 47169/33935 activity.

A method of evaluating a compound for the ability to interact with, e.g., bind to, a subject 47169/33935 polypeptide is provided. The method includes: contacting the compound with the subject 47169/33935 polypeptide; and evaluating the ability of the compound to interact with, e.g., to bind or form a complex with, the subject 47169/33935 polypeptide. This method can be performed in vitro, e.g., in a cell free system, or in vivo, e.g., in a two-hybrid interaction trap assay. This method can be used to identify naturally-occurring molecules that interact with a subject 47169/33935 polypeptide. It can also be used to find natural or synthetic inhibitors of a subject 47169/33935 polypeptide. Screening methods are discussed in more detail below.

Screening Assays

The invention provides screening methods (also referred to herein as “assays”) for identifying modulators, i.e., candidate or test compounds or agents (e.g., proteins, peptides, peptidomimetics, peptoids, small molecules or other drugs) which bind with 47169/33935 proteins, have a stimulatory or inhibitory effect on, for example, 47169/33935 expression or 47169/33935 activity, or have a stimulatory or inhibitory effect on, for example, the expression or activity of a 47169/33935 substrate. Compounds thus identified can be used to modulate the activity of target gene products (e.g., 47169/33935 genes) in a therapeutic protocol, to elaborate the biological function of the target gene product, or to identify compounds that disrupt normal target gene interactions.

In one embodiment, the invention provides assays for screening candidate or test compounds that are substrates of a 47169/33935 protein or polypeptide or a biologically active portion thereof. In another embodiment, the invention provides assays for screening candidate or test compounds that bind to or modulate the activity of a 47169/33935 protein or polypeptide or a biologically active portion thereof.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; e.g., Zuckermann et al., 1994, J. Med. Chem. 37:2678-2685); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries have been described (e.g., DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233).

Libraries of compounds can be presented in solution (e.g., Houghten, 1992, Biotechniques 13:412-421), or on beads (Lam, 1991, Nature 354:82-84), chips (Fodor, 1993, Nature 364:555-556), bacteria (U.S. Pat. No. 5,223,409), spores (U.S. Pat. No. 5,223,409), plasmids (Cull et al., 1992, Proc. Natl. Acad. Sci. USA 89:1865-1869), or on phage (Scott et al., 1990, Science 249:386-390; Devlin, 1990, Science 249:404-406; Cwirla et al., 1990, Proc. Natl. Acad. Sci. USA 87:6378-6382; Felici, 1991, J. Mol. Biol. 222:301-310; U.S. Pat. No. 5,223,409).

In one embodiment, an assay is a cell-based assay in which a cell which expresses a 47169/33935 protein or biologically active portion thereof is contacted with a test compound, and the ability of the test compound to modulate 47169/33935 activity is determined. Determining the ability of the test compound to modulate 47169/33935 activity can be accomplished by monitoring, for example, changes in enzymatic activity. The cell, for example, can be of mammalian origin.

The ability of the test compound to modulate 47169/33935 binding to a compound, e.g., a 47169/33935 substrate, or to bind to 47169/33935 can also be evaluated. This can be accomplished, for example, by coupling the compound, e.g., the substrate, with a radioisotope or enzymatic label such that binding of the compound, e.g., the substrate, to 47169/33935 can be determined by detecting the labeled compound, e.g., substrate, in a complex. Alternatively, 47169/33935 could be coupled with a radioisotope or enzymatic label to monitor the ability of a test compound to modulate 47169/33935 binding to a 47169/33935 substrate in a complex. For example, compounds (e.g., 47169/33935 substrates) can be labeled with ¹²⁵I, ³⁵S, ¹⁴C, or ³H, either directly or indirectly, and the radioisotope detected by direct counting of radio-emission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a 47169/33935 substrate) to interact with 47169/33935 with or without the labeling of any of the interactants can be evaluated. For example, a microphysiometer can be used to detect the interaction of a compound with 47169/33935 without the labeling of either the compound or the 47169/33935 (McConnell et al., 1992, Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 47169/33935.

In yet another embodiment, a cell-free assay is provided in which a 47169/33935 protein or biologically active portion thereof is contacted with a test compound and the ability of the test compound to bind to the 47169/33935 protein or biologically active portion thereof is evaluated. Preferred biologically active portions of the 47169/33935 proteins to be used in assays of the present invention include fragments that participate in interactions with non-47169/33935 molecules, e.g., fragments with high surface probability scores.

Soluble and/or membrane-bound forms of isolated proteins (e.g., 47169/33935 proteins or biologically active portions thereof) can be used in the cell-free assays of the invention. When membrane-bound forms of the protein are used, it can be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)n, 3-{(3-cholamidopropyl) dimethylamminio}-1-propane sulfonate (CHAPS), 3-{(3-cholamidopropyl) dimethylamminio}-2-hydroxy-1-propane sulfonate (CHAPSO), or N-dodecyl-N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET; e.g., U.S. Pat. No. 5,631,169; U.S. Pat. No. 4,868,103). A fluorophore label is selected such that a first donor molecule's emitted fluorescent energy will be absorbed by a fluorescent label on a second, ‘acceptor’ molecule, which in turn is able to fluoresce due to the absorbed energy. Alternately, the ‘donor’ protein molecule can simply utilize the natural fluorescent energy of tryptophan residues. Labels are chosen that emit different wavelengths of light, such that the ‘acceptor’ molecule label can be differentiated from that of the ‘donor.’ Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the 47169/33935 protein to bind to a target molecule can be accomplished using real-time biomolecular interaction analysis (BIA; e.g., Sjolander et al., 1991, Anal. Chem. 63:2338-2345; Szabo et al., 1995, Curr. Opin. Struct. Biol. 5:699-705). “Surface plasmon resonance” (SPR) or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of SPR), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It can be desirable to immobilize either 47169/33935, an anti-47169/33935 antibody or its target molecule to facilitate separation of complexed from non-complexed forms of one or both of the proteins, as well as to accommodate automation of the assay. Binding of a test compound to a 47169/33935 protein, or interaction of a 47169/33935 protein with a target molecule in the presence and absence of a candidate compound, can be accomplished in any vessel suitable for containing the reactants. Examples of such vessels include microtiter plates, test tubes, and micro-centrifuige tubes. In one embodiment, a fusion protein can be provided which adds a domain that allows one or both of the proteins to be bound to a matrix. For example, glutathione-S-transferase/47169/33935 fusion proteins or glutathione-S-transferase/target fusion proteins can be adsorbed onto glutathione Sepharose™ beads (Sigma Chemical, St. Louis, Mo.) or glutathione-derivatized microtiter plates, which are then combined with the test compound or the test compound and either the non-adsorbed target protein or 47169/33935 protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of 47169/33935 binding or activity determined using standard techniques.

Other techniques for immobilizing either a 47169/33935 protein or a target molecule on matrices include using conjugation of biotin and streptavidin. Biotinylated 47169/33935 protein or target molecules can be prepared from biotin-N-hydroxy-succinimide using techniques known in the art (e.g., biotinylation kit, Pierce Chemicals, Rockford, Ill., and immobilized in the wells of streptavidin-coated 96 well plates (Pierce Chemical).

In order to conduct the assay, the non-immobilized component is added to the coated surface containing the anchored component. After the reaction is complete, non-reacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with 47169/33935 protein or target molecules but which do not interfere with binding of the 47169/33935 protein to its target molecule. Such antibodies can be derivatized to the wells of the plate, and unbound target or 47169/33935 protein trapped in the wells by antibody conjugation. Methods for detecting such complexes, in addition to those described above for the GST-immobilized complexes, include immunodetection of complexes using antibodies reactive with the 47169/33935 protein or target molecule, as well as enzyme-linked assays which rely on detecting an enzymatic activity associated with the 47169/33935 protein or target molecule.

Alternatively, cell free assays can be conducted in a liquid phase. In such an assay, the reaction products are separated from non-reacted components, by any of a number of standard techniques, including, but not limited to: differential centrifugation (e.g., Rivas et al., 1993, Trends Biochem. Sci. 18:284-287); chromatography (e.g., gel filtration chromatography or ion-exchange chromatography); electrophoresis (e.g., Ausubel et al., eds., 1999, Current Protocols in Molecular Biology, J. Wiley, New York); and immunoprecipitation (e.g., Ausubel, supra). Such resins and chromatographic techniques are known to one skilled in the art (e.g., Heegaard, 1998, J. Mol. Recognit. 11:141-148; Hage et al., 1997, J. Chromatogr. B Biomed. Sci. Appl. 699:499-525). Further, fluorescence energy transfer can also be conveniently utilized, as described herein, to detect binding without further purification of the complex from solution.

In a preferred embodiment, the assay includes contacting the 47169/33935 protein or biologically active portion thereof with a known compound which binds 47169/33935 to form an assay mixture, contacting the assay mixture with a test compound, and determining the ability of the test compound to interact with a 47169/33935 protein, wherein determining the ability of the test compound to interact with a 47169/33935 protein includes determining the ability of the test compound to preferentially bind to 47169/33935 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the 47169/33935 genes herein identified. In an alternative embodiment, the invention provides methods for determining the ability of the test compound to modulate the activity of a 47169/33935 protein through modulation of the activity of a downstream effector of a 47169/33935 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, non-reacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from non-reacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a pre-formed complex of the target gene product and the interactive cellular or extracellular binding partner product is prepared in that either the target gene products or their binding partners are labeled, but the signal generated by the label is quenched due to complex formation (e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the 47169/33935 proteins can be used as “bait proteins” in a two-hybrid assay or three-hybrid assay (e.g., U.S. Pat. No. 5,283,317; Zervos et al., 1993, Cell 72:223-232; Madura et al., 1993, J. Biol. Chem. 268:12046-12054; Bartel et al., 1993, Biotechniques 14:920-924; Iwabuchi et al., 1993, Oncogene 8:1693-1696; PCT publication number WO 94/10300), to identify other proteins, which bind to or interact with 47169/33935 (“47169/33935-binding proteins” or “47169/33935-bp”) and are involved in 47169/33935 activity. Such 47169/33935-bps can be activators or inhibitors of signals by the 47169/33935 proteins or 47169/33935 targets as, for example, downstream elements of a 47169/33935-mediated signaling pathway.

The two-hybrid system is based on the modular nature of most transcription factors, which consist of separable DNA-binding and activation domains. Briefly, the assay utilizes two different DNA constructs. In one construct, the gene that codes for a 47169/33935 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively, the 47169/33935 protein can be fused to the activator domain). If the “bait” and the “prey” proteins are able to interact in vivo forming a 47169/33935-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein that interacts with the 47169/33935 protein.

In another embodiment, modulators of 47169/33935 expression are identified. For example, a cell or cell free mixture is contacted with a candidate compound and the expression of 47169/33935 mRNA or protein evaluated relative to the level of expression of 47169/33935 mRNA or protein in the absence of the candidate compound. When expression of 47169/33935 mRNA or protein is greater in the presence of the candidate compound than in its absence, the candidate compound is identified as a stimulator of 47169/33935 mRNA or protein expression. Alternatively, when expression of 47169/33935 mRNA or protein is less (i.e., statistically significantly less) in the presence of the candidate compound than in its absence, the candidate compound is identified as an inhibitor of 47169/33935 mRNA or protein expression. The level of 47169/33935 mRNA or protein expression can be determined by methods described herein for detecting 47169/33935 mRNA or protein.

In another aspect, the invention pertains to a combination of two or more of the assays described herein. For example, a modulating agent can be identified using a cell-based or a cell free assay, and the ability of the agent to modulate the activity of a 47169/33935 protein can be confirmed in vivo, e.g., in an animal such as an animal model for a disease.

This invention further pertains to novel agents identified by the above-described screening assays. Accordingly, it is within the scope of this invention to further use an agent identified as described herein (e.g., a 47169/33935 modulating agent, an antisense 47169/33935 nucleic acid molecule, a 47169/33935-specific antibody, or a 47169/33935-binding partner) in an appropriate animal model to determine the efficacy, toxicity, side effects, or mechanism of action, of treatment with such an agent. Furthermore, novel agents identified by the above-described screening assays can be used for treatments as described herein.

Detection Assays

Portions or fragments of the nucleic acid sequences identified herein can be used as polynucleotide reagents. For example, these sequences can be used to: (i) map their respective genes on a chromosome, e.g., to locate gene regions associated with genetic disease or to associate 47169/33935 with a disease; (ii) identify an individual from a minute biological sample (tissue typing); and (iii) aid in forensic identification of a biological sample. These applications are described in the subsections below.

Chromosome Mapping

The 47169/33935 nucleotide sequences or portions thereof can be used to map the location of the 47169/33935 genes on a chromosome. This process is called chromosome mapping. Chromosome mapping is useful in correlating the 47169/33935 sequences with genes associated with disease.

Briefly, 47169/33935 genes can be mapped to chromosomes by preparing PCR primers (preferably 15-25 base pairs in length) from the 47169/33935 nucleotide sequence (e.g., one of SEQ ID NOs: 1, 3, 11, and 13). These primers can then be used for PCR screening of somatic cell hybrids containing individual human chromosomes. Only those hybrids containing the human gene corresponding to the 47169/33935 sequences will yield an amplified fragment.

A panel of somatic cell hybrids in which each cell line contains either a single human chromosome or a small number of human chromosomes, and a full set of mouse chromosomes, can allow easy mapping of individual genes to specific human chromosomes (D'Eustachio et al., 1983, Science 220:919-924).

Other mapping strategies e.g., in situ hybridization as described (Fan et al., 1990, Proc. Natl. Acad. Sci. USA 87:6223-6227), pre-screening with labeled flow-sorted chromosomes, and pre-selection by hybridization to chromosome specific cDNA libraries can be used to map 47169/33935 to a chromosomal location.

Fluorescence in situ hybridization (FISH) of a DNA sequence to a metaphase chromosomal spread can further be used to provide a precise chromosomal location in one step. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of FISH, see Verma et al. (1988, Human Chromosomes: A Manual of Basic Techniques, Pergamon Press, New York).

Reagents for chromosome mapping can be used individually to mark a single chromosome or a single site on that chromosome, or panels of reagents can be used for marking multiple sites and/or multiple chromosomes. Reagents corresponding to non-coding regions of the genes are typically preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data (such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), as described (e.g., Egeland et al., 1987, Nature, 325:783-787).

Moreover, differences in the DNA sequences between individuals affected and unaffected with a disease associated with the 47169/33935 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

47169/33935 sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the present invention are useful as additional DNA markers for RFLP (described in U.S. Pat. No. 5,272,057).

Furthermore, the sequences of the present invention can also be used to determine the actual base-by-base DNA sequence of selected portions of an individual's genome. Thus, the 47169/33935 nucleotide sequence described herein can be used to prepare PCR primers homologous to the 5′- and 3′-ends of the sequence. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the non-coding regions. Each of the sequences described herein can, to some degree, be used as a standard against which DNA from an individual can be compared for identification purposes. Because greater numbers of polymorphisms occur in the non-coding regions, fewer sequences are necessary to differentiate individuals. The non-coding sequences of one of SEQ ID NOs: 1 and 11 can provide positive individual identification with a panel of perhaps 10 to 1,000 primers which each yield a non-coding amplified sequence of 100 bases. If predicted coding sequences are used, such as those in one of SEQ ID NOs: 3 and 13, a more appropriate number of primers for positive individual identification would be 500-2,000.

If a panel of reagents from 47169/33935 nucleotide sequences described herein is used to generate a unique identification database for an individual, those same reagents can later be used to identify tissue from that individual. Using the unique identification database, positive identification of the individual, living or dead, can be made from extremely small tissue samples.

Use of Partial 47169/33935 Sequences in Forensic Biology

DNA-based identification techniques can also be used in forensic biology. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). As mentioned above, actual nucleotide sequence information can be used for identification as an accurate alternative to patterns formed by restriction enzyme generated fragments. Sequences targeted to non-coding regions of one of SEQ ID NOs: 1 and 11 (e.g., fragments having a length of at least 20 nucleotide residues, preferably at least 30 nucleotide residues) are particularly appropriate for this use.

The 47169/33935 nucleotide sequences described herein can further be used to provide polynucleotide reagents, e.g., labeled or label-able probes which can be used in, for example, an in situ hybridization technique, to identify a specific tissue, e.g., a tissue containing hematopoietic cells. This can be very useful in cases where a forensic pathologist is presented with a tissue of unknown origin. Panels of such 47169/33935 probes can be used to identify tissue by species and/or by organ type.

In a similar fashion, these reagents, e.g., 47169/33935 primers or probes can be used to screen tissue culture for contamination (i.e., to screen for the presence of a mixture of different types of cells in a culture).

Predictive Medicine

The present invention also pertains to the field of predictive medicine in which diagnostic assays, prognostic assays, and monitoring clinical trials are used for prognostic (predictive) purposes to thereby treat an individual.

Generally, the invention provides a method of determining if a subject is at risk for a disorder related to a lesion in, or the malexpression of, a gene that encodes a 47169/33935 polypeptide.

Such disorders include, e.g., a disorder associated with the malexpression of a 47169/33935 polypeptide. Examples of such disorders include diabetes mellitus, hypoglycemia, arthritis, rheumatism, autoimmune disorders (e.g., systemic lupus erythematosus, Graves' disease, myasthenia gravis, insulin resistance, rheumatoid arthritis, scleroderma, and autoimmune infertility), tumorigenesis, and tumor metastasis.

The method includes one or more of the following:

(i) detecting, in a tissue of the subject, the presence or absence of a mutation which affects the expression of the 47169/33935 gene, or detecting the presence or absence of a mutation in a region which controls the expression of the gene, e.g., a mutation in the 5′-control region;

(ii) detecting, in a tissue of the subject, the presence or absence of a mutation which alters the structure of the 47169/33935 gene;

(iii) detecting, in a tissue of the subject, the malexpression of the 47169/33935 gene at the mRNA level, e.g., detecting a non-wild-type level of a mRNA; and

(iv) detecting, in a tissue of the subject, the malexpression of the gene at the protein level, e.g., detecting a non-wild-type level of a 47169/33935 polypeptide.

In preferred embodiments the method includes: ascertaining the existence of at least one of: a deletion of one or more nucleotides from the 47169/33935 gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from one of SEQ ID NOs: 1 and 11, or naturally occurring mutants thereof, or 5′- or 3′-flanking sequences naturally associated with the 47169/33935 gene; (ii) exposing the probe/primer to nucleic acid of the tissue; and detecting the presence or absence of the genetic lesion by hybridization of the probe/primer to the nucleic acid, e.g., by in situ hybridization.

In preferred embodiments, detecting the malexpression includes ascertaining the existence of at least one of: an alteration in the level of a messenger RNA transcript of the 47169/33935 gene; the presence of a non-wild-type splicing pattern of a messenger RNA transcript of the gene; or a non-wild-type level of 47169/33935 RNA or protein.

Methods of the invention can be used for prenatal screening or to determine if a subject's offspring will be at risk for a disorder.

In preferred embodiments the method includes determining the structure of a 47169/33935 gene, an abnormal structure being indicative of risk for the disorder.

In preferred embodiments the method includes contacting a sample form the subject with an antibody to the 47169/33935 protein or a nucleic acid, which hybridizes specifically with the gene. These and other embodiments are discussed below.

Diagnostic and Prognostic Assays

The presence, level, or absence of 47169/33935 protein or nucleic acid in a biological sample can be evaluated by obtaining a biological sample from a test subject and contacting the biological sample with a compound or an agent capable of detecting 47169/33935 protein or nucleic acid (e.g., mRNA, genomic DNA) that encodes 47169/33935 protein such that the presence of 47169/33935 protein or nucleic acid is detected in the biological sample. The term “biological sample” includes tissues, cells and biological fluids isolated from a subject, as well as tissues, cells and fluids present within a subject. A preferred biological sample is serum. The level of expression of the 47169/33935 gene can be measured in a number of ways, including, but not limited to: measuring the mRNA encoded by the 47169/33935 genes; measuring the amount of protein encoded by the 47169/33935 genes; or measuring the activity of the protein encoded by the 47169/33935 genes.

The level of mRNA corresponding to the 47169/33935 gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length 47169/33935 nucleic acid, such as the nucleic acid of one of SEQ ID NOs: 1 and 11, or a portion thereof, such as an oligonucleotide of at least 7, 15, 30, 50, 100, 250 or 500 nucleotides in length and sufficient to specifically hybridize under stringent conditions to 47169/33935 mRNA or genomic DNA. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array. A skilled artisan can adapt known mRNA detection methods for use in detecting the level of mRNA encoded by the 47169/33935 genes.

The level of mRNA in a sample that is encoded by 47169/33935 can be evaluated with nucleic acid amplification, e.g., by RT-PCR (U.S. Pat. No. 4,683,202), ligase chain reaction (Barany, 1991, Proc. Natl. Acad. Sci. USA 88:189-193), self-sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′- or 3′-regions of a 47169/33935 gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence between the primers.

For in situ methods, a cell or tissue sample can be prepared/processed and immobilized on a support, typically a glass slide, and then contacted with a probe that can hybridize to mRNA that encodes the 47169/33935 gene being analyzed.

In another embodiment, the methods include further contacting a control sample with a compound or agent capable of detecting 47169/33935 mRNA, or genomic DNA, and comparing the presence of 47169/33935 mRNA or genomic DNA in the control sample with the presence of 47169/33935 mRNA or genomic DNA in the test sample.

A variety of methods can be used to determine the level of protein encoded by 47169/33935. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect 47169/33935 protein in a biological sample in vitro as well as in vivo. In vitro techniques for detection of 47169/33935 protein include enzyme linked immunosorbent assays (ELISAs), immunoprecipitations, immunofluorescence, enzyme immunoassay (EIA), radioimmunoassay (RIA), and Western blot analysis. In vivo techniques for detection of 47169/33935 protein include introducing into a subject a labeled anti-47169/33935 antibody. For example, the antibody can be labeled with a radioactive marker whose presence and location in a subject can be detected by standard imaging techniques.

In another embodiment, the methods further include contacting the control sample with a compound or agent capable of detecting 47169/33935 protein, and comparing the presence of 47169/33935 protein in the control sample with the presence of 47169/33935 protein in the test sample.

The invention also includes kits for detecting the presence of 47169/33935 in a biological sample. For example, the kit can include a compound or agent capable of detecting 47169/33935 protein or mRNA in a biological sample, and a standard. The compound or agent can be packaged in a suitable container. The kit can further comprise instructions for using the kit to detect 47169/33935 protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably-labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein-stabilizing agent. The kit can also includes components necessary for detecting the detectable agent (e.g., an enzyme or a substrate). The kit can also contain a control sample or a series of control samples that can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with malexpressed, aberrant or unwanted 47169/33935 expression or activity. As used herein, the term “unwanted” includes an unwanted phenomenon involved in a biological response such as induction of an inappropriate immune response or deregulated cell proliferation.

In one embodiment, a disease or disorder associated with aberrant or unwanted 47169/33935 expression or activity is identified. A test sample is obtained from a subject and 47169/33935 protein or nucleic acid (e.g., mRNA or genomic DNA) is evaluated, wherein the level, e.g., the presence or absence, of 47169/33935 protein or nucleic acid is diagnostic for a subject having or at risk of developing a disease or disorder associated with aberrant or unwanted 47169/33935 expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 47169/33935 expression or activity. For example, such methods can be used to determine whether a subject can be effectively treated with an agent that modulates 47169/33935 expression or activity.

The methods of the invention can also be used to detect genetic alterations in a 47169/33935 gene, thereby determining if a subject with the altered gene is at risk for a disorder characterized by misregulation in 47169/33935 protein activity or nucleic acid expression, such as a disorder associated with tumorigenesis or induction of an inappropriate immune response. In preferred embodiments, the methods include detecting, in a sample obtained from the subject, the presence or absence of a genetic alteration characterized by at least one of an alteration affecting the integrity of a gene encoding a 47169/33935 protein, or the malexpression of the 47169/33935 gene. For example, such genetic alterations can be detected by ascertaining the existence of at least one of 1) a deletion of one or more nucleotides from a 47169/33935 gene; 2) an addition of one or more nucleotides to a 47169/33935 gene; 3) a substitution of one or more nucleotides of a 47169/33935 gene, 4) a chromosomal rearrangement of a 47169/33935 gene; 5) an alteration in the level of a messenger RNA transcript of a 47169/33935 gene, 6) aberrant modification of a 47169/33935 gene, such as of the methylation pattern of the genomic DNA, 7) the presence of a non-wild-type splicing pattern of a messenger RNA transcript of a 47169/33935 gene, 8) a non-wild-type level of a 47169/33935 protein, 9) allelic loss of a 47169/33935 gene, and 10) inappropriate post-translational modification of a 47169/33935 protein.

An alteration can be detected without a probe/primer in a polymerase chain reaction, such as anchor PCR or RACE-PCR, or, alternatively, in a ligation chain reaction (LCR), the latter of which can be particularly useful for detecting point mutations in the 47169/33935 gene. This method can include the steps of collecting a sample of cells from a subject, isolating nucleic acid (e.g., genomic, mRNA or both) from the sample, contacting the nucleic acid sample with one or more primers which specifically hybridize to a 47169/33935 gene under conditions such that hybridization and amplification of the 47169/33935 gene occurs (if present), and detecting the presence or absence of an amplification product, or detecting the size of the amplification product and comparing the length to a control sample. It is anticipated that PCR and/or LCR can be desirable to use as a preliminary amplification step in conjunction with any of the techniques used for detecting mutations described herein.

Alternative amplification methods include: self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), or other nucleic acid amplification methods, followed by the detection of the amplified molecules using techniques known to those of skill in the art.

In another embodiment, mutations in a 47169/33935 gene from, a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis, and compared. Differences in fragment length sizes between sample and control DNA indicates mutations in the sample DNA. Moreover, the use of sequence specific ribozymes (e.g., U.S. Pat. No. 5,498,531) can be used to score for the presence of specific mutations by development or loss of a ribozyme cleavage site.

In other embodiments, genetic mutations in 47169/33935 can be identified by hybridizing a sample to control nucleic acids, e.g., DNA or RNA, by, e.g., two-dimensional arrays, or, e.g., chip based arrays. Such arrays include a plurality of addresses, each of which is positionally distinguishable from the other. A different probe is located at each address of the plurality. The arrays can have a high density of addresses, e.g., can contain hundreds or thousands of oligonucleotides probes (Cronin et al., 1996, Hum. Mutat. 7:244-255; Kozal et al., 1996, Nature Med. 2:753-759). For example, genetic mutations in 47169/33935 can be identified in two-dimensional arrays containing light-generated DNA probes as described (Cronin et al., supra). Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 47169/33935 gene and detect mutations by comparing the sequence of the sample 47169/33935 with the corresponding wild-type (control) sequence. Automated sequencing procedures can be utilized when performing the diagnostic assays (1995, Biotechniques 19:448), including sequencing by mass spectrometry.

Other methods for detecting mutations in the 47169/33935 gene include methods in which protection from cleavage agents is used to detect mismatched bases in RNA/RNA or RNA/DNA heteroduplexes (Myers et al., 1985, Science 230:1242; Cotton et al., 1988, Proc. Natl. Acad. Sci. USA 85:4397; Saleeba et al., 1992, Meth. Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in 47169/33935 cDNAs obtained from samples of cells. For example, the mutY enzyme of E. coli cleaves A at G/A mismatches and the thymidine DNA glycosylase from HeLa cells cleaves T at G/T mismatches (Hsu et al., 1994, Carcinogenesis 15:1657-1662; U.S. Pat. No. 5,459,039).

In other embodiments, alterations in electrophoretic mobility will be used to identify mutations in 47169/33935 genes. For example, single strand conformation polymorphism (SSCP) can be used to detect differences in electrophoretic mobility between mutant and wild-type nucleic acids (Orita et al., 1989, Proc. Natl. Acad. Sci. USA 86:2766; Cotton, 1993, Mutat. Res. 285:125-144; Hayashi, 1992, Genet. Anal. Tech. Appl. 9:73-79). Single-stranded DNA fragments of sample and control 47169/33935 nucleic acids will be denatured and allowed to re-nature. The secondary structure of single-stranded nucleic acids varies according to sequence, the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al., 1991, Trends Genet 7:5).

In yet another embodiment, the movement of mutant or wild-type fragments in polyacrylamide gels containing a gradient of denaturant is assayed using denaturing gradient gel electrophoresis (DGGE) (Myers et al., 1985, Nature 313:495). When DGGE is used as the method of analysis, DNA will be modified to insure that it does not completely denature, for example by adding a GC clamp of approximately 40 base pairs of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al., 1986, Nature 324:163; Saiki et al., 1989, Proc. Natl. Acad. Sci. USA 86:6230).

Alternatively, allele specific amplification technology that depends on selective PCR amplification can be used in conjunction with the instant invention. Oligonucleotides used as primers for specific amplification can carry the mutation of interest in the center of the molecule (so that amplification depends on differential hybridization; Gibbs et al., 1989, Nucl. Acids Res. 17:2437-2448) or at the extreme 3′-end of one primer where, under appropriate conditions, mismatch can prevent, or reduce polymerase extension (Prossner, 1993, Tibtech 11:238). In addition, it can be desirable to introduce a novel restriction site in the region of the mutation to create cleavage-based detection (Gasparini et al., 1992, Mol. Cell Probes 6:1). It is anticipated that in certain embodiments, amplification can also be performed using Taq ligase for amplification (Barany, 1991, Proc. Natl. Acad. Sci. USA 88:189). In such cases, ligation will occur only if there is a perfect match at the 3′-end of the 5′-sequence making it possible to detect the presence of a known mutation at a specific site by looking for the presence or absence of amplification.

The methods described herein can be performed, for example, using pre-packaged diagnostic kits comprising at least one probe nucleic acid or antibody reagent described herein, which can be conveniently used, e.g., in clinical settings to diagnose patients exhibiting symptoms or family history of a disease or illness involving a 47169/33935 gene.

Use of 47169/33935 Molecules as Surrogate Markers

The 47169/33935 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 47169/33935 molecules of the invention can be detected, and can be correlated with one or more biological states in vivo. For example, the 47169/33935 molecules of the invention can serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers can serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease can be made using cholesterol levels as a surrogate marker, and an analysis of HIV infection can be made using HIV RNA levels as a surrogate marker, well in advance of the undesirable clinical outcomes of myocardial infarction or fully-developed AIDS). Examples of the use of surrogate markers have been described (e.g., Koomen et al., 2000, J. Mass. Spectrom. 35:258-264; James, 1994, AIDS Treat. News Arch. 209).

The 47169/33935 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker can be indicative of the concentration of the drug in a biological tissue, in that the marker is either expressed or transcribed or not expressed or transcribed in that tissue in relationship to the level of the drug. In this fashion, the distribution or uptake of the drug can be monitored by the pharmacodynamic marker. Similarly, the presence or quantity of the pharmacodynamic marker can be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug can be sufficient to activate multiple rounds of marker (e.g., a 47169/33935 marker) transcription or expression, the amplified marker can be in a quantity which is more readily detectable than the drug itself. Also, the marker can be more easily detected due to the nature of the marker itself; for example, using the methods described herein, anti-47169/33935 antibodies can be employed in an immune-based detection system for a 47169/33935 protein marker, or 47169/33935-specific radiolabeled probes can be used to detect a 47169/33935 mRNA marker. Furthermore, the use of a pharmacodynamic marker can offer mechanism-based prediction of risk due to drug treatment beyond the range of possible direct observations. Examples of the use of pharmacodynamic markers have been described (e.g., U.S. Pat. No. 6,033,862; Hattis et al., 1991, Env. Health Perspect. 90: 229-238; Schentag, 1999, Am. J. Health-Syst. Pharm. 56 Suppl. 3: S21-S24; Nicolau, 1999, Am, J. Health-Syst. Pharm. 56 Suppl. 3: S16-S20).

The 47169/33935 molecules of the invention are also useful as pharmacogenomic markers. As used herein, a “pharmacogenomic marker” is an objective biochemical marker which correlates with a specific clinical drug response or susceptibility in a subject (e.g., McLeod et al., 1999, Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, can be selected. For example, based on the presence or quantity of RNA, or protein (e.g., 47169/33935 protein or RNA) for specific tumor markers in a subject, a drug or course of treatment can be selected that is optimized for the treatment of the specific tumor likely to be present in the subject. Similarly, the presence or absence of a specific sequence mutation in 47169/33935 DNA can correlate 47169/33935 drug response. The use of pharmacogenomic markers therefore permits the application of the most appropriate treatment for each subject without having to administer the therapy.

Pharmaceutical Compositions

The nucleic acid and polypeptides, fragments thereof, as well as anti-47169/33935 antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; anti-bacterial agents such as benzyl alcohol or methyl parabens; anti-oxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including an agent in the composition that delays absorption, for example, aluminum monostearate or gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle that contains a basic dispersion medium and the other required ingredients from those enumerated above. In the case of sterile powders for the preparation of sterile injectable solutions, the preferred methods of preparation are vacuum drying and freeze-drying, which yield a powder comprising the active ingredient and any additional desired ingredient from a previously sterile filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents and/or adjuvant materials can be included as part of the composition. The tablets, pills, capsules, troches and the like can contain any of the following ingredients, or compounds of a similar nature: a binder, such as microcrystalline cellulose, gum tragacanth or gelatin; an excipient, such as starch or lactose; a disintegrating agent, such as alginic acid, Primogel™, or corn starch; a lubricant, such as magnesium stearate or Sterotes™; a glidant, such as colloidal silicon dioxide; a sweetening agent, such as sucrose or saccharin; or a flavoring agent, such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished using nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells using monoclonal antibodies directed towards viral antigens) can also be used as pharmaceutically acceptable carriers. These can be prepared according to described methods (e.g., U.S. Pat. No. 4,522,811).

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of protein or polypeptide (i.e., an effective dosage) ranges from about 0.001 to 30 milligrams per kilogram body weight, preferably about 0.01 to 25 milligrams per kilogram body weight, more preferably about 0.1 to 20 milligrams per kilogram body weight, and even more preferably about 1 to 10 milligrams per kilogram, 2 to 9 milligrams per kilogram, 3 to 8 milligrams per kilogram, 4 to 7 milligrams per kilogram, or 5 to 6 milligrams per kilogram body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 milligrams per kilogram of body weight (generally 10 to 20 milligrams per kilogram). If the antibody is to act in the brain, a dosage of 50 to 100 milligrams per kilogram is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidating antibodies is described by Cruikshank et al. (1997, J. AIDS Hum. Retrovir. 14:193).

The present invention encompasses agents that modulate expression or activity of a 47169/33935 protein or nucleic acid. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including hetero-organic and organo-metallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Examples of suitable doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody (or fragment thereof) can be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, and the drug moiety is not to be construed as limited to classical chemical therapeutic agents. For example, the drug moiety can be a protein or polypeptide possessing a desired biological activity. Such proteins can include, for example, a toxin such as abrin, ricin A, gelonin, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukins-1, -2, and -6, granulocyte macrophage colony stimulating factor, granulocyte colony stimulating factor, or other growth factors.

Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (e.g., Chen. et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of Treatment

The present invention provides for both prophylactic and therapeutic methods of treating a subject at risk of (or susceptible to) a disorder or having a disorder associated with aberrant or unwanted 47169/33935 expression or activity. With regards to both prophylactic and therapeutic methods of treatment, such treatments can be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype,” or “drug response genotype”.) Thus, another aspect of the invention provides methods for tailoring an individual's prophylactic or therapeutic treatment with either the 47169/33935 molecules of the present invention or 47169/33935 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Treatment is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

A therapeutic agent includes, but is not limited to, small molecules, peptides, antibodies, ribozymes and antisense oligonucleotides.

In one aspect, the invention provides a method for preventing a disease or condition in a subject associated with an aberrant or unwanted 47169/33935 expression or activity, by administering to the subject a 47169/33935 or an agent which modulates 47169/33935 expression, or at least one 47169/33935 activity. Subjects at risk for a disease which is caused or contributed to by aberrant or unwanted 47169/33935 expression or activity can be identified by, for example, any or a combination of diagnostic or prognostic assays as described herein. Administration of a prophylactic agent can occur prior to the manifestation of symptoms characteristic of the 47169/33935 aberrance, such that a disease or disorder is prevented or, alternatively, delayed in its progression. Depending on the type of 47169/33935 aberrance, for example, a 47169/33935 protein, 47169/33935 agonist or 47169/33935 antagonist agent can be used for treating the subject. The appropriate agent can be determined based on screening assays described herein.

It is possible that some 47169/33935 disorders can be caused, at least in part, by an abnormal level of gene product, or by the presence of a gene product exhibiting abnormal activity. As such, the reduction in the level and/or activity of such gene products would bring about the amelioration of disorder symptoms.

As discussed, successful treatment of 47169/33935 disorders can be brought about by techniques that inhibit expression or activity of target gene products. For example, compounds, e.g., an agent identified using an assays described above, that proves to exhibit negative modulatory activity, can be used in accordance with the invention to prevent and/or ameliorate symptoms of 47169/33935 disorders. Such molecules can include, but are not limited to peptides, phosphopeptides, small organic or inorganic molecules, or antibodies (including, for example, polyclonal, monoclonal, humanized, anti-idiotypic, chimeric or single chain antibodies, and Fab, F(ab′)₂ and Fab expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules can be utilized in treating or preventing a disease characterized by 47169/33935 expression is through the use of aptamer molecules specific for 47169/33935 protein. Aptamers are nucleic acid molecules having a tertiary structure that permits them to specifically bind to protein ligands (e.g., Osborne et al., 1997, Curr. Opin. Chem. Biol. 1:5-9; Patel, 1997, Curr. Opin. Chem. Biol. 1:32-46). Since nucleic acid molecules can in many cases be more conveniently introduced into target cells than therapeutic protein molecules can be, aptamers offer a method by which 47169/33935 protein activity can be specifically decreased without the introduction of drugs or other molecules which can have pluripotent effects.

Antibodies can be generated that are both specific for target gene product and that reduce target gene product activity. Such antibodies may, therefore, by administered in instances whereby negative modulatory techniques are appropriate for the treatment of 47169/33935 disorders.

In circumstances wherein injection of an animal or a human subject with a 47169/33935 protein or epitope for stimulating antibody production is harmful to the subject, it is possible to generate an immune response against 47169/33935 through the use of anti-idiotypic antibodies (e.g., Herlyn, 1999, Ann. Med. 31:66-78; Bhattacharya-Chatterjee et al., 1998, Cancer Treat. Res. 94:51-68). If an anti-idiotypic antibody is introduced into a mammal or human subject, it should stimulate the production of anti-anti-idiotypic antibodies, which should be specific to the 47169/33935 protein. Vaccines directed to a disease characterized by 47169/33935 expression can also be generated in this fashion.

In instances where the target antigen is intracellular and whole antibodies are used, internalizing antibodies can be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (e.g., Marasco et al., 1993, Proc. Natl. Acad. Sci. USA 90:7889-7893).

The identified compounds that inhibit target gene expression, synthesis and/or activity can be administered to a patient at therapeutically effective doses to prevent, treat or ameliorate 47169/33935 disorders. A therapeutically effective dose refers to that amount of the compound sufficient to result in amelioration of symptoms of the disorders.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays can utilize antibody mimics and/or “biosensors” that have been created through molecular imprinting techniques. The compound which is able to modulate 47169/33935 activity is used as a template, or “imprinting molecule,” to spatially organize polymerizable monomers prior to their polymerization with catalytic reagents. The subsequent removal of the imprinted molecule leaves a polymer matrix that contains a repeated “negative image” of the compound and is able to selectively rebind the molecule under biological assay conditions. Detailed reviews of this technique appear in the art (Ansell et al., 1996, Curr. Opin. Biotechnol. 7:89-94; Shea, 1994, Trends Polymer Sci. 2:166-173). Such “imprinted” affinity matrixes are amenable to ligand-binding assays, whereby the immobilized monoclonal antibody component is replaced by an appropriately imprinted matrix (e.g., a matrix described in Vlatakis et al., 1993, Nature 361:645-647. Through the use of isotope-labeling, the “free” concentration of compound which modulates the expression or activity of 47169/33935 can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiber optic devices, in turn allowing the dose in a test subject to be quickly optimized based on its individual IC₅₀. A rudimentary example of such a “biosensor” is discussed in Kriz et al. (1995, Anal. Chem. 67:2142-2144).

Another aspect of the invention pertains to methods of modulating 47169/33935 expression or activity for therapeutic purposes. Accordingly, in an exemplary embodiment, the modulatory method of the invention involves contacting a cell with a 47169/33935 or agent that modulates one or more of the activities of 47169/33935 protein activity associated with the cell. An agent that modulates 47169/33935 protein activity can be an agent as described herein, such as a nucleic acid or a protein, a naturally-occurring target molecule of a 47169/33935 protein (e.g., a 47169/33935 substrate or receptor), a 47169/33935 antibody, a 47169/33935 agonist or antagonist, a peptidomimetic of a 47169/33935 agonist or antagonist, or other small molecule.

In one embodiment, the agent stimulates one or more 47169/33935 activities. Examples of such stimulatory agents include active 47169/33935 protein and a nucleic acid molecule encoding 47169/33935. In another embodiment, the agent inhibits one or more 47169/33935 activities. Examples of such inhibitory agents include antisense 47169/33935 nucleic acid molecules, anti-47169/33935 antibodies, and 47169/33935 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 47169/33935 protein or nucleic acid molecule. In one embodiment, the method involves administering an agent (e.g., an agent identified by a screening assay described herein), or combination of agents that modulates (e.g., up-regulates or down-regulates) 47169/33935 expression or activity. In another embodiment, the method involves administering a 47169/33935 protein or nucleic acid molecule as therapy to compensate for reduced, aberrant, or unwanted 47169/33935 expression or activity.

Stimulation of 47169/33935 activity is desirable in situations in which 47169/33935 is abnormally down-regulated and/or in which increased 47169/33935 activity is likely to have a beneficial effect. For example, stimulation of 47169/33935 activity is desirable in situations in which a 47169/33935 is down-regulated and/or in which increased 47169/33935 activity is likely to have a beneficial effect. Likewise, inhibition of 47169/33935 activity is desirable in situations in which 47169/33935 is abnormally up-regulated and/or in which decreased 47169/33935 activity is likely to have a beneficial effect.

Pharmacogenomics

The 47169/33935 molecules of the present invention, as well as agents, or modulators which have a stimulatory or inhibitory effect on 47169/33935 activity (e.g., 47169/33935 gene expression) as identified by a screening assay described herein can be administered to individuals to treat (prophylactically or therapeutically) 47169/33935-associated disorders associated with aberrant or unwanted 47169/33935 activity (e.g., disorders associated with tumorigenesis, tumor metastasis, or induction of an inappropriate immune response). In conjunction with such treatment, pharmacogenomics (i.e., the study of the relationship between an individual's genotype and that individual's response to a foreign compound or drug) can be considered. Differences in metabolism of therapeutics can lead to severe toxicity or therapeutic failure by altering the relation between dose and blood concentration of the pharmacologically active drug. Thus, a physician or clinician can consider applying knowledge obtained in relevant pharmacogenomics studies in determining whether to administer a 47169/33935 molecule or 47169/33935 modulator as well as tailoring the dosage and/or therapeutic regimen of treatment with a 47169/33935 molecule or 47169/33935 modulator.

Pharmacogenomics deals with clinically significant hereditary variations in the response to drugs due to altered drug disposition and abnormal action in affected persons (e.g., Eichelbaum et al., 1996, Clin. Exp. Pharmacol. Physiol. 23:983-985; Linder et al., 1997, Clin. Chem. 43:254-266). In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is hemolysis after ingestion of oxidant drugs (anti-malarials, sulfonamides, analgesics, nitrofurans) and consumption of fava beans.

One pharmacogenomics approach to identifying genes that predict drug response, known as “a genome-wide association,” relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high-resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP can be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that can be common among such genetically similar individuals.

Alternatively, a method termed the “candidate gene approach” can be utilized to identify genes that predict drug response. According to this method, if a gene that encodes a drug's target is known (e.g., a 47169/33935 protein of the present invention), all common variants of that gene can be fairly easily identified in the population and it can be determined if having one version of the gene versus another is associated with a particular drug response.

Alternatively, a method termed “gene expression profiling,” can be utilized to identify genes that predict drug response. For example, the gene expression of an animal dosed with a drug (e.g., a 47169/33935 molecule or 47169/33935 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a 47169/33935 molecule or 47169/33935 modulator, such as a modulator identified by one of the exemplary screening assays described herein.

The present invention further provides methods for identifying new agents, or combinations, that are based on identifying agents that modulate the activity of one or more of the gene products encoded by one or more of the 47169/33935 genes of the present invention, wherein these products can be associated with resistance of the cells to a therapeutic agent. Specifically, the activity of the proteins encoded by the 47169/33935 genes of the present invention can be used as a basis for identifying agents for overcoming agent resistance. By blocking the activity of one or more of the resistance proteins, target cells, e.g., cells of the immune system, will become sensitive to treatment with an agent that the unmodified target cells were resistant to.

Monitoring the influence of agents (e.g., drugs) on the expression or activity of a 47169/33935 protein can be applied in clinical trials. For example, the effectiveness of an agent determined by a screening assay as described herein to increase 47169/33935 gene expression, protein levels, or up-regulate 47169/33935 activity, can be monitored in clinical trials of subjects exhibiting decreased 47169/33935 gene expression, protein levels, or down-regulated 47169/33935 activity. Alternatively, the effectiveness of an agent determined by a screening assay to decrease 47169/33935 gene expression, protein levels, or down-regulate 47169/33935 activity, can be monitored in clinical trials of subjects exhibiting increased 47169/33935 gene expression, protein levels, or up-regulated 47169/33935 activity. In such clinical trials, the expression or activity of a 47169/33935 gene, and preferably, other genes that have been implicated in, for example, a 47169/33935-associated disorder can be used as a “read out” or markers of the phenotype of a particular cell.

Other Embodiments

In another aspect, the invention features, a method of analyzing a plurality of capture probes. The method can be used, e.g., to analyze gene expression. The method includes: providing a two-dimensional array having a plurality of addresses, each address of the plurality being positionally distinguishable from each other address of the plurality, and each address of the plurality having a unique capture probe, e.g., a nucleic acid or peptide sequence; contacting the array with a 47169/33935, preferably purified, nucleic acid, preferably purified, polypeptide, preferably purified, or antibody, and thereby evaluating the plurality of capture probes. Binding, e.g., in the case of a nucleic acid, hybridization with a capture probe at an address of the plurality, is detected, e.g., by signal generated from a label attached to the 47169/33935 nucleic acid, polypeptide, or antibody.

The capture probes can be a set of nucleic acids from a selected sample, e.g., a sample of nucleic acids derived from a control or non-stimulated tissue or cell.

The method can include contacting the 47169/33935 nucleic acid, polypeptide, or antibody with a first array having a plurality of capture probes and a second array having a different plurality of capture probes. The results of hybridization can be compared, e.g., to analyze differences in expression between a first and second sample. The first plurality of capture probes can be from a control sample, e.g., a wild-type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of 47169/33935. Such methods can be used to diagnose a subject, e.g., to evaluate risk for a disease or disorder, to evaluate suitability of a selected treatment for a subject, to evaluate whether a subject has a disease or disorder. 47169 and 33935 are associated with protein and lipid glycosylation, thus it is useful for evaluating disorders relating to aberrant protein and lipid glycosylation, such as tumorigenesis and inappropriate immune responses.

The method can be used to detect SNPs, as described above.

In another aspect, the invention features, a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express 47169/33935 or from a cell or subject in which a 47169/33935 mediated response has been elicited, e.g., by contact of the cell with 47169/33935 nucleic acid or protein, or administration to the cell or subject 47169/33935 nucleic acid or protein; contacting the array with one or more inquiry probe, wherein an inquiry probe can be a nucleic acid, polypeptide, or antibody (which is preferably other than 47169/33935 nucleic acid, polypeptide, or antibody); providing a two-dimensional array having a plurality of addresses, each address of the plurality being positionally distinguishable from each other address of the plurality, and each address of the plurality having a unique capture probe, e.g., wherein the capture probes are from a cell or subject which does not express 47169/33935 (or does not express as highly as in the case of the 47169/33935 positive plurality of capture probes) or from a cell or subject which in which a 47169/33935 mediated response has not been elicited (or has been elicited to a lesser extent than in the first sample); contacting the array with one or more inquiry probes (which is preferably other than a 47169/33935 nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features, a method of analyzing a plurality of probes or a sample. The method is useful, e.g., for analyzing gene expression. The method includes: providing a two dimensional array having a plurality of addresses, each address of the plurality being positionally distinguishable from each other address of the plurality having a unique capture probe, contacting the array with a first sample from a cell or subject which express or malexpress 47169/33935 or from a cell or subject in which a 47169/33935-mediated response has been elicited, e.g., by contact of the cell with 47169/33935 nucleic acid or protein, or administration to the cell or subject 47169/33935 nucleic acid or protein; providing a two dimensional array having a plurality of addresses, each address of the plurality being positionally distinguishable from each other address of the plurality, and each address of the plurality having a unique capture probe, and contacting the array with a second sample from a cell or subject which does not express 47169/33935 (or does not express as highly as in the case of the 47169/33935 positive plurality of capture probes) or from a cell or subject which in which a 47169/33935 mediated response has not been elicited (or has been elicited to a lesser extent than in the first sample); and comparing the binding of the first sample with the binding of the second sample. Binding, e.g., in the case of a nucleic acid, hybridization with a capture probe at an address of the plurality, is detected, e.g., by signal generated from a label attached to the nucleic acid, polypeptide, or antibody. The same array can be used for both samples or different arrays can be used. If different arrays are used the plurality of addresses with capture probes should be present on both arrays.

In another aspect, the invention features a method of analyzing 47169/33935, e.g., analyzing structure, function, or relatedness to other nucleic acid or amino acid sequences. The method includes: providing a 47169/33935 nucleic acid or amino acid sequence, e.g., nucleotide sequence from 47169/33935 or a portion thereof; comparing the 47169/33935 sequence with one or more preferably a plurality of sequences from a collection of sequences, e.g., a nucleic acid or protein sequence database; to thereby analyze 47169/33935.

The method can include evaluating the sequence identity between a 47169/33935 sequence and a database sequence. The method can be performed by accessing the database at a second site, e.g., via the internet.

In another aspect, the invention features, a set of oligonucleotides, useful, e.g., for identifying SNPs, or identifying specific alleles of 47169/33935. The set includes a plurality of oligonucleotides, each of which has a different nucleotide at an interrogation position, e.g., an SNP or the site of a mutation. In a preferred embodiment, the plurality of oligonucleotides are identical in sequence with one another (except for differences in length). The oligonucleotides can be provided with differential labels, such that an oligonucleotide that hybridizes to one allele provides a signal that is distinguishable from an oligonucleotide that hybridizes to a second allele.

The sequence of a 47169/33935 molecules can be provided in a variety of media to facilitate use thereof. A sequence can be provided as a manufacture, other than an isolated nucleic acid or amino acid molecule, which contains sequence information for a 47169/33935 nucleic acid or protein. Such a manufacture can provide a nucleotide or amino acid sequence, e.g., an open reading frame, in a form which allows examination of the manufacture using means not directly applicable to examining the nucleotide or amino acid sequences, or a subset thereof, as they exists in nature or in purified form.

A 47169/33935 nucleotide or amino acid sequence can be recorded on computer readable media. As used herein, “computer readable media” refers to any medium that can be read and accessed directly by a computer. Such media include, but are not limited to: magnetic storage media, such as floppy discs, hard disc storage media, and magnetic tape; optical storage media such as CD-ROM; electrical storage media such as RAM and ROM; and hybrids of these categories such as magnetic/optical storage media.

A variety of data storage structures are available to a skilled artisan for creating a computer readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect™ and Microsoft Word™, or represented in the form of an ASCII file, stored in a database application, such as DB2, Sybase™, Oracle™, or the like. The skilled artisan can readily adapt any number of data processor structuring formats (e.g., text file or database) in order to obtain computer readable medium having recorded thereon the nucleotide sequence information of the present invention.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. A search is used to identify fragments or regions of the sequences of the invention that match a particular target sequence or target motif.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. Typical sequence lengths of a target sequence are from about 10 to 100 amino acids or from about 30 to 300 nucleotide residues. However, it is well recognized that commercially important fragments, such as sequence fragments involved in gene expression and protein processing, can be of shorter length.

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBIA).

Thus, the invention features a method of making a computer readable record of a sequence of a 47169/33935 sequence that includes recording the sequence on a computer readable matrix. In a preferred embodiment, the record includes one or more of the following: identification of an open reading frame; identification of a domain, region, or site; identification of the start of transcription; identification of the transcription terminator; the full length amino acid sequence of the protein, or a mature form thereof; the 5′-end of the translated region; or 5′- and/or 3′-regulatory regions.

In another aspect, the invention features, a method of analyzing a sequence. The method includes: providing a 47169/33935 sequence or record, in computer readable form; comparing a second sequence to the gene name sequence; thereby analyzing a sequence. Comparison can include comparing to sequences for sequence identity or determining if one sequence is included within the other, e.g., determining if the 47169/33935 sequence includes a sequence being compared. In a preferred embodiment, the 47169/33935 or second sequence is stored on a first computer, e.g., at a first site and the comparison is performed, read, or recorded on a second computer, e.g., at a second site. E.g., the 47169/33935 or second sequence can be stored in a public or proprietary database in one computer, and the results of the comparison performed, read, or recorded on a second computer. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′-end of the translated region; or 5′- and/or 3′-regulatory regions.

This invention is further illustrated by the following examples that should not be construed as limiting. The contents of all references, patents and published patent applications cited throughout this application are incorporated herein by reference.

### EXAMPLES

### Example 1

Identification and Characterization of Human 47169 and 33935 cDNAs

The human 47169 nucleotide sequence (FIG. 1; SEQ ID NO: 1), which is approximately 3985 nucleotides in length including non-translated regions, contains a predicted methionine-initiated coding sequence at about nucleotide residues 98-1906. The coding sequence encodes a 603 amino acid protein (SEQ ID NO: 2).

The human 33935 nucleotide sequence (FIG. 3; SEQ ID NO: 11), which is approximately 2590 nucleotides in length including non-translated regions, contains a predicted methionine-initiated coding sequence at about nucleotide residues 11-1486. The coding sequence encodes a 492 amino acid protein (SEQ ID NO: 12).

### Example 2

Tissue Distribution of 47169/33935 mRNA

Northern blot hybridizations with various RNA samples can be performed under standard conditions and washed under stringent conditions, i.e., 0.2×SSC at 65° C. A DNA probe corresponding to all or a portion of the 47169/33935 cDNA (SEQ ID NO: 1) can be used. The DNA can, for example, be radioactively labeled with ³²P-dCTP using the Prime-It™ Kit (Stratagene, La Jolla, Calif.) according to the instructions of the supplier. Filters containing mRNA from mouse hematopoietic and endocrine tissues, and cancer cell lines (Clontech, Palo Alto, Calif.) can be probed in ExpressHyb™ hybridization solution (Clontech) and washed at high stringency according to manufacturer's recommendations.

### Example 3

Recombinant Expression of 47169/33935 in Bacterial Cells

In this example, 47169/33935 is expressed as a recombinant glutathione-S-transferase (GST) fusion polypeptide in E. coli and the fusion polypeptide is isolated and characterized. Specifically, 47169/33935 nucleic acid sequences are fused to GST nucleic acid sequences and this fusion construct is expressed in E. coli, e.g., strain PEB199. Expression of the GST-47169/33935 fusion construct in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 4

Expression of Recombinant 47169/33935 Protein in COS Cells

To express the 47169/33935 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 47169/33935 protein and an HA tag (Wilson et al., 1984, Cell 37:767) or a FLAG® tag fused in-frame to its 3′-end of the fragment is cloned into the polylinker region of the vector, thereby placing the expression of the recombinant protein under the control of the CMV promoter.

To construct the plasmid, the 47169/33935 DNA sequence is amplified by PCR using two primers. The 5′ primer contains the restriction site of interest followed by approximately twenty nucleotides of the 47169/33935 coding sequence starting from the initiation codon; the 3′-end sequence contains complementary sequences to the other restriction site of interest, a translation stop codon, the HA tag or FLAG® tag and the last 20 nucleotides of the 47169/33935 coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the 47169/33935 gene is inserted in the desired orientation. The ligation mixture is transformed into E. coli cells (strains HB101, DH5alpha, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 47169/33935-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook et al., (1989, Molecular Cloning: A Laboratory Manual. 2nd ed., Cold Spring Harbor Laboratory Press, Cold Spring Harbor, N.Y.). The expression of the 47169/33935 polypeptide is detected by radiolabeling (³⁵S-methionine or ³⁵S-cysteine, available from NEN, Boston, Mass., can be used) and immunoprecipitation (Harlow et al., 1988, Antibodies: A Laboratory Manual, Cold Spring Harbor Laboratory Press, Cold Spring Harbor, N.Y.) using an HA-specific monoclonal antibody. Briefly, the cells are labeled for 8 hours with ³⁵S-methionine (or ³⁵S-cysteine). The culture media are then collected and the cells are lysed using detergents (RIPA buffer, 150 millimolar NaCl, 1% NP-40, 0.1% SDS, 0.5% DOC, 50 millimolar Tris, pH 7.5). Both the cell lysate and the culture media are precipitated with an HA-specific monoclonal antibody. Precipitated polypeptides are then analyzed by SDS-PAGE.

Alternatively, DNA containing the 47169/33935 coding sequence is cloned directly into the polylinker of the pcDNA/Amp vector using the appropriate restriction sites. The resulting plasmid is transfected into COS cells in the manner described above, and the expression of the 47169/33935 polypeptide is detected by radiolabeling and immunoprecipitation using a 47169/33935-specific monoclonal antibody.

Equivalents

Those skilled in the art will recognize, or be able to ascertain using no more than routine experimentation, many equivalents to the specific embodiments of the invention described herein. Such equivalents are intended to be encompassed by the following claims.

1-35. (canceled)

36. A method for identifying a candidate compound capable of modulating cellular proliferation, the method comprising:
a) contacting a test compound with a sample comprising a polypeptide selected from the group consisting of:
i) a polypeptide which is at least 95% identical to the amino acid sequence of SEQ ID NO:2, wherein the polypeptide has glycosyl transferase activity;
ii) a polypeptide which is encoded by a nucleic acid molecule which is at least 95% identical to the nucleotide sequence of SEQ ID NO:1 or 3, wherein the polypeptide has glycosyl transferase activity; and
iii) a polypeptide comprising a fragment of at least 100 contiguous amino acids of SEQ ID NO:2, wherein the polypeptide has glycosyl transferase activity;
b) assaying the ability of the test compound to modulate the activity of the polypeptide;
c) contacting a cell which expresses the polypeptide with the compound identified in b); and
d) determining the effect of the compound on cellular proliferation;
thereby identifying a compound capable of modulating cellular proliferation.

37. The method of claim 36, wherein the glycosyl transferase activity is the ability to transfer an N-acetylgalactosamine moiety from uridine diphosphate to a hydroxyl moiety of a serine or threonine residue of a protein.

38. The method of claim 36, wherein the sample comprises the polypeptide, a membrane-bound form of an isolated polypeptide or a cell expressing the polypeptide.

39. The method of claim 38, wherein the cell is selected from the group consisting of a colon cell, an ovarian cell, a breast cell, a lung cell and a liver cell.

40. The method of claim 36, wherein the compound is a small molecule, a peptide, or an antibody.

41. The method of claim 36, wherein the polypeptide further comprises heterologous sequences.

42. A method for identifying a candidate compound capable of modulating cellular proliferation, the method comprising:
a) contacting a test compound with a sample comprising a polypeptide selected from the group consisting of:
i) a polypeptide comprising the amino acid sequence of SEQ ID NO:2; and
ii) a polypeptide which is encoded by the nucleotide sequence of SEQ ID NO:1 or 3;
b) assaying the ability of the test compound to modulate the activity of the polypeptide;
c) contacting a cell which expresses the polypeptide with the compound identified in b); and
d) determining the effect of the compound on cellular proliferation;
thereby identifying a compound capable of modulating cellular proliferation.

43. The method of claim 42, wherein the sample comprises the polypeptide, a membrane-bound form of an isolated polypeptide or a cell expressing the polypeptide.

44. The method of claim 43, wherein the cell is selected from the group consisting of a colon cell, an ovarian cell, a breast cell, a lung cell and a liver cell.

45. The method of claim 42, wherein the compound is a small molecule, a peptide, or an antibody.

46. The method of claim 42, wherein the polypeptide further comprises heterologous sequences.

47. A method for identifying a candidate compound capable of modulating cellular proliferation, the method comprising:
a) combining a test compound with a sample comprising a polypeptide selected from the group consisting of:
i) a polypeptide which is at least 95% identical to the amino acid sequence of SEQ ID NO:2, wherein the polypeptide has glycosyl transferase activity;
ii) a polypeptide which is encoded by a nucleic acid molecule which is at least 95% identical to the nucleotide sequence of SEQ ID NO:1 or 3, wherein the polypeptide has glycosyl transferase activity; and
iii) a polypeptide comprising a fragment of at least 100 contiguous amino acids of SEQ ID NO:2, wherein the polypeptide has glycosyl transferase activity;
under conditions suitable for binding;
b) selecting a compound capable of binding to the polypeptide;
c) contacting a cell which expresses the polypeptide with the compound identified in b); and
d) determining the effect of the compound on cellular proliferation;
thereby identifying a compound capable of modulating cellular proliferation.

48. The method of claim 47, wherein the glycosyl transferase activity is the ability to transfer an N-acetylgalactosamine moiety from uridine diphosphate to a hydroxyl moiety of a serine or threonine residue of a protein.

49. The method of claim 47, wherein the sample comprises the polypeptide, a membrane-bound form of an isolated polypeptide or a cell expressing the polypeptide.

50. The method of claim 49, wherein the cell is selected from the group consisting of a colon cell, an ovarian cell, a breast cell, a lung cell and a liver cell.

51. The method of claim 47, wherein the compound is a small molecule, a peptide, or an antibody.

52. The method of claim 47, wherein the polypeptide further comprises heterologous sequences.

53. The method of claim 47, wherein the binding of the test compound to the polypeptide is determined by a method selected from the group consisting of:
a) direct detecting of test compound/polypeptide binding;
b) a competition binding assay;
c) an immunoassay;
d) a yeast two-hybrid assay; and
e) an assay detecting the formation of a covalent bond between a carbohydrate (i.e., saccharide) moiety and a hydroxyl or amino moiety of a protein.

54. A method for identifying a candidate compound capable of modulating cellular proliferation, the method comprising:
a) combining a test compound with a sample comprising a polypeptide selected from the group consisting of:
i) a polypeptide comprising the amino acid sequence of SEQ ID NO:2; and
ii) a polypeptide which is encoded by the nucleotide sequence of SEQ ID NO:1 or 3;
under conditions suitable for binding;
b) selecting a compound capable of binding to the polypeptide;
c) contacting a cell which expresses the polypeptide with the compound identified in b); and
d) determining the effect of the compound on cellular proliferation;
thereby identifying a compound capable of modulating cellular proliferation.

55. The method of claim 54, wherein the sample comprises the polypeptide, a membrane-bound form of an isolated polypeptide or a cell expressing the polypeptide.

56. The method of claim 55, wherein the cell is selected from the group consisting of a colon cell, an ovarian cell, a breast cell, a lung cell and a liver cell.

57. The method of claim 54, wherein the compound is a small molecule, a peptide, or an antibody.

58. The method of claim 54, wherein the polypeptide further comprises heterologous sequences.

59. The method of claim 54, wherein the binding of the test compound to the polypeptide is determined by a method selected from the group consisting of:
a) direct detecting of test compound/polypeptide binding;
b) a competition binding assay;
c) an immunoassay;
d) a yeast two-hybrid assay; and
e) an assay detecting the formation of a covalent bond between a carbohydrate (i.e., saccharide) moiety and a hydroxyl or amino moiety of a protein.
